(19) United States
(12) Patent Application Publication
(10) Pub. No.: US 2025/0114149 A1
(43) Pub. Date: Apr. 10, 2025

(54) IMAGE PROCESSING SYSTEM, IMAGE PROCESSING METHOD, AND INFORMATION STORAGE MEDIUM

(71) Applicant: OLYMPUS CORPORATION, Tokyo (JP)

(72) Inventors: Shoei TSURUTA, Tokyo (JP); Kantaro NISHIKAWA, Tokyo (JP); Hisatsugu TAJIMA, Tokyo (JP); Takeshi ARAI, Tokyo (JP); Tetsuri KUWAZURU, Tokyo (JP)

(73) Assignee: OLYMPUS CORPORATION, Tokyo (JP)

(21) Appl. No.: 18/910,360

(22) Filed: Oct. 9, 2024

Related U.S. Application Data
(60) Provisional application No. 63/543,371, filed on Oct. 10, 2023.

(51) Int. Cl. A61B 34/20; A61B 90/00; G06T 5/50; G06T 7/11; G06T 7/13; G06T 7/246

(52) U.S. Cl. CPC A61B 34/20; A61B 90/37; G06T 5/50; G06T 7/11; G06T 7/13; G06T 7/246; A61B 2034/2055; G06T 2207/10068; G06T 2207/20221

(57) ABSTRACT

An image processing system includes one or more processors comprising hardware configured to sequentially acquire time-series images captured by an endoscope, a dispose an evaluation mesh including a plurality of analysis points in a freely-selected timing image out of the time-series images, deform the evaluation mesh in each image of the time-series images so that each analysis point in each image of the time-series images tracks a characteristic point of an object located on each analysis point in the freely-selected timing image in which the evaluation mesh is disposed, calculate a deformation quantity of each cell of the evaluation mesh based on magnitude and a direction of a movement quantity of each analysis point in each image, and present information regarding deformation of the evaluation mesh based on the calculated deformation quantity.

### CROSS REFERENCE TO RELATED APPLICATIONS

This application is based upon and claims the benefit of priority to U.S. Provisional Patent Application No. 63/543,371 filed on Oct. 10, 2023, the entire contents of which are incorporated herein by reference.

### BACKGROUND

The specification of U.S. Unexamined Patent Application Publication No. 2013/0041368 discloses a remote control method used in surgery. In this method, a sensor detects force applied to a tissue, and a system uses the force detected by the sensor to return haptic feedback to a user's operation step. Additionally, the specification of U.S. Unexamined Patent Application Publication No. 2021/0322121 discloses a visual haptic system for a robotic surgical platform. This system uses a visual haptic model to refer to an image and classify the image into a set of force levels. The visual haptic model has been subjected to machine learning to classify the image into the set of force levels using a force level of force applied to a tissue and a video that shows the tissue. Furthermore, the specification of U.S. Unexamined Patent Application Publication No. 2021/0322121 discloses that the system performs mapping of a visual appearance in an image into force levels, the force level includes a tightness level of a surgical knot, the force level includes a tension level, and the system generates a skill score.

### SUMMARY

In accordance with one of some aspect, there is provided an image processing system comprising:
one or more processors comprising hardware configured to:
sequentially acquire time-series images captured by an endoscope dispose an evaluation mesh including a plurality of analysis points in a freely-selected timing image out of the time-series images,
   deform the evaluation mesh in each image of the time-series images so that each analysis point in each image of the time-series images tracks a characteristic point of an object located on each analysis point in the freely-selected timing image in which the evaluation mesh is disposed,
   calculate a deformation quantity of each cell of the evaluation mesh based on magnitude and a direction of a movement quantity of each analysis point in each image, and
   present information regarding deformation of the evaluation mesh based on the calculated deformation quantity.

In accordance with one of some aspect, there is provided an image processing method comprising:
sequentially acquiring time-series images captured by an endoscope;
disposing an evaluation mesh including a plurality of analysis points in a freely-selected timing image out of the time-series images;
deforming the evaluation mesh in each image of the time-series images so that each analysis point in each image of the time-series images tracks a characteristic point of an object located on each analysis point in the freely-selected timing image in which the evaluation mesh is disposed;
calculating a deformation quantity of each cell of the evaluation mesh based on magnitude and a direction of a movement quantity of each analysis point in each image; and
presenting information regarding deformation of the evaluation mesh based on the calculated deformation quantity.

In accordance with one of some aspect, there is provided a non-transitory information storage medium storing a program that causes a computer to execute:
sequentially acquiring time-series images captured by an endoscope;
disposing an evaluation mesh including a plurality of analysis points in a freely-selected timing image out of the time-series images;
deforming the evaluation mesh in each image of the time-series images so that each analysis point in each image of the time-series images tracks a characteristic point of an object located on each analysis point in the freely-selected timing image in which the evaluation mesh is disposed;
calculating a deformation quantity of each cell of the evaluation mesh based on magnitude and a direction of a movement quantity of each analysis point in each image; and
presenting information regarding deformation of the evaluation mesh based on the calculated deformation quantity.

### BRIEF DESCRIPTION OF THE DRAWINGS

FIG. 1 illustrates an example of an endoscopic surgery scene to which a method in accordance with the present embodiment is applicable.

FIG. 2 illustrates a basic method of presenting a pulling state of a tissue in the present embodiment.

FIG. 3 illustrates a basic method of presenting a pulling state of a tissue in the present embodiment.

FIG. 4 illustrates a basic method of presenting a pulling state of a tissue in the present embodiment.

FIG. 5 illustrates a configuration example of a medical system.

FIG. 6 illustrates a configuration example of an image processing system.

FIG. 7 illustrates a flow of processing executed by the image processing system.

FIG. 8 is a table describing a determination target and a determination method in a pulling scene determining step.

FIG. 9 illustrates a signal action.

FIG. 10 is a view for describing analysis of deformation when the start of movement of a tissue is recognized.

FIG. 11 is a table describing a pulling estimation range and a determination method in a pulling range estimating step.

FIG. 12 is a view for describing a method of estimating a region in which tension is applied to a tissue.

FIG. 13 is a view for describing a method of detecting a region in which excessive tension is applied.

FIG. 14 is a view for describing a method of estimating a pulling range in a case where a non-uniform tissue exists in a mixed manner.

FIG. 15 illustrates a relationship between stress and a deformation quantity of each of a hardly stretchable tissue and an easily stretchable tissue.

FIG. 16 is a view for describing another method of estimating the pulling range.

FIG. 17 is a view for describing the above-mentioned other method in detail.

FIG. 18 is a view for describing the above-mentioned other method in detail.

FIG. 19 is a view for describing the above-mentioned other method in detail.

FIG. 20 is a view for describing the above-mentioned other method in detail.

FIG. 21 is a table describing processing for presentation and a presentation method in a presentation information processing step.

FIG. 22 illustrates a specific example of the presentation method.

FIG. 23 illustrates a specific example of the presentation method.

FIG. 24 illustrates a specific example of the presentation method.

FIG. 25 illustrates a specific example of the presentation method.

FIG. 26 illustrates a specific example of the presentation method.

FIG. 27 illustrates a specific example of the presentation method.

FIG. 28 illustrates a specific example of the presentation method.

FIG. 29 illustrates a specific example of the presentation method.

FIG. 30 illustrates a specific example of the presentation method.

FIG. 31 illustrates a specific example of the presentation method.

FIG. 32 illustrates a specific example of the presentation method.

FIG. 33 illustrates a specific example of the presentation method.

FIG. 34 illustrates a specific example of the presentation method.

FIG. 35 illustrates a specific example of the presentation method.

FIG. 36 is a view for describing a state where an evaluation region straddles a pulled tissue and a background.

FIG. 37 is a view for describing a method in accordance with a second embodiment.

FIG. 38 illustrates a configuration example of a medical system in the second embodiment.

FIG. 39 illustrates a flow of processing in an image processing system in the second embodiment.

FIG. 40 illustrates a processing example of the image processing system in the second embodiment.

FIG. 41 illustrates an example of exclusion of a range in which there is no movement.

FIG. 42 illustrates an example of narrowing using segmentation.

FIG. 43 illustrates an example of narrowing using detection of a contour.

FIG. 44 illustrates an example of narrowing using depth information.

FIG. 45 illustrates an example of narrowing using detection in a pulling direction.

FIG. 46 illustrates a first detailed configuration example of an evaluation region setting section.

FIG. 47 illustrates a flow of processing executed by the evaluation region setting section in the first detailed configuration example.

FIG. 48 illustrates an example of a threshold for a movement quantity.

FIG. 49 illustrates a second detailed configuration example of an evaluation region setting section.

FIG. 50 illustrates a flow of processing executed by the evaluation region setting section in the second detailed configuration example.

FIG. 51 illustrates a third detailed configuration example of an evaluation region setting section.

FIG. 52 illustrates a fourth detailed configuration example of an evaluation region setting section.

FIG. 53 illustrates an example of a method of selecting the evaluation region.

FIG. 54 illustrates a fifth detailed configuration example of an evaluation region setting section.

FIG. 55 illustrates a sixth detailed configuration example of an evaluation region setting section.

FIG. 56 illustrates a flow of processing executed by the evaluation region setting section in the sixth detailed configuration example.

FIG. 57 illustrates a seventh detailed configuration example of an evaluation region setting section.

FIG. 58 illustrates a flow of processing executed by the evaluation region setting section in the seventh detailed configuration example.

FIG. 59 is a view for describing that the evaluation region is gradually narrowed down over time.

FIG. 60 illustrates an eighth detailed configuration example of an evaluation region setting section.

FIG. 61 illustrates a flow of processing executed by the evaluation region setting section in the eighth detailed configuration example.

FIG. 62 illustrates a ninth detailed configuration example of an evaluation region setting section.

FIG. 63 illustrates a flow of processing executed by the evaluation region setting section in the ninth detailed configuration example.

FIG. 64 illustrates an example of a change in threshold.

FIG. 65 illustrates an example of extension of the evaluation region.

FIG. 66 illustrates a tenth detailed configuration example of an evaluation region setting section.

FIG. 67 illustrates a flow of processing executed by the evaluation region setting section in the tenth detailed configuration example.

FIG. 68 illustrates an example of options for the evaluation region.

FIG. 69 illustrates an example of options for the evaluation region.

FIG. 70 illustrates an example of options for the evaluation region.

FIG. 71 illustrates an example of setting the evaluation region near a distal end of a treatment tool.

FIG. 72 illustrates a detailed flow in an image processing step in a third embodiment.

FIG. 73 illustrates a detailed flow in a region analyzing step.

FIG. 74 is a view for describing a non-attention tissue recognizing step.

FIG. 75 is a view for describing a flow vector calculating step.

FIG. 76 is a view for describing an evaluation mesh analyzing step.

FIG. 77 is a view for describing an evaluation mesh updating step.

FIG. 78 is a view for describing a modification of the evaluation mesh analyzing step.

FIG. 79 is a view for describing a modification of the evaluation mesh analyzing step.

FIG. 80 is a view for describing a modification of the evaluation mesh updating step.

FIG. 81 is a view for describing a basic concept.

FIG. 82 illustrates an example of a preliminarily registered pattern.

FIG. 83 illustrates a flow of processing in an image processing system in a fourth embodiment.

FIG. 84 illustrates an example regarding training data and a recognition result in detection of a device.

FIG. 85 illustrates an example regarding training data and a recognition result in detection of contact.

FIG. 86 is a view for describing a tissue deformation recognizing step.

FIG. 87 illustrates a display example of a main monitor and a display example of a sub monitor.

FIG. 88 illustrates an example of presentation information indicating loosening of a tissue.

FIG. 89 illustrates a first flow of presentation of loosening.

FIG. 90 illustrates a second flow of presentation of loosening.

FIG. 91 illustrates an example of presentation information in assistance for an operator's pulling.

FIG. 92 illustrates a first flow of assistance for the operator's pulling.

FIG. 93 illustrates an example of a relationship between a deformation quantity and brightness or density of a color.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. In the following disclosure, a “ . . . section“and a” . . . step” can be replaced with each other. For example, in a case where it is described that a processor executes the “ . . . step”, the processor may include the “ . . . section“as hardware or software for executing the” . . . step”, and vice versa.

### 1. Method

In a treatment using an endoscope, it may be preferable that a treatment be performed in a state where tension appropriate for a treatment target is applied. At this time, there is an issue regarding how to detect whether or not appropriate tension is applied to the treatment target. The above-mentioned two documents each disclose the method of detecting force itself. However, the method disclosed in the specification of U.S. Unexamined Patent Application Publication No. 2013/0041368 is on the premise that the sensor detects force applied to a tissue, and it is impossible to detect force without using the sensor nor return haptic feedback. Additionally, the specification of U.S. Unexamined Patent Application Publication No. 2021/0322121 requires enormous training cost to perform machine learning to classify force levels from images. Additionally, the specification of U.S. Unexamined Patent Application Publication No. 2021/0322121 does not disclose a specific presentation method that is useful for an operator. Furthermore, in the specification of U.S. Unexamined Patent Application Publication No. 2021/0322121, it is necessary to use information regarding force that cannot be acquired only from an image in a training phase.

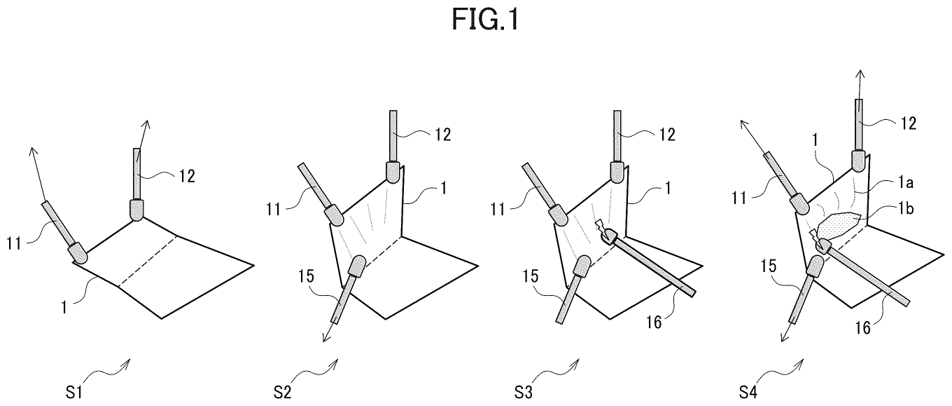

FIG. 1 illustrates a scene in which an operator and an assistant perform an energy treatment while cooperatively pulling a tissue as an example of an endoscopic surgery scene to which a method in accordance with the present embodiment is applicable. As illustrated in step S1, the assistant uses forceps 11 and 12 to grip and pull a tissue 1, and thereby develops a treatment region. As illustrated in step S2, after the assistant develops the treatment region of the tissue 1, the operator uses forceps 15 to grip and pull the tissue. That is, the operator separates the treatment target of the tissue 1 from its surroundings and pulls the tissue 1 to ensure safety. As illustrated in step S3, after the success in the pulling of the treatment target of the tissue 1, the operator uses an energy treatment tool 16 to perform a treatment on the tissue 1 in a pulling range. As illustrated in step S4, examples of the treatment include incision, and a new surface 1b of the tissue appears due to incision, which generates a loose surface 1a on the tissue 1.

In a case where loosening occurs in the tissue 1 due to the treatment in this manner, the assistant or the operator pulls the tissue 1 again. In the method in accordance with the present embodiment, which will be described later, information is presented to a user so as to allow the user to determine a pulling state of the tissue 1 in at least one of steps S1 to S4. The user is, for example, a surgeon, and the surgeon includes the operator and the assistant. The information may be presented to both the operator and the assistant, or may be presented to either the operator or the assistant.

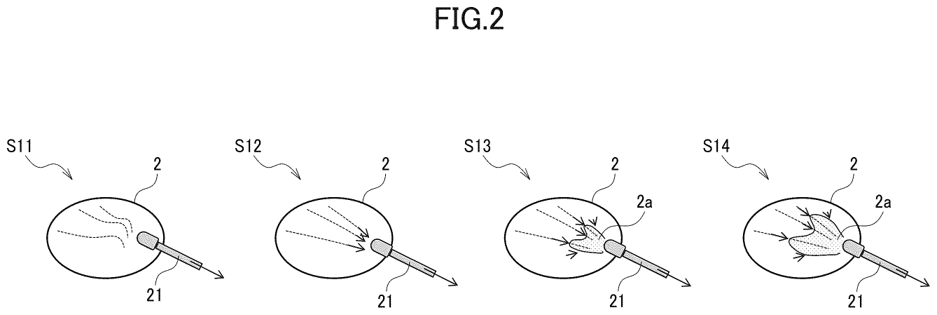

A basic method of presenting a pulling state of a tissue in the present embodiment will be described with reference to FIGS. 2 to 4.

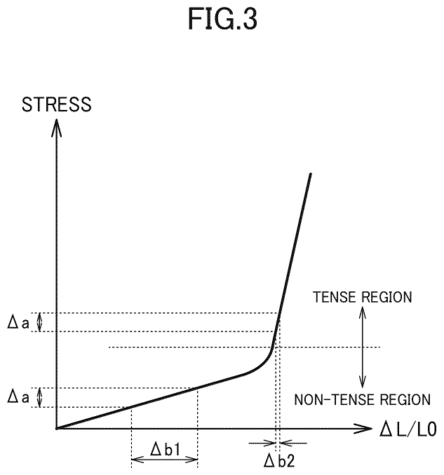

FIG. 2 illustrates a behavior of the tissue when the tissue is pulled. Forceps 21 that pull the tissue may be operated by either the operator or the assistant. As illustrated in step S11, the tissue 2 has slack due to residual stress in the tissue 2, a self-weight of the tissue 2, or the like before being pulled. When the tissue 2 is pulled from this state by the forceps 21, the slack in the tissue 2 is removed for a while. As illustrated in step S12, when the tissue 2 is further pulled by the forceps 21 after the removal of the slack in the tissue 2, the tissue 2 starts to stretch or become strained. As illustrated in step S13, when the tissue 2 is further pulled by the forceps 21, the stretch relative to the pulling stops, and a tense region 2a in which tension is generated appears. As illustrated in FIG. 3, the tense region 2a is a region in which stress in the tissue 2 rises and tension appropriate for the treatment is considered to be applied to the tissue 2. The tissue 2 in the surroundings of the tense region 2a stretches relative to the pulling, and the tissue 2 on the outer periphery thereof makes translational movement relative to the pulling. As illustrated in step S14, when the tissue 2 keeps further pulled by the forceps 21, a region in the surroundings of the tense region 2a in step S13 is added to the tense region 2a, and the tissue 2 in the surroundings thereof becomes a region stretchable relative to the pulling.

In this manner, there is a relationship among the pulling of the tissue 2, the deformation of the tissue 2, and tension applied to the tissue 2. In the present embodiment, with use of this relationship, it is possible to present pulling information to a user without directly detecting a force applied to the tissue 2. That is, detecting the deformation of the tissue 2 from an image and presenting information regarding the deformation to the user allows the user to determine the pulling state of the tissue 2, tension applied to the tissue 2, or the like by seeing the presented information. For example, the operator confirms that a treatment target region is included in the tense region 2a from the presentation information regarding the deformation, and can thereby perform a treatment on the treatment target region with an energy treatment tool.

FIG. 3 illustrates a relationship between a deformation quantity of the tissue and stress. While the deformation quantity may have various forms as described later, an example of using ΔL/L0 as the deformation quantity is described here. ΔL/L0 is a ratio of stretch ΔL of the tissue to a pulling distance L0. A change Δb2 in ΔL/L0 with respect to a stress change Δa in the tense region is smaller than a change Δb1 in ΔL/L0 with respect to the stress change Δa in a non-tense region. That is, the deformation quantity of the tissue relative to the pulling is small in the tense region and large in the non-tense region. This is as described with reference to FIG. 2, and indicates that there is a relationship among the pulling of the tissue 2, the deformation of the tissue 2, and tension applied to the tissue 2.

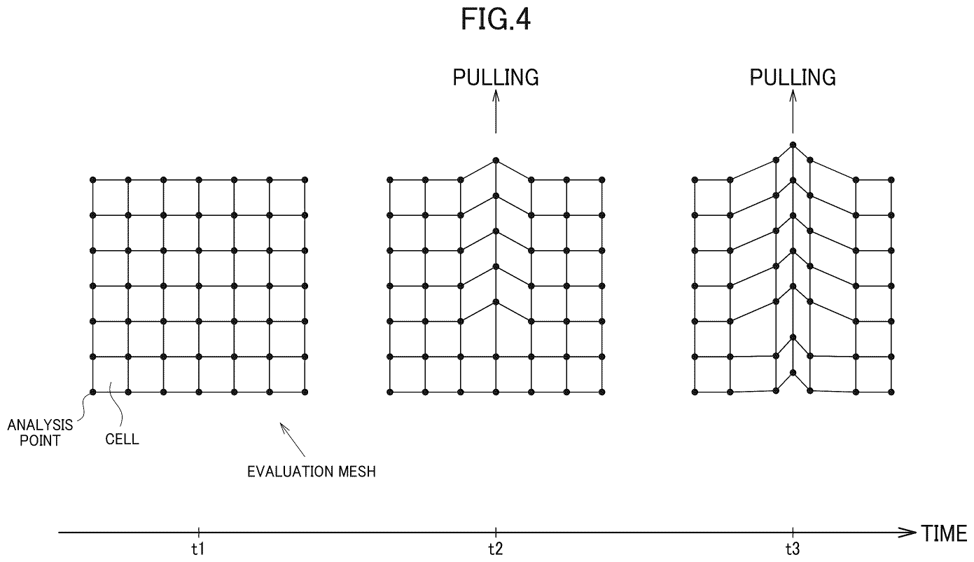

FIG. 4 illustrates a method of detecting the deformation of the tissue in the present embodiment. Assume that times t1 to t3 are freely-selected times, and t2 is a time after t1, and t3 is a time after t2. For example, the times t1 to t3 are times at certain intervals such as frames of a movie. An image processing system in accordance with the present embodiment sets an evaluation mesh in an endoscope image at the time t1. The time t1 may be a freely-selected timing. The evaluation mesh is also referred to as a lattice or a grid. An analysis target point discretely disposed in the evaluation mesh, that is, each intersection point between a transverse line and a vertical line in the evaluation mesh is referred to as an analysis point. The analysis point is referred to as a lattice point or a grid point. A region having the nearest four analysis points as apexes in the evaluation mesh, that is, a region surrounded by transverse lines and vertical lines in the evaluation mesh is referred to as a cell. Note that directions of the lines that define the evaluation mesh are not limited to traverse and vertical directions. The image processing system tracks deformation of an object on the image and moves the analysis points at the times t2 and t3 as if the analysis points stuck to the object. The tracking is implemented by movement detection using an image characteristic quantity, for example, Optical Flow detection or the like.

The deformation quantity of the evaluation mesh represents a deformation quantity of the tissue due to pulling. In the present embodiment, the deformation quantity of the evaluation mesh is detected from an image without use of a sensor that detects force, and display depending on the deformation quantity is performed. As described above, the deformation quantity of the tissue is related to a pulling state or tension, and the user can determine the pulling state of the tissue or tension by seeing the display depending on the deformation quantity.

The deformation quantity of the evaluation mesh is a quantity defined by each analysis point or each cell, is not limited to a scalar quantity, may be a quantity represented by a vector or a tensor, and may be, for example, displacement, a movement quantity, stretch, strain, or the like.

An example of the deformation quantity is the displacement of each analysis point. The displacement mentioned herein may be only the magnitude of the displacement, or may be a vector including the magnitude and direction of the displacement. The displacement may be displacement using the position of the analysis point at a time point as a criterion, or may be displacement at predetermined intervals such as frames. Alternatively, the displacement may be displacement relative to the position of an analysis point in the surroundings. Note that, since the displacement of a point is synonymous with the movement of the point, assume that the displacement of the point and the movement of the point are used without being distinguished from each other, and the displacement and the movement can be replaced with each other.

Another example of the deformation quantity is the stretch or contraction of the cell. The stretch or the contraction mentioned herein is a change in length of a side of the cell, or a change in distance between two facing sides of the cell. Alternatively, the stretch or the contraction may be represented by a strain component such as main strain. The stretch or the contraction may be stretch or contraction when the shape of the cell at a time point serves as a criterion, or stretch or contraction at predetermined intervals such as frames.

Still another example of the deformation quantity is the strain of the cell. The “strain” mentioned herein may be a tensor quantity represented by a plurality of components, may be part of a plurality of components included in the tensor quantity, or may be strain in a specific direction such as main strain and sub strain. Alternatively, the deformation quantity may be a change quality of the strain of the cell. The change quantity of the strain may be a change quantity when a strain in the evaluation mesh at a time point serves as a criterion, or may be a change quantity at predetermined intervals such as frames.

The deformation quantity may not be the above-mentioned quantity itself, but may be a quantity that is calculated using the above-mentioned quantity of various kinds. For example, deformation quantities of some analysis points or cells in the surroundings may be averaged, or temporal or spatial filtering may be performed on the deformation quantity of the analysis point or the cell. Note that a “deformation quantity” and specific examples of “displacement”, “movement”, “stretch”, “strain”, and the like may be replaced with each other in the following description.

First to fifth embodiments using the above-mentioned method will be described below. Contents of the first to fifth embodiments can be implemented in combination as appropriate. For example, contents of one of the second to fifth embodiments or contents of a plurality of the second to fifth embodiments may be combined with contents of the first embodiment. Even in a case where a description about a configuration, processing, or the like is omitted in an embodiment, contents of a configuration, processing, or the like described in another embodiment can be applied.

### 2. First Embodiment

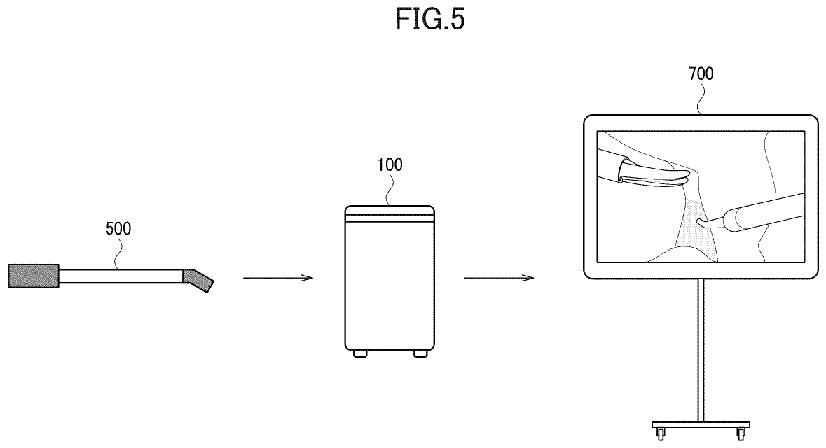

FIG. 5 illustrates a configuration example of a medical system. The medical system includes an endoscope 500, an image processing system 100, and a monitor 700.

The endoscope 500 is inserted into the inside of the body of a patient, captures an in-vivo image, and transmits image data thereof to the image processing system 100. The endoscope 500 captures images in a time-series manner, and the images are referred to as time-series images. Additionally, each image included in the time-series images is referred to as an endoscope image. The time-series images are, for example, endoscope images in each frame of a movie captured by the endoscope 500, or endoscope images extracted at predetermined intervals from the movie. The endoscope 500 may be a rigid scope such as a laparoscope or an arthroscope or a flexible scope such an intestinal endoscope.

The image processing system 100 detects a deformation quantity of a tissue from endoscope images, and performs display depending on the deformation quantity so as to be superimposed on the endoscope images on the monitor 700. The image processing system 100 may perform image processing on endoscope images captured by the endoscope 500 in real time. Alternatively, endoscope images are recorded in a storage such as a hard disk drive and a non-volatile memory, and the image processing system 100 may perform image processing on the endoscope images recorded in the storage. Note that the endoscope system normally includes the endoscope 500 and an endoscope control device, but the image processing system 100 may be built into the endoscope control device. Alternatively, the image processing system 100 may be a system provided separately from the endoscope control device. In this case, the endoscope control device may generate endoscope images from image signals from the endoscope 500, and output the endoscope images to the image processing system 100.

Note that the medical system may include a plurality of monitors, and the image processing system 100 may display different information on respective monitors. For example, the medical system may include a first monitor and a second monitor, the first monitor may display original endoscope images, and the second monitor may display endoscope images on which information depending on a result of analyzing the evaluation mesh is superimposed. Alternatively, so-called picture-in-picture may be employed. In the picture-in-picture, information depending on an analysis result is displayed so as not to be superimposed on the original endoscope images on the first monitor. Alternatively, both the first monitor and the second monitor may display endoscope images on which information depending on a result of analyzing the evaluation mesh is superimposed. At this time, the first monitor may display endoscope images on which information for the assistant is superimposed, and the second monitor may display endoscope images on which information for the operator is superimposed.

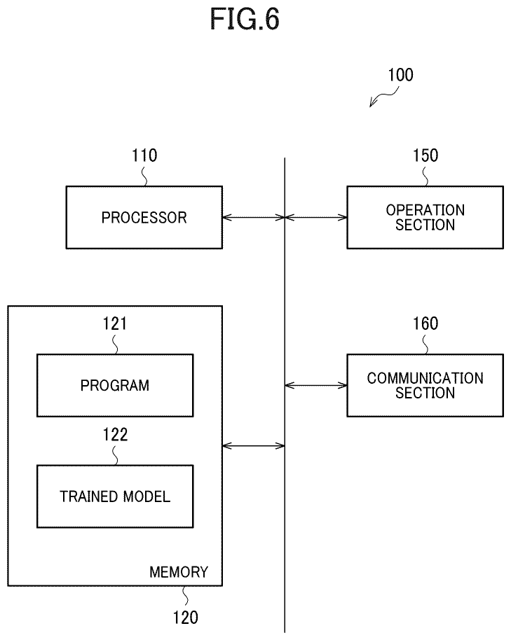

FIG. 6 illustrates a configuration example of the image processing system 100. The image processing system 100 includes a processor 110, a memory 120, and a communication section 160. Additionally, the image processing system 100 may further include an operation section 150. The image processing system 100 may be, for example, an information processing device such as a personal computer (PC) and a server, or may be a cloud system to which a plurality of information processing devices is connected via a network.

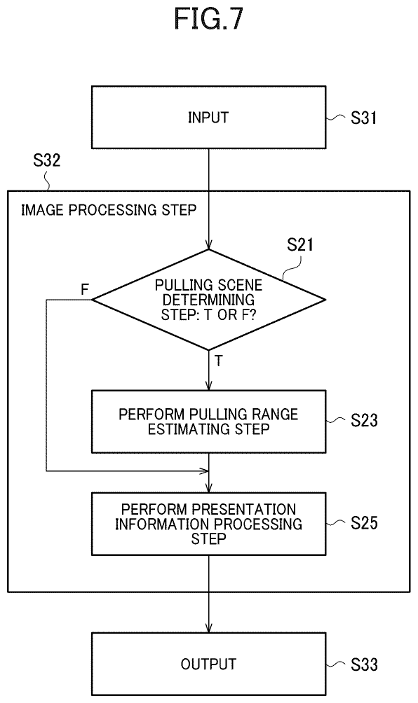

The memory 120 stores a program 121 in which various kinds of processing contents of processing executed by the image processing system 100 are described. The processor 110 reads the program 121 from the memory 120 and executes the program 121 to execute various kinds of processing. For example, the processor 110 executes processing of each step, which will be described later with reference to FIG. 7. In a case where machine learning is used in processing, the memory 120 may store a trained model 122 obtained by machine learning. The processor 110 uses the trained model 122 to execute the processing using machine learning. The trained model 122 does not necessarily mean only a model that has been especially trained to execute the present embodiment, and may be an existing model that has been trained for a general purpose. The trained model 122 may include a plurality of machine learning models. The processor 110 and the memory 120 may have, for example, the following configurations.

The processor 110 includes hardware. The processor 110 is, for example, a central processing unit (CPU), a graphics processing unit (GPU), a microcomputer, a digital signal processor (DSP), or the like. Alternatively, the processor 110 may be an application specific integrated circuit (ASIC), a field programmable gate array (FPGA), or the like. The processor 110 may be configured to include one or more of the CPU, the GPU, the microcomputer, the DSP, the ASIC, the FPGA, and the like. The memory 120 is, for example, a semiconductor memory, which is a volatile memory or a non-volatile memory. Alternatively, the memory 120 may be a magnetic storage device such as a hard disk device, or may be an optical storage device such as an optical disk device. The trained model 122 stored in the memory 120 may include, for example, a program in which algorithms of artificial intelligence (AI) are described, data used in the program, and the like. For example, the trained model 122 may include a neural network such as a convolutional neural network (CNN). In this case, the trained model 122 includes a program in which algorithms of the neural network are described, a weight parameter and a bias applied between nodes of the neural network, and the like. The neural network includes an input layer that takes data, an intermediate layer that executes calculation processing based on data input via the input layer, and an output layer that outputs a recognition result based on a calculation result output from the intermediate layer. The program 121, the trained model 122, or both the program 121 and the trained model 122 may be stored in a non-transitory information storage medium, which is a computer readable storage medium. The information storage medium is, for example, an optical disk, a memory card, a hard disk drive, a semiconductor memory, or the like. The semiconductor memory is, for example, a read-only memory (ROM) or a non-volatile memory. The processor 110 loads the program 121 stored in the information storage medium in the memory 120, and performs various kinds of processing based on the program 121.

The communication section 160 performs communication with the outside of the image processing system 100. The communication section 160 may include, for example, a connector or an interface that connects the endoscope 500, a connector or an interface that connects the monitor 700, or an interface for network connection such as a local area network (LAN).

The operation section 150 accepts operation input to the image processing system 100 from the user. The operation section 150 is, for example, a button, a switch, a dial, a lever, a keyboard, or a pointing device. Alternatively, the operation section 150 may be implemented by a touch panel provided on the monitor 700.

The processor 110 may perform processing using machine learning and processing based on a rule in a mixed manner. Examples will be described below. Note that processing described as an example of the processing using machine learning may be implemented by the processing based on the rule, or processing described as an example of the processing based on the rule may be implemented by the processing using machine learning.

Examples of processing using machine learning
Detection of a position of a treatment tool in an image.
Detection of a region of the treatment tool in the image. These are for ignoring the treatment tool that interferes with tracking of an analysis point.
Recognition of a type of the treatment tool in the image. This is for distinguishing whether a person who operates the treatment tool is the operator or the assistant, whether a hand that operates the treatment tool is a right hand or a left hand, and whether the treatment tool is forceps or an energy treatment tool.
Detection of a contact state or a gripping state between the treatment tool and a biotissue. This is for using a change in the contact state or the gripping state as a trigger. The trigger is a trigger for start of analysis, end of analysis, start of presentation, or end of presentation.
Estimation of an evaluation region. This is for estimating an analysis range. The evaluation region is a region in which the evaluation mesh is set.
Recognition of a type of a tissue in a pulling range. This is for determining in advance whether a tissue is a tissue with different elasticity.

Example of the processing based on the rule
Tracking of a characteristic point. This is for analyzing deformation over time using a method such as Optical Flow. Information obtained by Optical Flow or the like is analyzed by the processing based on the rule and a characteristic point is tracked. Regarding Optical Flow itself, a classical method may be used, or Recurrent All-Pairs Field Transforms (RAFT) using AI or the like may be used.
Various kinds of correction processing. The correction processing is, for example, correction of a change in camera angle, and correction of translation movement of a camera, scaling of the camera, rotation of the camera, or shake of the camera, or the like. Alternatively, the correction processing is correction of drift, which is unintentional shift of an analysis result due to noise.

FIG. 7 illustrates a flow of processing executed by the image processing system 100. In step S31, endoscope images are input to the processor 110. The processor 110 executes an image processing step in step S32, and outputs a result of the image processing step to the monitor 700 in step S33.

An image processing step S32 includes a pulling scene determining step S21, a pulling range estimating step S23, and a presentation information processing step S25. In step S21, the processor 110 determines whether a scene is a scene in which a target pulling range should be estimated from the endoscope images or the like. That is, the processor 110 determines a timing of starting estimation of a pulling state and a timing of ending the estimation of the pulling state. In a case of determining that the scene is the scene in which the pulling range should be estimated (T: True), the processor 110 estimates the pulling range in step S23. In a case of determining that the scene is not the scene in which the pulling range should be estimated (F: False), the processor 110 does not estimate the pulling range in step S23, or transmits, to step S25, information indicating that presentation information is not to be added. In step S23, the processor 110 calculates a movement quantity of a tissue in the images, causes analysis points to track the movement of the tissue, and estimates the pulling range from information regarding the evaluation mesh. In step S25, the processor 110 modifies a result of estimation of the pulling range as information necessary for the user, and performs processing on the presentation information to enable monitor presentation useful for the user. Note that the processor 110 may execute a region setting step of setting a region for estimating the pulling state between step S21 and step S23.

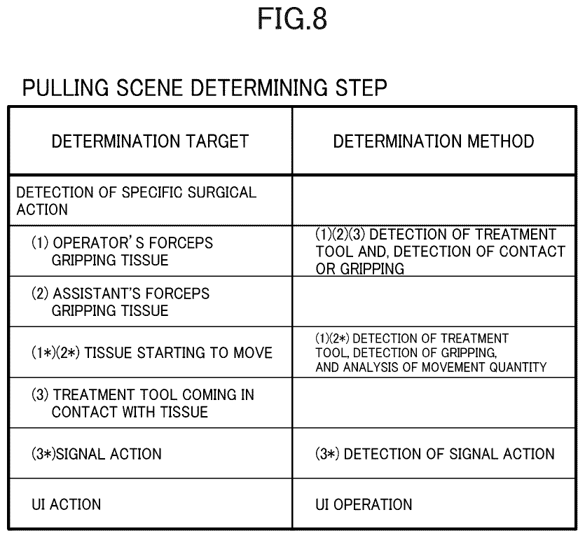

Details of the pulling scene determining step S21 are now described. FIG. 8 is a table describing a determination target and a determination method in the pulling scene determining step S21. A “UI” in the table is an abbreviation for a user interface.

The output in this determining step may be a label on information processing indicating the pulling scene or may be a specific signal such as an electric pulse. Additionally, in a case where the scene is not the pulling scene, the processor 110 may skip the processing in the pulling range estimating step without adding the label on the information processing, or may execute the processing in the pulling range estimating step in a state where the label on the information processing indicating that it is not the pulling scene is added.

Each item of the table in FIG. 8 is now described. A bracketed number such as (1) represents an embodiment. An item to which an identical number is added represents a corresponding embodiment. For example, (1) in “DETERMINATION TARGET” and (1) in “DETERMINATION METHOD” represent a corresponding embodiment. * means a modification. For example, (1*) is a modification of (1).

### [Determination Target]

(1) The processor 110 recognizes the operator's forceps gripping the tissue as the pulling scene.

(2) The processor 110 recognizes at least one pair of forceps held by the assistant gripping the tissue as the pulling scene.

(1*) (2*) The processor 110 may recognize the tissue starting to move as the pulling scene.

(3) The processor 110 recognizes the energy treatment tool coming in contact with the tissue as the pulling scene.

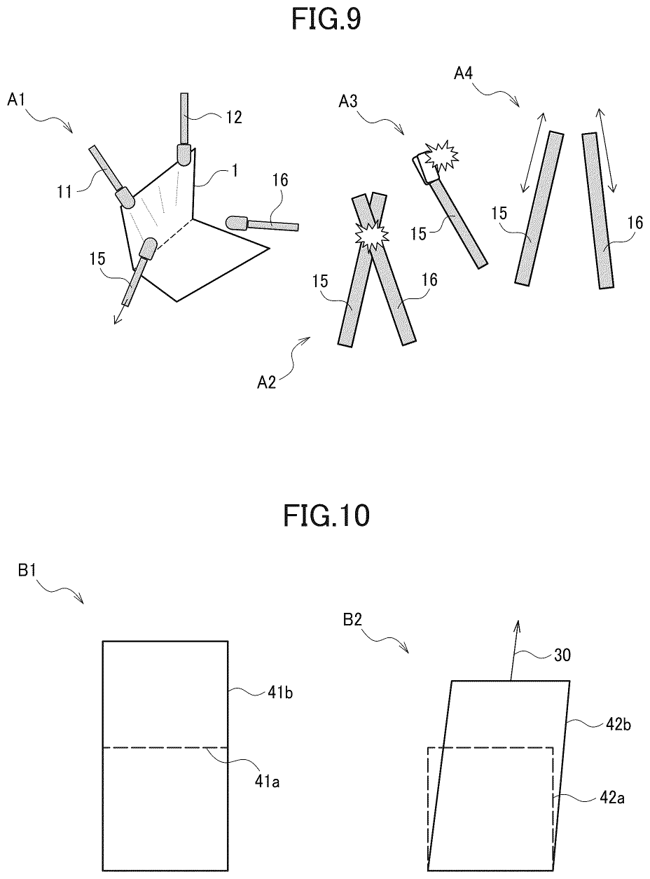

(3*) The processor 110 detects a signal action from the endoscope images to determine the pulling scene. FIG. 9 illustrates examples of signal actions in A1 to A4. A1 illustrates a state where the operator grips the tissue 1 with the forceps 15 and the energy treatment tool 16 is within a field of view since the assistant has started development as a signal action. A2 illustrates a signal action performed by the operator to cross and contact two treatment tools such as the forceps 15 and the energy treatment tool 16. A3 illustrates a signal action performed by the operator to grip a space, which is not the tissue, twice with the forceps 15. A4 illustrates a signal action performed by the operator to reciprocate shafts of two treatment tools such as the forceps 15 and the energy treatment tool 16 twice.

### [Determination Method]

(1)(2) The processor 110 uses processing of detecting the treatment tool from the endoscope images and processing of detecting contact between the treatment tool and the tissue or gripping of the tissue with the treatment tool from the endoscope images for recognition in (1) and (2) in “DETERMINATION TARGET”.

(3) The processor 110 uses the processing of detecting the treatment tool from the endoscope images and the processing of detecting contact between the treatment tool and the tissue or gripping of the tissue with the treatment tool from the endoscope images for recognition in (3) in “DETERMINATION TARGET”. Specifically, the processor 110 detects the energy treatment tool from the endoscope images, and detects contact of the energy treatment tool with the tissue from the endoscope images.

(3*) The processor 110 uses processing of detecting the signal action from the endoscope images for recognition in (3*) in “DETERMINATION TARGET”. Since the assistant pulls the tissue with respective forceps held by two hands, there is an issue that the assistant is unable to notify the system of the completion of development of the surgical field with the assistant's forceps by expressing it by the movement of the forceps or the like in the images. To address this, the processor 110 performs determination like (3) or (3*), and can thereby determine whether the development of the surgical field with the assistant's forceps has been substantially completed.

(1*) (2*) The processor 110 uses the processing of detecting the treatment tool from the endoscope images, the processing of detecting gripping of the tissue with the treatment tool from the endoscope images, and processing of analyzing the deformation quantity of the tissue from the endoscope images for recognition in (1*) and (2*) in “DETERMINATION TARGET”. The deformation quantity is analyzed as follows. The processor 110 constantly analyzes the deformation quantity using an Optical Flow method based on AI such as RAFT, and recognizes that the tissue starts to move from the deformation quantity exceeding a threshold indicating the removal of slack in the tissue. For example, assume that the displacement of the analysis point and the strain of the cell are used as the deformation quantity. As illustrated in B1 in FIG. 10, in a case where the shape of a cell connecting four analysis points for analysis of deformation is strained in one direction like a cell 41b in comparison with a cell 41a in a previous frame, it is considered to be a deformation that removes the slack. As illustrated in B2, to detect the deformation, in a case where main strain with a main component in a pulling direction 30 exceeds the threshold, it can be determined that a scene is the pulling scene. Note that it is sufficient if the movement of the tissue is detected, so that relative strain or a displacement quantity and the threshold may be compared with each other. The method of analyzing the deformation is not limited to Optical Flow based on AI, and various kinds of other methods such as classical Optical Flow may be used.

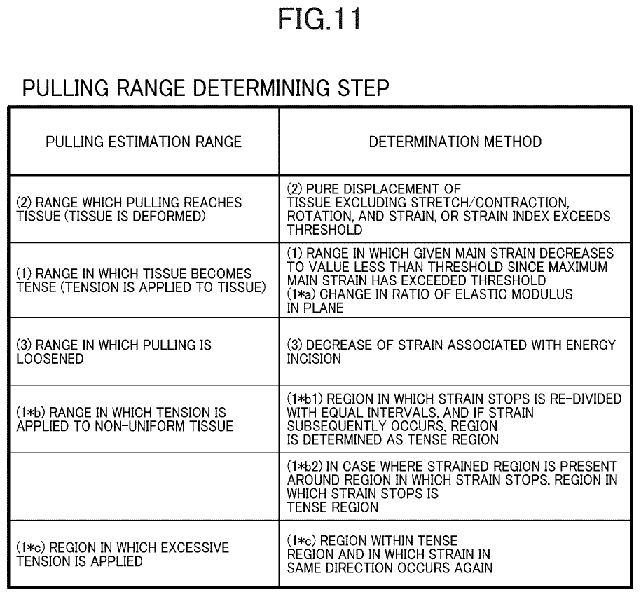

Details of a pulling range estimating step S23 are described. FIG. 11 is a table describing a pulling estimation range and a determination method in the pulling range estimating step S23. A bracketed number such as (1) represents an embodiment. An item to which an identical number is added in FIGS. 8 and 11 represents a corresponding embodiment. For example, (1) in FIG. 8 and (1) in FIG. 11 represent a corresponding embodiment. * means a modification. For example, (1) in FIG. 8 and (1*) in FIG. 11 may correspond to each other, and (1*) in FIG. 8 and (1) in FIG. 11 may correspond to each other. There is a case where an alphabet or the like is further added to *. This is merely for the purpose of convenience to distinguish items, and, for example, (1*a) or the like remains a modification of (1). For example, not only (1) and (1*) in FIG. 11, but also all modifications to which [1*] is added such as (1*a) may correspond to (1) and (1*) in FIG. 8.

### [Pulling Estimation Range]

(2) The processor 110 estimates a range in which the pulling reaches the tissue, that is, a range in which the tissue is deformed, as the pulling range.

(1) The processor 110 estimates a range in which the tissue becomes tense, that is, a range in which tension is applied to the tissue, as the pulling range.

(3) The processor 110 estimates a range in which the pulling is loosened, that is, a range in which deformation or the like of the tissue, which has been deformed by the pulling, is loosened, as the pulling range.

(1*b) In a case where a tissue whose response to the pulling is not uniform is mixed, the processor 110 uses a determination method, which will be described later, to estimate the range in which tension is applied to the tissue as the pulling range.

(1*c) The processor 110 estimates a range in which excessive tension is applied to the tissue as the pulling range.

### [Determination Method]

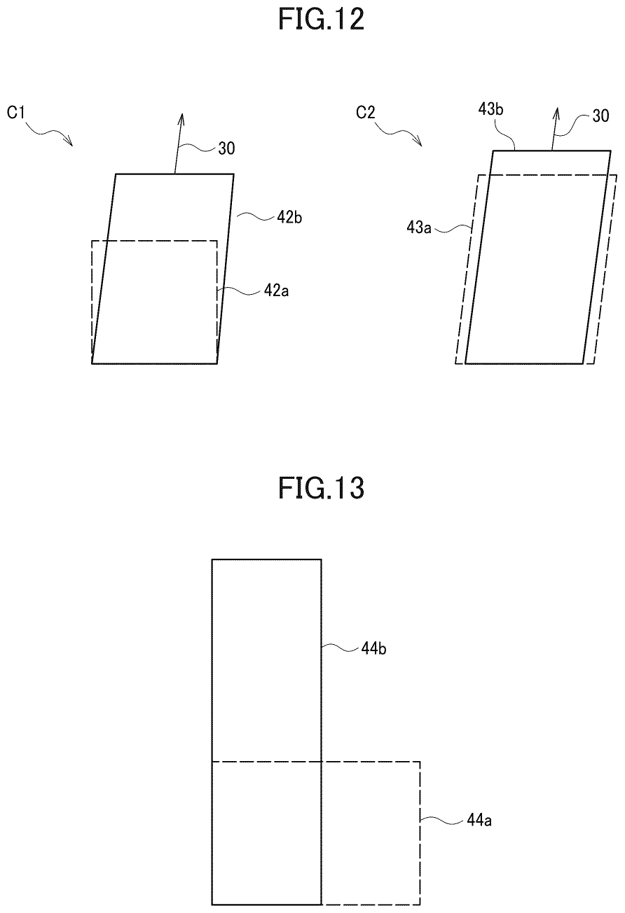

(1) In (1) in “PULLING ESTIMATION RANGE”, the processor 110 estimates a range in which given main strain decreases to a value less than a threshold since maximum main strain indicating strain in the pulling direction has exceeded the threshold due to the pulling with the forceps held by the left hand of the operator as the region in which tension is applied to the tissue. The given main strain is a minimum main strain in a case where an analyzed space is a two-dimensional space. Alternatively, in a case where the analyzed space is a three-dimensional space, the given main strain is another main strain in an appropriate two-dimensional plane including the maximum main strain, or main strain in a plane orthogonal to the maximum main strain, or second main strain. As illustrated in C1 in FIG. 12, the tissue stretches due to the pulling, whereby a cell 42a is deformed into a shape like a cell 42b. The cell 42b indicates a state where the main strain with the main component in the pulling direction 30 becomes large. Such a deformation occurs until the tissue is expand by the pulling. As illustrated in C2, a cell 43a becomes to have a shape like a cell 43b due to the pulling in a state where tension is applied to the tissue, but the deformation is small. In this state, the cell stretches in a pulling direction 30, while it contracts in a direction orthogonal to the pulling direction 30. That is, the maximum main strain representing the strain in the pulling direction 30 increases, while the main strain in the direction orthogonal to the pulling direction 30 decreases. The processor 110 makes determination about the above using a threshold. In the determination, it may be preferable that images be analyzed by excluding disturbance due to the camera. The disturbance is disturbance of the camera, such as scaling, rotation, translational movement, depth strain in viewing a solid in a 2D image. Note that only the maximum main strain may be used in the above-mentioned determination.

(1*a) In (1) in “PULLING ESTIMATION RANGE”, the processor 110 may use a change in a ratio of elastic modulus in a plane of the tissue to estimate the region in which tension is applied to the tissue. A relation of elastic modulus=stress/strain holds. That is, as illustrated in C2 in FIG. 12, since the elastic modulus increases in the region in which tension is applied, for example, the processor 110 is only required to detect that the elastic modulus exceeds the threshold.

(2) In (2) in [PULLING ESTIMATION RANGE], the processor 110 analyzes the deformation of the tissue to detect the range which the pulling by the assistant's forceps reaches. For example, an analysis method similar to that in FIG. 10 may be adopted. That is, in a case where the main strain with the main component in the pulling direction 30 exceeds the threshold, it can be determined that the cell is included in the pulling range. Note that relative strain or a displacement quantity and the threshold may be compared with each other. In a case where the forceps are not constantly seen in the field of view, that is, in a case where the forceps are framed out, a point of the maximum displacement near a position at which the forceps have been seen the last time may be re-defined as a virtual pulling position and used for analysis of the pulling direction, or the forceps may be disposed by estimation at a coordinate position outside the screen and used for measurement.

(3) As described in steps S3 and S4 in FIG. 1, when a new surface appears in the tissue by energy incision, there is a possibility that loosening occurs in the tissue if the tissue remains to be pulled by the assistant's forceps in the same state. For this reason, a range in which the pulling is loosened as described in (3) in “PULLING ESTIMATION RANGE”. Specifically, the processor 110 uses a result of analysis of deformation in several seconds in the past to analyze a difference in deformation quantity, and estimates the range in which the pulling is loosened based on the difference. An example in which the deformation quantity is the main strain is described here. The processor 110 estimates a range in which the main strain in cells of the evaluation mesh changes in a tissue-contraction direction as a region with a possibility for contraction of the tissue. Specifically, the processor 110 uses the main strain in the cell at a timing at which the energy treatment tool comes in contact with the tissue as a criterion to calculate a decrease quantity of the main strain in the cells at each of subsequent timings. In a case where the decrease amount of the main strain falls below the threshold, the processor 110 estimates a range including the cells as the range in which the pulling is loosened. Note that the surface that newly appears since the start of analysis due to incision or the like may be ignored in analysis of the displacement quantity. Alternatively, an evaluation mesh is newly added to the surface that newly appears, and the analysis of deformation may be executed from this time point.

(1*c) If the pulling is too strong, the tissue ruptures. Although (1) in assistance for the operator can also be a target, in robotic surgery without any haptic sense, there are similar concerns in cases of the assistant's forceps or a third arm on which attention is hard to be paid. Hence, by detecting the region in which excessive tension is applied like (1*c) in “PULLING ESTIMATION RANGE”, it is possible to separately present the region before a rupture for safety. Specifically, an object succumbs to stress before the rupture. The processor 110 detects succumbing based on the deformation quantity of the cells. An example in which the deformation quantity is the main strain is described here. By the above-mentioned methods of various kinds, the region in which the tissue becomes tense and tension is applied to the tissue is defined. In a case of detecting that the main strain in the pulling direction has increased by a certain quantity or more in the region, the processor 110 estimates the region as the region in which excessive tension is applied. This is because it is estimated that the object succumbs to stress, and is about to rupture in the region. As illustrated in FIG. 13, a cell 44a is not deformed much in the region in which tension is applied to the tissue, but when the tissue is further pulled, the main strain becomes large at some point like a cell 44b. The processor 110 makes determination about this using a threshold.

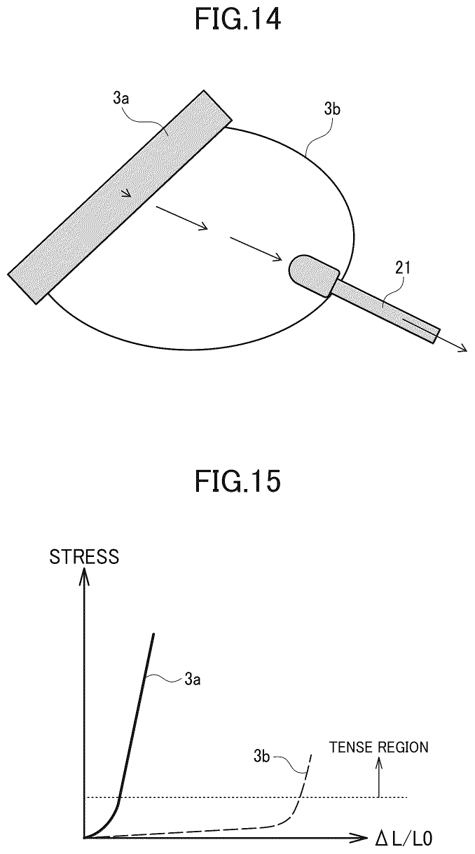

(1*b1) A consideration will be given to an issue in a case where a wide range of the inside of the body is seen in the field of view of the endoscope, and the operator or the assistant continuously pulls a plurality of tissues whose manners of deformation are different. An example in FIG. 14 is used here, and assume that the deformation is stretch. As illustrated in FIG. 14, a hardly stretchable tissue 3a and an easily stretchable tissue 3b are connected with each other, and are pulled from the easily stretchable tissue 3b side with the forceps 21. In a case where only the slack in the tissues 3a and 3b is removed, stress does not occur anywhere in the tissues 3a and 3b. However, when stretch in a tissue somewhere reaches a limit, a major part of the pulling quantity is generated in the easily stretchable tissue 3b, and the hardly stretchable tissue 3a hardly stretches. When a consideration is given in one dimension for easier understanding, equal pulling force is applied to the tissues 3a and 3b on an axis. Hence, it is estimated that stress (tension), which is generated in the pulling range, is largely generated in the hardly stretchable tissue 3a.

FIG. 15 illustrates a relationship between stress and a deformation quantity of each of the hardly stretchable tissue 3a and the easily stretchable tissue 3b. An example in which ΔL/L0 is the deformation quantity is described here. It is understood that ΔL/L0, that is, the pulling quantity, until reaching the tense region, is larger in the easily stretchable tissue 3b than in the hardly stretchable tissue 3a.

In this manner, in a case where the pulling is detected in a range that cannot be approximated as a uniform tissue, if determination is simply made based only on a constant threshold for the deformation quantity, only the easily stretchable tissue 3b is detected by the determination using the threshold, and this region is presented to the user. That is, since the user looks only at the easily stretchable tissue as a presentation range, attention may be required. When a tissue that is hard to be stretched and in which stress quickly rises with respect to the deformation such as a thick connective tissue is tried to be displayed by way of thinking similar to that in a case of a tissue that is not the above-mentioned tissue, it is inadvisable to provide a constant threshold for the deformation quantity.

To address this, (1*b) in “PULLING ESTIMATION RANGE”, the processor 110 performs analysis of the deformation again, that is, re-divides the region. Specifically, in a case where a region in which a change in component of the main strain in the pulling direction becomes small appears due to the pulling, the processor 110 re-divides the region with the evaluation mesh with equal intervals and continues the analysis of deformation. Thus, since the processor 110 resumes the analysis at a point at which stress with respect to a strain is about to rise, a small strain means generation of large stress within the re-divided range. Hence, after the region is re-divided, in a case where the strain is generated within the region, the region is estimated as the region in which tension is applied.

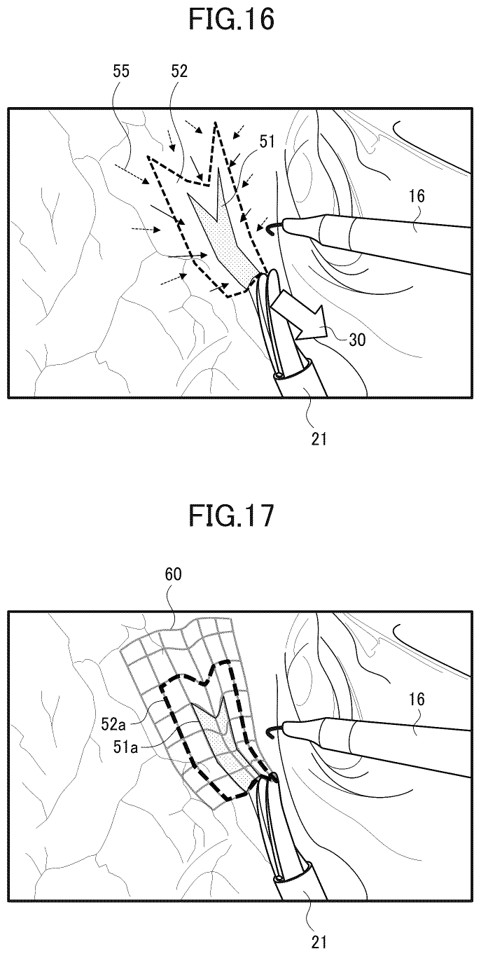

(1*b2) A consideration will be given of an issue in a case where a wide range of the inside of the body is seen in the field of view of the endoscope, and the tissue is uniform but a range in which tension is generated by the pulling is limited depending on a degree of physiological adhesion or the like. An example in FIG. 16 is used here. The tissue, the operator or assistant's forceps 21, and the operator's energy treatment tool 16 are seen in an endoscope image. The tissue is pulled in the pulling direction 30 with the forceps 21.

A biological tissue includes a certain buffer region with respect to deformation such as stretch and strain, and has a characteristic in that stress does not rise immediately after the biological tissue is pulled. Hence, as a result of the pulling, tension is generated in a region 51 in which the deformation does not occur any more. Assume that the deformation is stretch here. There is a simply stretching region 52 in the surroundings of the region 51 in which tension is generated. An arrow 55 added to the surroundings of the region 52 indicates a direction of the stretch of the tissue. The tissue further outside the region 52 makes translational movement or does not move with respect to the pulling.

In the above-mentioned modification (1*b1), the region is re-divided after the stretch of the tissue stops. However, since the stretch of the tissue after re-division is very small, there is a possibility that the stretch is equal to or less than a limit for the detection of the deformation quantity. For this reason, there is a possibility that a signal-to-noise (S/N) ratio in the estimation of the pulling range deteriorates. Furthermore, it is difficult to determine whether the stretch of the tissue stops simply because the pulling stops, or because tension is applied. Here, the stop may include when the stretch of the tissue is below detection limit or the stretch of the tissue becomes small.

To address this, in (1*b) in “PULLING ESTIMATION RANGE”, in a case where there is the region 52 that surrounds the region 51 in which the stretch of the tissue stops and that simply stretches around the region 51, the processor 110 estimates that the region 51 in which the stretch stops as the region in which tension is applied. This eliminates the need for detection of minute displacement and can increase the S/N ratio in detection. The method of estimating the deformation quantity of each cell in the evaluation mesh is as described above. In a case where the deformation quantity of cells becomes a certain value or more and the deformation of the cells stops, the processor 110 determines that the hand simply stops if the deformation of the whole of the evaluation mesh stops. In a case where cells in the surroundings of the cells in which the deformation stops continue to deform, the processor 110 estimates that tension is applied to the region including the cells that stop to deform. Further details are now described with FIGS. 17 to 20. In the following example, assume that the deformation quantity is main strain.

As illustrated in FIG. 17, the processor 110 sets an evaluation mesh 60 in endoscope images, and calculates main strain in each cell at each time t (n, n−1, . . . ). The processor 110 detects cells in which the main strain is a threshold or more and a change in main strain is small, and sets the cells as a candidate region 51a. The threshold is, for example, +25% to +60%, but is not limited thereto. The change in main strain being small means that an average of time is ±5%/0.5 sec., but is not limited thereto. The processor 110 extends a region to the surroundings of the candidate region 51a, and sets the region as a comparison region 52a. For example, the processor 110 sets 50 pixels in the surroundings of the candidate region 51a as the comparison region 52a.

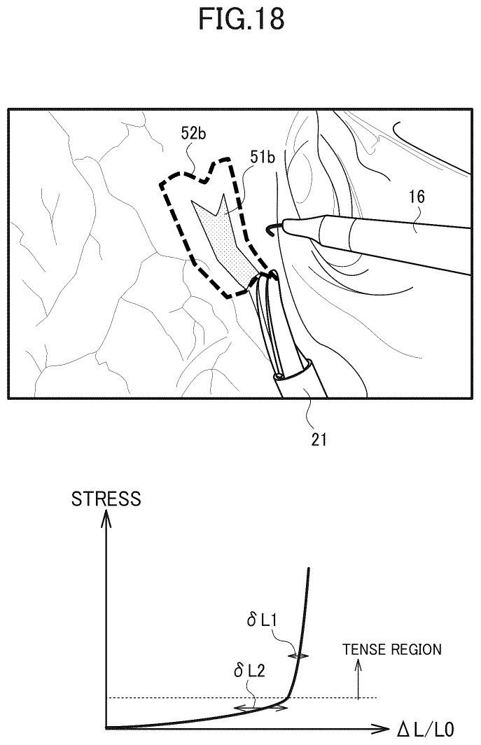

A lower graph in FIG. 18 illustrates a relationship between a deformation quantity of the tissue and stress. In this graph, an abscissa axis indicates ΔL/L0. Since each of ΔL/L0 and the main strain in each cell is considered to be generated in the direction of the stretch of the tissue, a description is given on the assumption that ΔL/L0 and the main strain are correlated. Note that it is considered that the direction of the stretch of the tissue is directed to the pulling direction by the forceps in the candidate region, and the direction of the stretch of the tissue is directed to the candidate region in the comparison region. As illustrated in the lower graph in FIG. 18, in a case where a time change δL2 of the main strain in the comparison region with respect to a time change δL1 of the main strain in the candidate region is a predetermined magnification or more, or in a case where a difference between δL1 and δL2 is a predetermined difference or more, the processor 110 determines that tension is applied in the candidate region. The predetermined magnification is, for example, a three-fold magnification. In a case of determining that tension is applied in the candidate region, the processor 110 presents the candidate region as a tense region 51b. For example, the processor 110 adds a color such as light blue to the tense region 51b and superimposes the tense region 51b on the endoscope image, and displays the endoscope image on the monitor. The processor 110 may further present an extension range with a dotted line 52b or the like.

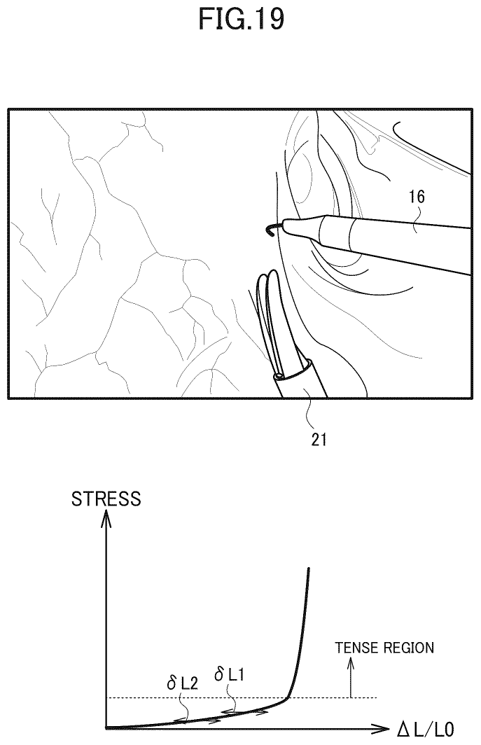

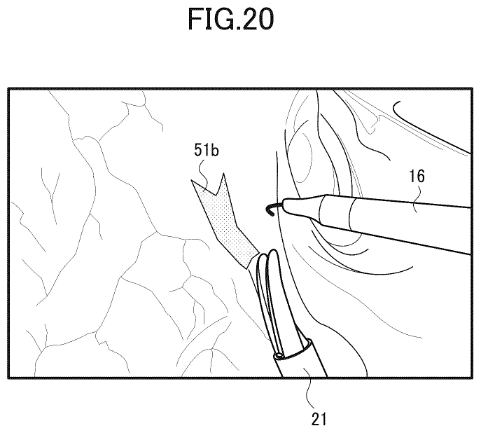

As illustrated in FIG. 19, in a case where the time change δL2 of the main strain in the comparison region with respect to the time change δL1 of the main strain in the candidate region is less than the predetermined magnification, or in a case where the difference between δL1 and δL2 is less than the predetermined difference, the processor 110 estimates that tension in the candidate region is not sufficient and the pulling merely stops. In this case, the processor 110 does not present the candidate region as the tense region.

As illustrated in FIG. 20, in a case of estimating that there is the tense region 51b in images once, the processor 110 maintains presentation of the tense region 51b even if a subsequent change in the main strain of the tense region 51b over time is small. For example, the processor 110 maintains presentation of the tense region 51b after estimating that there is the tense region 51b in the images once until detecting the loosening of the tissue.

Various methods of displaying a region may be employed. For example, the region may be displayed by bordering, filling, or a lighting change. The bordering may be added only to an outer rim portion of the candidate region, may be added to an outer periphery of the candidate region excluding a gripping portion, or may be added only to the gripping portion to a furthermost end in the candidate region. The filling may be translucent, superposition of a pattern, or a change in density depending on strength of the pulling. The lighting change may be lighting, blinking, gradual decrease of brightness, or extinguishing of light over time. Additionally, display as described in the following presentation information processing step S25 may be performed.

Details of the presentation information processing step S25 are now described. The processor 110 outputs at least a result of analysis of the pulling range as it is or performs post-processing and outputs at least the result of analysis of the pulling range. Image information, coordinate information, or a label on information processing for display may be output.

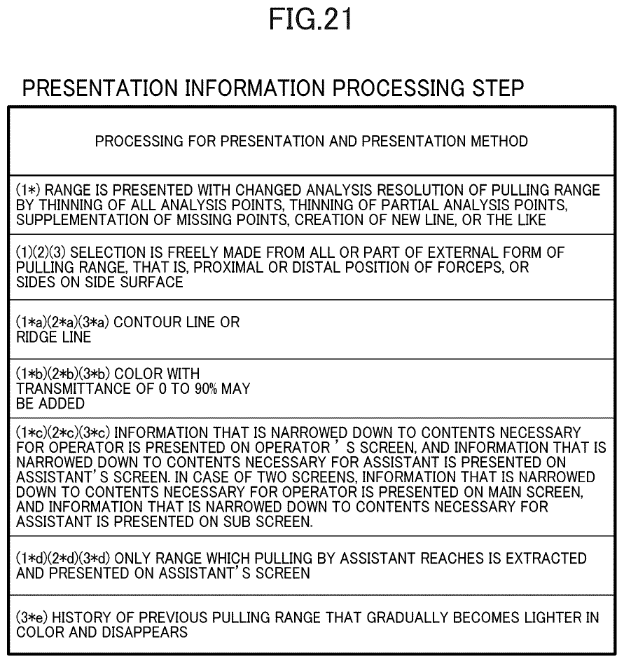

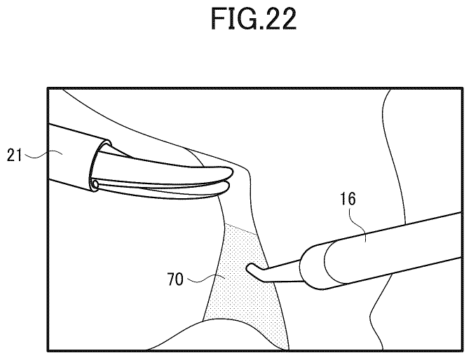

FIG. 21 is a table describing processing for presentation and a presentation method in the presentation information processing step S25. A bracketed number such as (1) represents an embodiment, and * represents a modification. A correspondence relation between an embodiment and a modification among FIGS. 8, 11, and 21 is as described with reference to FIG. 11. Each item in FIG. 21 is described below.

(1*) The processor 110 presents all analysis points in the pulling range. Alternatively, the processor 110 may change resolution of analysis points in the pulling range to present the pulling range. Examples of the change of resolution include thinning of partial analysis points, supplementation of missing points among analysis points, or creation of a new line in the evaluation mesh.

(1)(2)(3) The processor 110 presents the whole of an external form of the pulling range. Alternatively, the processor 110 may freely select and present part of the external form of the pulling range. Examples of the part of the external form include a portion of the external form proximal or distal to the forceps, and a side on a side surface of the external form.

(1*a)(2*a)(3*a) The processor 110 presents a contour line or a ridge line indicating the deformation quantity of the pulling range, or a line similar to the contour line or the ridge line.

(1*b)(2*b)(3*b) The processor 110 may add a color to the pulling range. The color may be added with transmittance of 0 to 90%. An identical color or identical transmittance may be added to the whole of the pulling range, or a different color or different transmission rate may be added to each cell.

(1*c)(2*c)(3*c) The processor 110 presents information that narrows down to contents necessary for the operator on a screen seen by the operator. Additionally, the processor 110 presents information that narrows down to contents necessary for the assistant on a screen seen by the assistant. In a case where the medical system includes two monitors, for example, the information for the operator may be displayed on the main monitor and the information for the assistant may be displayed on the sub monitor.

(1*d)(2*d)(3*d) The processor 110 extracts only a range which the pulling with the assistant's forceps reaches, and presents the range on the screen seen by the assistant.

(3*e) The processor 110 presents a history of a previous pulling range that gradually becomes lighter in color and disappears. That is, the processor 110 makes display of a color, a line, or the like lighter in color with respect to information regarding an older pulling range.

FIGS. 22 to 35 illustrate examples of the respective presentation methods described above. Note that an example in which the treatment tool such as the forceps is seen in endoscope images is used, but the treatment tool is not necessarily seen in the images.

FIG. 22 illustrates specific examples of (1), (2), (3), (1*b), (2*b), and (3*b). The tissue, the operator or assistant's forceps 21, and the operator's energy treatment tool 16 are seen in an endoscope image. The processor 110 superimposes the range estimated as the pulling range, out of the range in which the evaluation mesh is set when the pulling range is analyzed, on endoscope images, and thereby outputs the pulling range as image information in real time. The processor 110 highlights peripheral sides of a frame, mainly a distal frame of the pulling range of the pulling by the operator or the assistant, fills the inside of the sides with a translucent color, outputs the pulling range on the monitor to present the pulling range to the operator or the assistant.

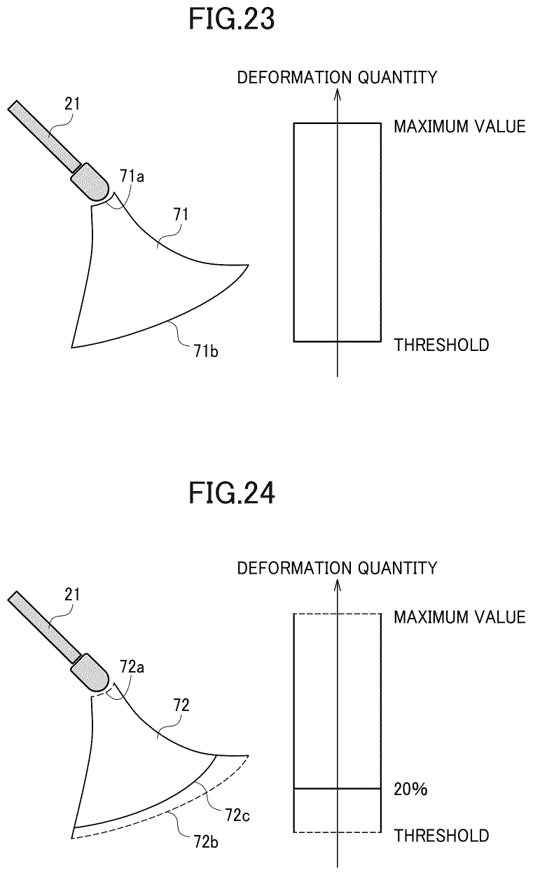

FIG. 23 illustrates specific examples of (1), (2), and (3). The processor 110 displays an outer frame of a region 71 in which the deformation quantity exceeds the threshold within the evaluation region in which the evaluation mesh is set. The deformation quantity is a maximum value at a side 71a proximal to the forceps 21, and is the threshold at a side 71b distal to the forceps 21.

FIG. 24 illustrates specific examples of (1), (2), (3), (1*a), (2*a), and (3*a). The processor 110 displays a line 72c that connects cells whose deformation quantity is a predetermined value within the evaluation region in which the evaluation mesh is set. The line 72c corresponds to the contour line of the deformation quantity. FIG. 24 illustrates an example in which the predetermined value is 20%, but the predetermined value is only required to be larger than the threshold and smaller than the maximum value. The outer frame of the region 72 in which the deformation quantity exceeds the threshold may or may not be displayed. Alternatively, only a side line out of the outer frame of the region 72 may be displayed and the side 72a whose deformation quantity is the maximum value and the side 72b whose maximum value is the threshold may not be displayed. Still alternatively, the line 72c, the side 72a, and the side 72b may be displayed using different types of lines such as a solid line and a dotted line.

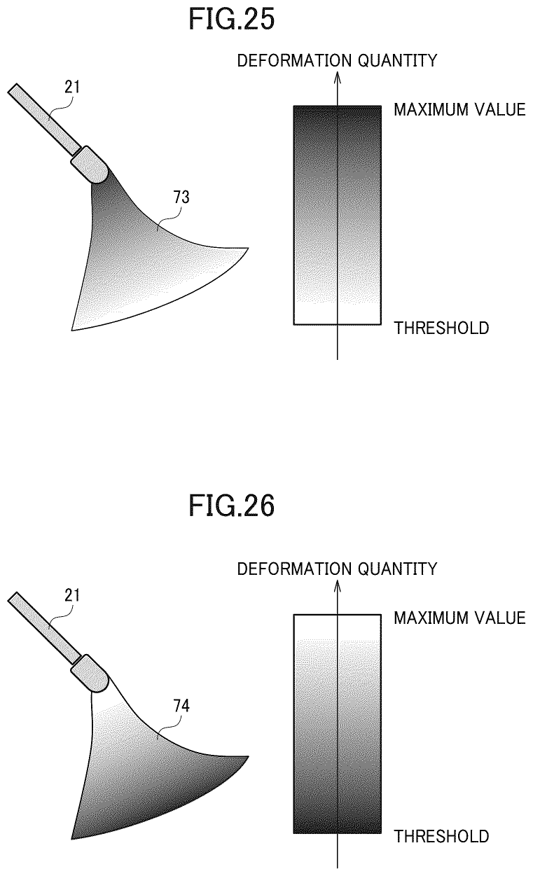

FIGS. 25 and 26 illustrate specific examples of (1*b), (2*b), and (3*b). The processor 110 adds gradation colors to regions 73 and 74 in which the respective deformation quantities exceed the threshold within the evaluation region in which the evaluation mesh is set. The color density of gradation is determined depending on the deformation quantity, and may be defined by, for example, a sigmoid function with respect to the deformation quantity. FIG. 25 illustrates an example in which a color becomes lighter from the maximum value of the deformation quantity to the threshold in the region 73, that is, the color becomes lighter from the proximal end of the forceps 21 to the distal end of the forceps 21. FIG. 26 illustrates an example in which a color becomes darker from the maximum value of the deformation quantity to the threshold in the region 74, that is, the color becomes darker from the proximal end of the forceps 21 to the distal end of the forceps 21. In FIG. 26, how far the pulling reaches is easily visually recognized.

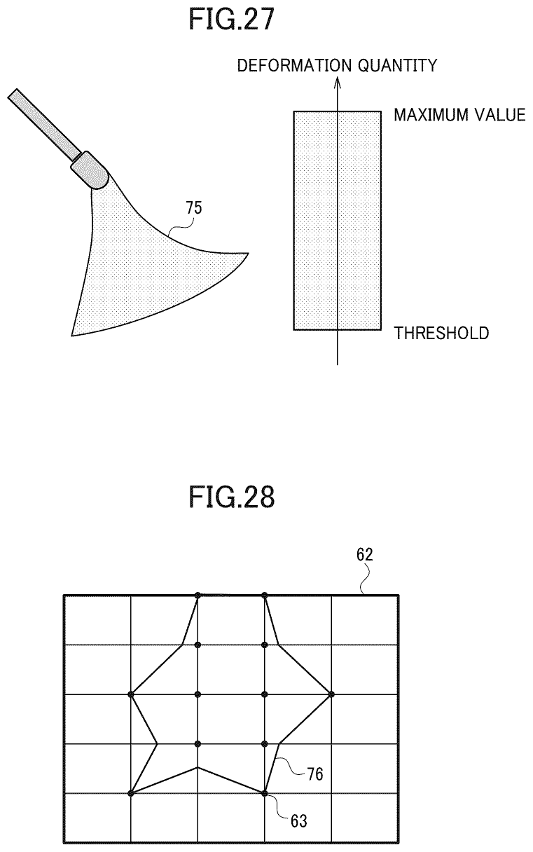

FIG. 27 illustrates specific examples of (1*b), (2*b), and (3*b). The processor 110 fills a region 75 with a color with certain transmittance. The region 75 is a region in which the deformation quantity exceeds the threshold within the evaluation region in which the evaluation mesh is set.

FIG. 28 illustrates a specific example of (1*). FIG. 28 illustrates an analysis point whose deformation quantity exceeds the threshold with a black circle 63. When black circles 63 are merely displayed, or cells whose four corners are black circles 63 are merely filled with a color and displayed, there is a possibility that resolution for presentation of the region is insufficient. To address this, the processor 110 uses a known method such as a median point to increase resolution for presentation of the region. The processor 110 uses a known algorithm to change resolution of the black circles 63, and estimate and present an extended pulling range 76. The processor 110 may display the external form of the pulling range 76, or may fill the pulling range 76 with a color and display the pulling range 76.

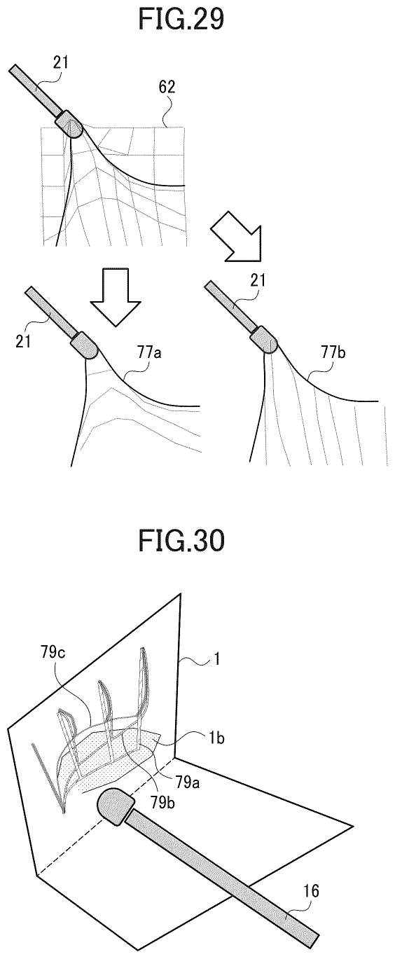

FIG. 29 illustrates specific examples of (1*a), (2*a), and (3*a). When the evaluation mesh 62 itself is displayed, there is a possibility for information overload. To address this, the processor 110 leaves a longitude line like 77a out of a range in which the deformation quantity exceeds the threshold in an evaluation mesh 62 to perform display similar to display using the contour line. Alternatively, the processor 110 leaves a latitude line like 77b out of the range whose deformation quantity exceeds the threshold in the evaluation mesh 62 to perform display similar to display using the ridge line. The processor 110 may display the longitude line or the latitude line that is approximated by a curve line. By performing such display, a quantity of information decreases so as to provide information necessary for the operator, and the inside of the presentation region becomes simple. As a result, it is possible to display important dissection without being hidden by the presentation information.

FIG. 30 illustrates a specific example of (3*e). When a tissue 1 is incised with the energy treatment tool 16, and the new surface 1b appears in the tissue 1, loosening occurs in the tissue 1. At this time, the processor 110 presents displays 79a, 79b, and 79c of the pulling range that disappear over time as history information. The display 79c is the newest and the darkest in color, the display 79b is the second newest and the second darkest in color, and the display 79a is the oldest and the lightest in color. By presenting such displays, it is possible to constantly make a comparison with the past especially for giving notice of loosening of the tissue.

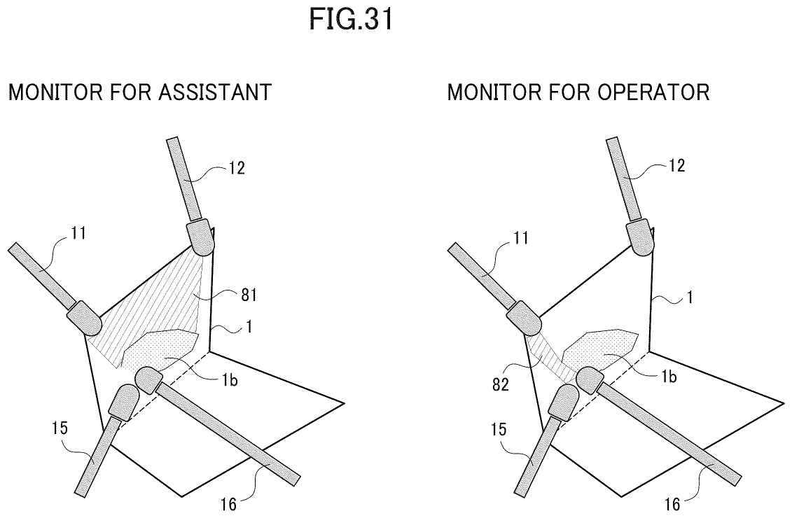

FIG. 31 illustrates specific examples of (1*c), (2*c), (3*c), (1*d), (2*d), and (3*d). The processor 110 may present information only on the monitor for the assistant as an assistant-assistance function. For example, the processor 110 may display a range 81 which the pulling with the assistant's forceps 11 and 12 reaches on the monitor for the assistant. The processor 110 may present information only on the monitor for the operator as an operator-assistance function. For example, the processor 110 may display a range 82 which the pulling with the operator's forceps 15 reaches on the monitor for the assistant. Additionally, in a case where both the assistant-assistance function and the operator-assistance function are used, the processor 110 may present information for the assistant on the monitor for the assistant and present information for the operator on the monitor for the operator. Furthermore, there is a possibility that the information for the assistant is utilized by an experienced operator for giving an instruction. In a case where the assistant is more experienced than the operator, there is also a possibility that the assistant gives an instruction to the operator. Thus, any information may be changed in appearance and displayed on an identical screen.

Especially, regarding assistance for presenting loosening of the pulling, that is, regarding (3*c) and (3*d), conceivable specific examples are as follows. As the loosening of the pulling due to the energy treatment tool, there is a possibility for occurrence of loosening in a range in which loosening can be resolved only by the assistant's forceps 11 and 12 and loosening in a range in which loosening should be resolved in cooperation with the operator's forceps 15 or by the operator himself/herself. At this time, presentation of information regarding loosening that can be resolved only by the operator on the monitor for the assistant is meaningless unless used for coaching. To address this, in assistance for presenting the loosening of the pulling, the processor 110 may present information only on the monitor for the assistant in a case where the pulling by the assistant is necessary, and may present information only on the monitor for the operator in a case where the pulling by the operator is necessary. That is, only information regarding a range 81 in which loosening can be resolved by the assistant's forceps 11 and 12 may be presented on the monitor for the assistant. Additionally, only information regarding a range 82 in which loosening can be resolved by the operator's forceps 15 may be presented on the monitor for the operator. Alternatively, information may be merely presented by labeling, color coding, or the like so as to make which of the operator or the assistant can address loosening identified. Note that it is conceivable that the estimation of the range is roughly defined by, for example, a positional relationship of the forceps 11 and 12, a positional relationship of the forceps 15, or a positional relationship of the forceps 11, 12, and 15.

Note that the method of presenting information is not limited to the methods described with reference to FIGS. 21 to 31.

The processor 110 may enclose an outer frame of an analysis range, or may use an analyzed analysis point itself. Additionally, the processor 110 may narrow down to a range in which certain tension is applied out of the analysis range, or may change presentation depending on the magnitude of tension. Alternatively, it is also conceivable that the processor 110 focuses only on a highly tense region, and presents the region as an alert.

Additionally, from another viewpoint, as a result of estimation of the pulling region by analysis of the deformation quantity of the tissue, the processor 110 may especially highlight a dissection structure that is within the pulling region and that characteristically indicates the pulling, that is, a sulcus, a ridge line, a dent, muscle, a loose connective tissue that stands out, or the like to perform display. A machine learning method may be separately used for detection of the structure. Additionally, when there is a difference in followability of analysis points in the analysis of the deformation quantity of the tissue, there is a case where an important dissection structure is hidden in an invisible deep portion within the pulling region. Thus, the processor 110 may highlight a region with low followability of analysis points to perform display so that the region can be identified.

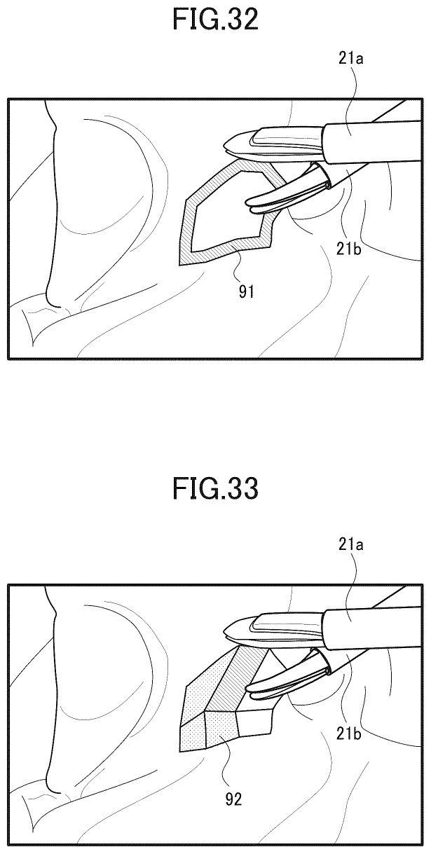

As illustrated in FIG. 32, the processor 110 may display a region in which the tissue is deformed by a certain quantity or more due to the pulling with a frame 91 or the like since when the tissue is gripped with forceps 21a or forceps 21b as a starting point.

As illustrated in FIG. 33, the processor 110 may display the direction and intensity of deformation in the region in which the tissue is deformed by the certain quantity or more due to the pulling since when the tissue is gripped with the forceps 21a or the forceps 21b as the starting point. For example, the processor 110 may display, in each cell of an evaluation mesh 92, a color indicating the direction and intensity of the deformation of the cell. For example, red may indicate tension, blue may indicate loosening, and the density of each color may indicate the intensity of deformation.

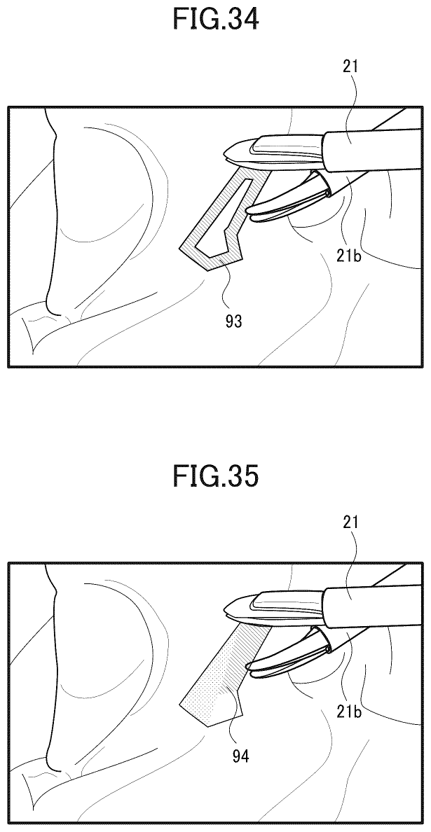

As illustrated in FIG. 34, the processor 110 may display a region in which certain tension is applied to the tissue due to the pulling with a frame 93 or the like since when the tissue is gripped with the forceps 21a or the forceps 21b as the starting point.

As illustrated in FIG. 35, the processor 110 may display intensity distribution 94 regarding tension that is generated in the tissue due to the pulling since when the tissue is gripped with the forceps 21a or the forceps 21b as the starting point. The intensity distribution 94 may be, for example, displayed by color coding in three steps of low, medium, and high, or in color density of three steps.

In the present embodiment, the image processing system 100 includes the processor 110. The processor 110 sequentially acquires time-series images captured by the endoscope 500 and performs deformation analysis processing on the time-series images. The processor 110 disposes an evaluation mesh including a plurality of analysis points in a freely-selected timing image out of the time-series images in the deformation analysis processing. The processor 110 deforms the evaluation mesh in each image of the time-series images so that each analysis point in each image of the time-series images tracks a characteristic point of an object located on each analysis point in the freely-selected timing image in which the evaluation mesh is disposed. The processor 110 calculates the deformation quantity of each cell in the evaluation mesh based on the magnitude and the direction of the movement quantity of each analysis point in each image. The processor 110 presents information regarding deformation of the evaluation mesh based on the calculated deformation quantity. Assume that the movement quantity of the analysis point mentioned herein is a vector quantity, and includes magnitude and a direction.

In accordance with the present embodiment, the processor 110 tracks the deformation of the object with the evaluation mesh, and presents information regarding the deformation of the evaluation mesh based on the deformation quantity. As described with reference to FIGS. 2 and 3 and the like, there is a relationship between the deformation of the tissue and the pulling state. This allows the user to determine the pulling state of the tissue by visually recognizing information regarding the deformation of the object presented by the system. Additionally, in the present embodiment, the deformation is tracked by image recognition and presented, thereby eliminating the need for sensing of force by a force sensor or the like. Furthermore, since it is the deformation that is tracked, it is possible to perform tracking processing based on a rule, or there is no need for information regarding sensing of force in a training phase even in a case where the deformation is tracked using a machine learning model.

In the present embodiment, the processor 110 may superimpose a display in a mode depending on the deformation quantity in each image of the time-series images on each image. For example, the processor 110 may superimpose a display in which each cell is colored depending on the deformation quantity of each cell, on each image.

In accordance with the present embodiment, the display in the mode depending on the deformation quantity is superimposed on an endoscope image, which allows the user to know the deformation quantity of each portion of the tissue by seeing the display. By knowing the deformation quantity of each portion of the tissue, the user can determine the pulling state of the tissue.

Additionally, in the present embodiment, the processor 110 may present the time-series images on the first monitor. The processor 110 may present the time-series images and the information regarding the deformation of the evaluation mesh on the second monitor.

In accordance with the present embodiment, displaying the endoscope images on which information for assisting the pulling is not superimposed on the first monitor ensures visibility of the endoscope images, while displaying the endoscope images on which the information for assisting the pulling is superimposed on the second monitor enables assistance for the user's pulling.

Additionally, in the present embodiment, the processor 110 may determine whether or not a scene is a pulling scene in which the object is pulled with the treatment tool based on the time-series images or the user's input. When determining that the scene is the pulling scene, the processor 110 may perform the deformation analysis processing on the evaluation mesh to analyze the deformation of the object due to the pulling.

In accordance with the present embodiment, it is possible to perform the deformation analysis processing on the evaluation mesh in a scene in which the user needs the pulling information and present the pulling information based on a result of the deformation analysis processing to the user.

In the present embodiment, the processor 110 may determine an object region in which tension is applied by the pulling based on the deformation quantity of each cell in each image of the time-series images and superimpose a display of the determined region on each image. Note that “(1) RANGE IN WHICH TISSUE BECOMES TENSE (TENSION IS APPLIED TO TISSUE)” in FIG. 11 corresponds to the object region to which tension is applied by the pulling. Note that, the object region in which tension is applied is only required to be displayed, and the evaluation mesh itself may or may not be displayed.

As described with reference to FIGS. 2 and 3, there is a relationship among the pulling of the tissue, the deformation of the tissue 2, and tension applied to the tissue. In the present embodiment, by utilizing this relationship, it is possible to present the pulling information to the user without directly detecting force applied to the tissue.

Additionally, the present embodiment may be implemented as an image processing method. The image processing method includes a step of sequentially acquiring time-series images captured by an endoscope and a step of disposing an evaluation mesh including a plurality of analysis points in a freely-selected timing image out of the time-series images. The image processing method includes a step of deforming the evaluation mesh in each image of the time-series images so that each analysis point in each image tracks a characteristic point of an object located on each analysis point in the freely-selected timing image in which the evaluation mesh is disposed. The image processing method includes a step of calculating a deformation quantity of each cell in the evaluation mesh based on the magnitude and direction of the movement quantity of each analysis point in each image, and a step of presenting information regarding the deformation of the evaluation mesh based on the calculated deformation quantity. The image processing method may be executed by a computer.

Additionally, the present embodiment may be implemented as a non-transitory information storage medium that stores a program. The program causes the computer to execute a step of sequentially acquiring time-series images captured by an endoscope and a step of disposing an evaluation mesh including a plurality of analysis points in a freely-selected timing image out of the time-series images. The program causes the computer to execute a step of deforming the evaluation mesh in each image of the time-series images so that each analysis point in each image tracks a characteristic point of an object located on each analysis point in the freely-selected timing image in which the evaluation mesh is disposed. The program causes the computer to execute a step of calculating a deformation quantity of each cell in the evaluation mesh based on the magnitude and direction of the movement quantity of each analysis point in each image, and a step of presenting information regarding the deformation of the evaluation mesh based on the calculated deformation quantity.

### 3. Second Embodiment

Also in the present embodiment similarly to the first embodiment, the image processing system 100 sets an evaluation region, recognizes pulling information regarding a tissue within the evaluation region, and presents the recognized pulling information to a user. The image processing system 100 sections the evaluation region with an evaluation mesh to perform analysis in recognition of a pulling state.

There is the following issue in the recognition of the pulling state. Note that, various modes in the second embodiment will be disclosed, but each mode may be the one that resolves part of the following issue.

(1) In terms of functional restrictions, in a case where the evaluation region strides a pulled tissue and the background, there is a possibility that the accurate deformation of the tissue cannot be analyzed or presented.

(2) In terms of usability, there is a possibility that unnecessary information presentation to a wide range leads to an obstacle to a physician's field of view.

(3) In terms of efficiency, in a case where recognition processing is performed in a wide range, there is a possibility for an enormous amount of calculation and longer processing time.

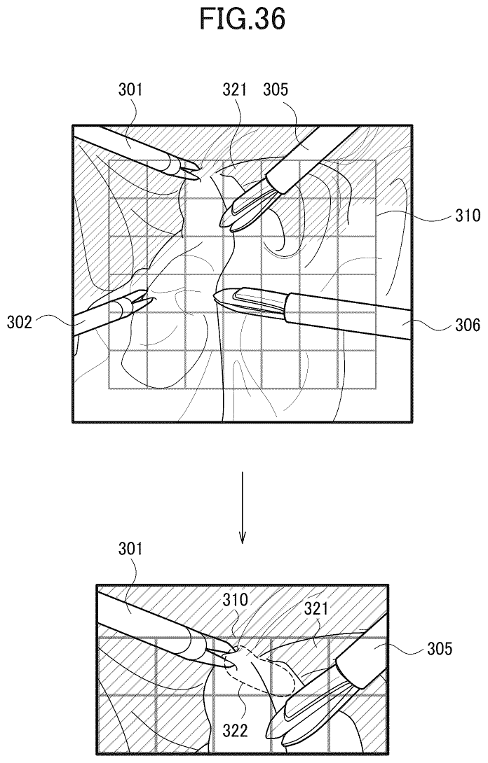

A supplementary description will be given of the above-mentioned (1) using an example in FIG. 36. As described in an upper view in FIG. 36, an evaluation mesh 310 is set in a region in which a treatment is performed with forceps 301, 302, 305, and 306, and the region includes a background 321. The background 321 mentioned herein is not the treatment target tissue, but mainly a tissue on a rear side of the treatment target in the field of view. That is, the background 321 is a region in which there is no influence of the pulling with the forceps. A lower view in FIG. 36 is a partial enlarged view of the upper view. As illustrated in the lower view, a region in which the evaluation mesh 310 is set includes a tissue region 322 and the background 321. The tissue region 322 is moved by the pulling with the forceps 301 reaching thereto. The pulling does not reach the background 321, and the background 321 does not move. In analysis of deformation on the evaluation mesh 310, there is a possibility that the pulling state of the tissue region 322 cannot be analyzed accurately due to the influence of the background 321 that has no relevance to the pulling. More specifically, there is a case where one cell includes the tissue region 322 and the background 321. The tissue region 322 is moved by the pulling reaching thereto. The pulling does not reach the background 321, and the background 321 does not move. In such a case, an analysis point that corresponds to and follows the movement of the tissue region 322, and an analysis point that corresponds to the stationary background 321 and that does not follow the pulling exist, and there is a possibility that the deformation of the evaluation mesh 310 becomes inadequate.

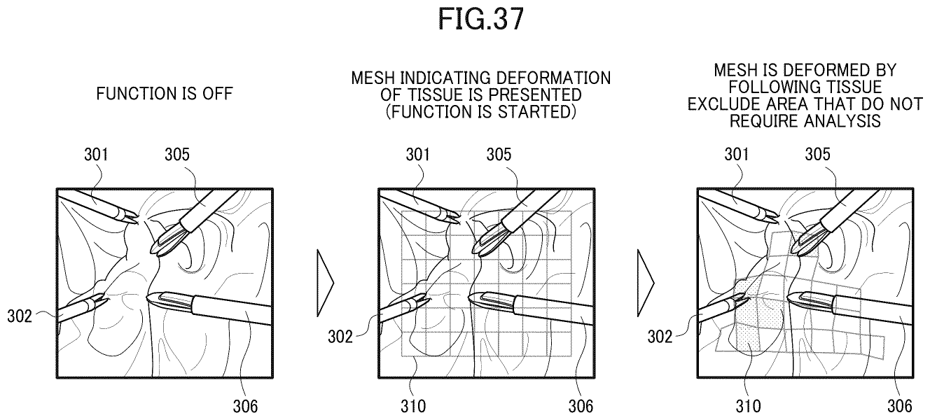

To address the issues of (1) to (3) described above, the image processing system 100 starts analysis in a wide range as the evaluation region, and gradually narrows the evaluation region down to a necessary region. As illustrated in FIG. 37, a description will be given below of an example in which, when a function starts from an OFF state, the image processing system 100 presents the evaluation mesh 310 indicating the deformation of the tissue on endoscope images. However, the image processing system 100 may define the evaluation mesh 310 on the background after the start of the function, and present the evaluation mesh 310 on the endoscope images at an appropriate timing. The image processing system 100, for example, sets a region that is operated by the assistant's forceps 301 and 302 and the operator's forceps 305 and 306 as the evaluation region, and sets the evaluation mesh 310 in the whole of the evaluation region. The operator's forceps 305 or 306 may be an energy treatment tool. Subsequently, the image processing system 100 deforms the evaluation mesh 310 following the tissue, and excludes a region that does not require analysis of deformation from the evaluation region. This narrows down a presentation range of the evaluation mesh 310.

With this configuration, it is possible to utilize dynamic information associated with the pulling and estimate a region in which the deformation of the tissue should be evaluated with high accuracy. As a result, it is possible to efficiently provide a region necessary for the user with the pulling information.

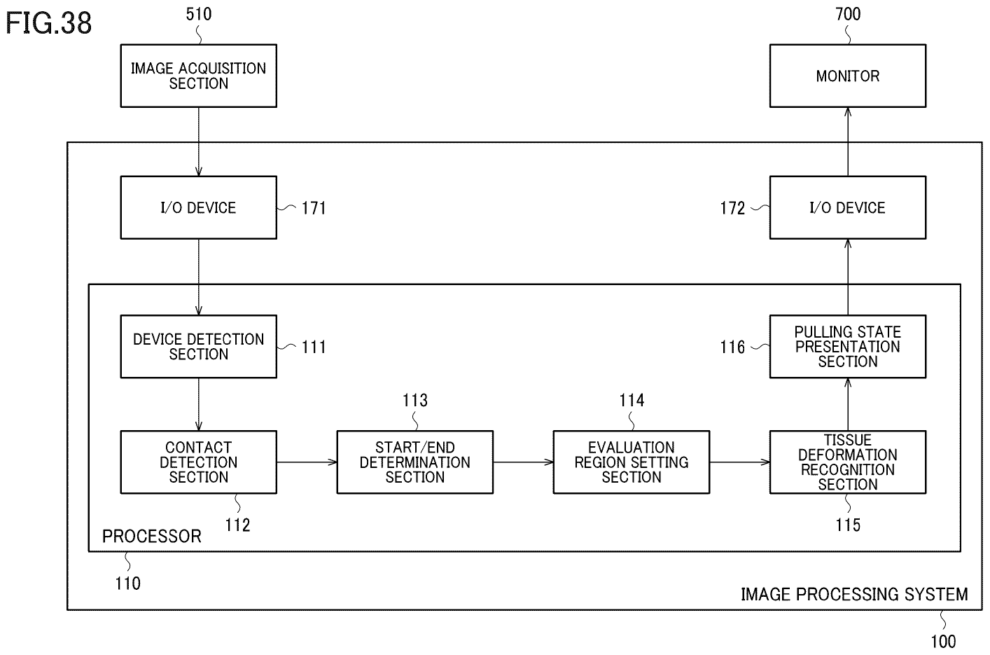

FIG. 38 illustrates a configuration example of a medical system in the second embodiment. The medical system includes an image acquisition section 510, the image processing system 100, and the monitor 700.

The image acquisition section 510 acquires endoscope images. The image acquisition section 510 corresponds to, for example, the endoscope 500 in FIG. 4. Alternatively, the image acquisition section 510 may read out the endoscope images from a storage in which the endoscope images are recorded.

The image processing system 100 includes an input/output (I/O) device 171, an I/O device 172, and the processor 110. Although not illustrated, the image processing system 100 may include the memory 120 and/or the operation section 150 similarly to FIG. 6.

The I/O device 171 receives image data of endoscope images from the image acquisition section 510, and inputs the image data of the endoscope images to the processor 110. The I/O device 172 transmits presentation information output from the processor 110 to the monitor 700. The I/O device 171 and the I/O device 172 correspond to the communication section 160 in FIG. 6.

The processor 110 includes a device detection section 111, a contact detection section 112, a start/end determination section 113, an evaluation region setting section 114, a tissue deformation recognition section 115, and a pulling state presentation section 116. Note that a correspondence relation with the first embodiment is as follows. Processing performed by the device detection section 111, the contact detection section 112, and the start/end determination section 113 corresponds to the pulling scene determining step S21 in FIG. 7. Processing performed by the evaluation region setting section 114 is newly added processing. Processing performed by the tissue deformation recognition section 115 corresponds to the pulling range estimating step S23 in FIG. 7. Additionally, processing performed by the pulling state presentation section 116 corresponds to the presentation information processing step S25 in FIG. 7.

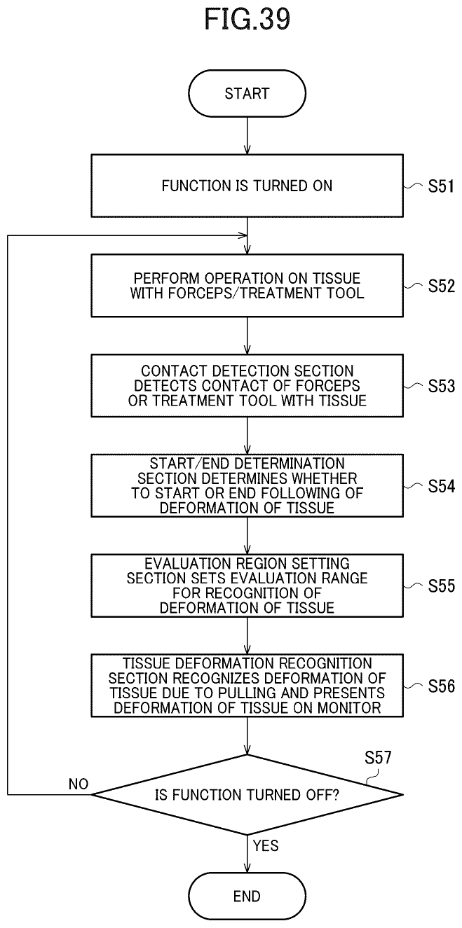

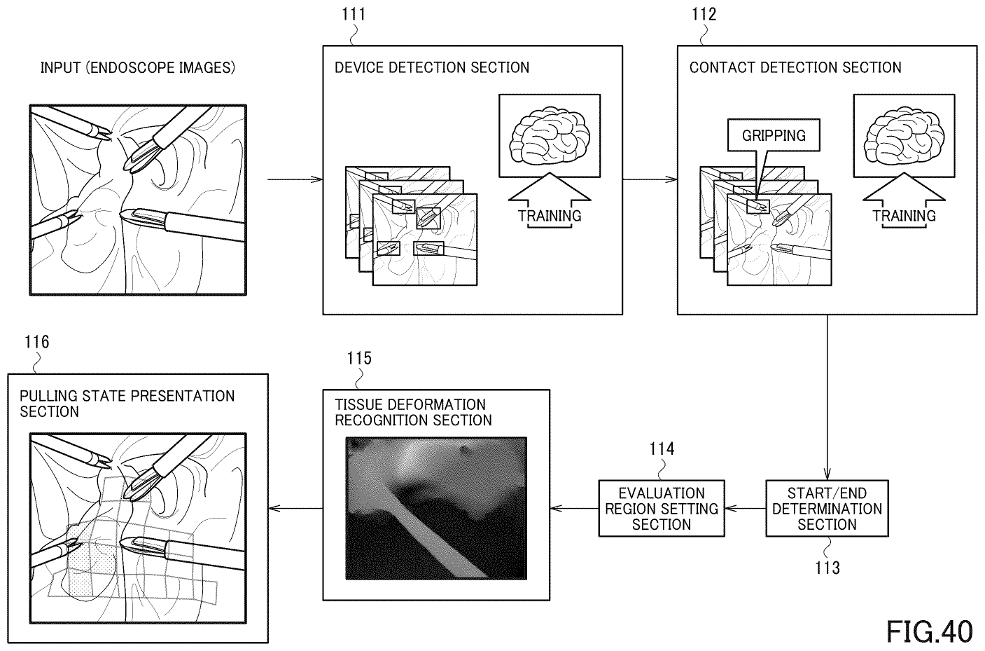

The description will be given below of processing performed by each section of the processor 110 with reference to FIGS. 39 and 40. Except for processing performed by the newly added evaluation region setting section 114, the description will be given as a mere example of processing, and the methods described in the pulling scene determining step S21, the pulling range estimating step S23, and the presentation information processing step S25 in the first embodiment may be adopted as appropriate.

As illustrated in FIG. 39, in step S51, a function of presenting the pulling state is turned ON. For example, the function may be turned ON by the user's operation, may be turned ON by scene recognition such as image recognition of a specific manipulation scene, or may be turned ON in response to an action on a device such as power-ON of the energy treatment tool.

In step S52, the user such as the operator and the assistant performs an operation on the tissue with the forceps or the treatment tool. The device detection section 111 detects the forceps or the treatment tool from the endoscope images. As illustrated in FIG. 40, the endoscope images are input to the device detection section 111, and the device detection section 111 uses a machine learning model to detect the forceps or the treatment tool from the endoscope images. The machine learning model is, for example, a model using object detection or a segmentation method. In a training phase, the machine learning model is subjected to machine learning so as to recognize the treatment tool or a jaw of the forceps from the endoscope images with endoscope images to which an annotation of the treatment tool or the jaw of the forceps is added as training data. The annotation is a boundary box that surrounds the treatment tool or the jaw of the forceps in the object detection, and is data that fills a region of the treatment tool or the jaw of the forceps with a color in segmentation. Note that the device detection section 111 may recognize the treatment tool or the jaw of the forceps from the endoscope images based on a detection algorithm based on a rule.

In step S53, the contact detection section 112 detects a contact state between the forceps or the treatment tool and the tissue from the endoscope images. As illustrated in FIG. 40, the contact detection section 112 uses the machine learning model to detect contact between the forceps or the treatment tool and the tissue from the endoscope images. In a training phase, the machine learning model is subjected to machine learning so as to recognize presence/absence of contact between the forceps or the treatment tool and the tissue from the endoscope images with endoscope images to which an annotation of the presence/absence of contact between the forceps or the treatment tool and the tissue is added as training data. Note that the contact detection section 112 may recognize the presence/absence of contact between the forceps or the treatment tool and the tissue from the endoscope images based on the detection algorithm based on the rule.

In step S54, the start/end determination section 113 determines whether to start or end processing of causing the evaluation mesh to follow the deformation of the tissue based on the contact state between the forceps or the treatment tool and the tissue which has been detected by the contact detection section 112, or a preliminarily set rule.

In step S55, the evaluation region setting section 114 sets the evaluation region for recognition of deformation of the tissue based on the endoscope images. That is, the evaluation region setting section 114 narrows down the evaluation region as necessary based on the endoscope images.

In step S56, the tissue deformation recognition section 115 recognizes the deformation of the tissue associated with the pulling or the like from the endoscope images. The pulling state presentation section 116 presents a result of the recognition by the tissue deformation recognition section 115 to the operator or the assistant. An image illustrated within a frame of the tissue deformation recognition section 115 in FIG. 40 represents the magnitude of a flow vector at each point detected by Optical Flow with brightness, and represents the direction of the flow vector with a hue. The tissue deformation recognition section 115 uses a result of detection from such Optical Flow to cause the evaluation mesh 310 to follow the object and thereby recognizes the deformation of the tissue. The pulling state presentation section 116 adds a color or the like depending on the deformation quantity in each cell in the evaluation mesh 310 to the endoscope images, and thereby presents the pulling state to the operator or the assistant.

In step S57, in a case where the function is turned OFF, the processor 110 ends the processing. In a case where the function is not turned OFF, the processing returns to step S52.

Note that the evaluation region that has been narrowed down in step S55 may be applied to the analysis of the deformation in step S56a. That is, the analysis of the deformation may be performed on the evaluation mesh 310 in the narrowed evaluation region. Alternatively, the evaluation region that has been narrowed down in step S55 may be applied to information presentation in step S56b. That is, in step S56a, the analysis of deformation may be performed on the evaluation mesh 310 in the evaluation region that has not been narrowed down, and in step S56b, the information presentation may be performed based on the deformation of the evaluation mesh 310 within the evaluation region that has been narrowed down in step S55 out of the evaluation mesh 310 on which the analysis of deformation has been performed.

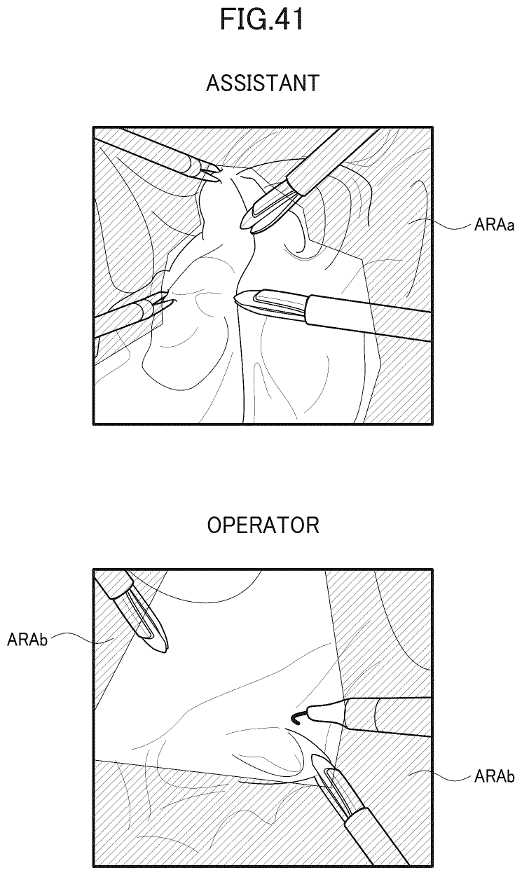

Methods of narrowing down the evaluation region (1-1) to (1-5) in step S55 are now described. First, an overview of each method is described with reference to FIGS. 41 to 45.

(1-1) Exclusion of a Range (Background) without Movement

FIG. 41 illustrates an example. The evaluation region setting section 114 excludes regions ARAa and ARAb in which the deformation quantity of each analysis point is a threshold or less for a predetermined period of time since the start of the function from the evaluation region. The deformation quantity of each analysis point is, for example, the magnitude of the movement quantity. The evaluation region setting section 114 may perform comparison processing between frames to correct at least one of shift, rotation, or scaling of the endoscope. For example, a correction method using a transformation matrix, which will be described later in a third embodiment, may be adopted. In the example in FIG. 41, the region ARAa to be excluded in processing for the assistant and the region ARAb to be excluded in processing for the operator are different. For example, the region ARAa to be excluded for the assistant is a region that is not moved by the pulling of the tissue with the assistant's forceps, and the region ARAb to be excluded for the operator is a region that is not moved by the pulling of the tissue with the operator's forceps. Note that a common evaluation region may be set without distinguishing between the processing for the operator and the processing for the assistant.

### (1-2) Narrowing Using Segmentation

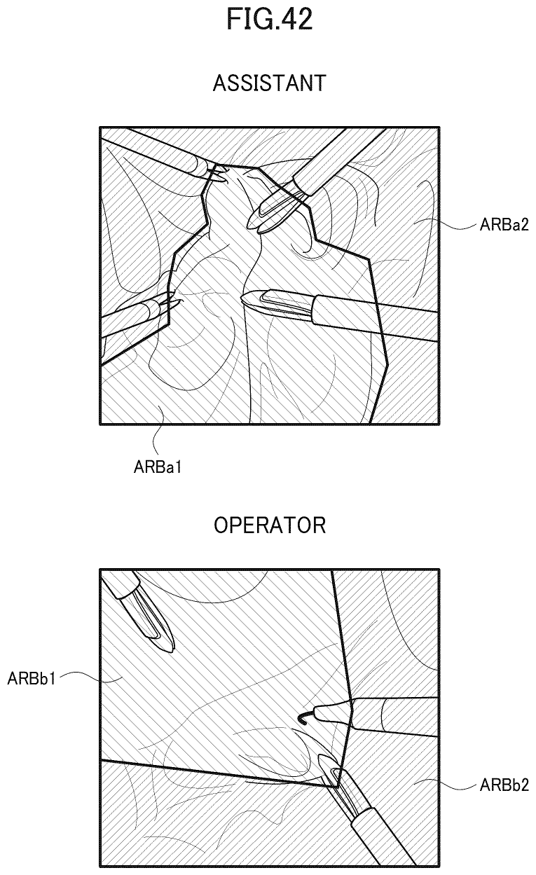

FIG. 42 illustrates an example. The evaluation region setting section 114 divides a region of an endoscope image by the segmentation method, and selects the evaluation region from a plurality of regions obtained by the division. The selection may be based on a rule, or may be implemented by use of a machine learning model. In the processing for the assistant, the endoscope image is divided into a region ARBa1 and a region ARBa2 by segmentation, and, for example, the region ARBa1 is set as the evaluation region. In the processing for the operator, the endoscope image is divided into a region ARBb1 and a region ARBb2 by segmentation, and, for example, the region ARBb1 is set as the evaluation region. Note that a common evaluation region may be set without distinguishing between the processing for the operator and the processing for the assistant.

### (1-3) Narrowing Using Detection of a Contour

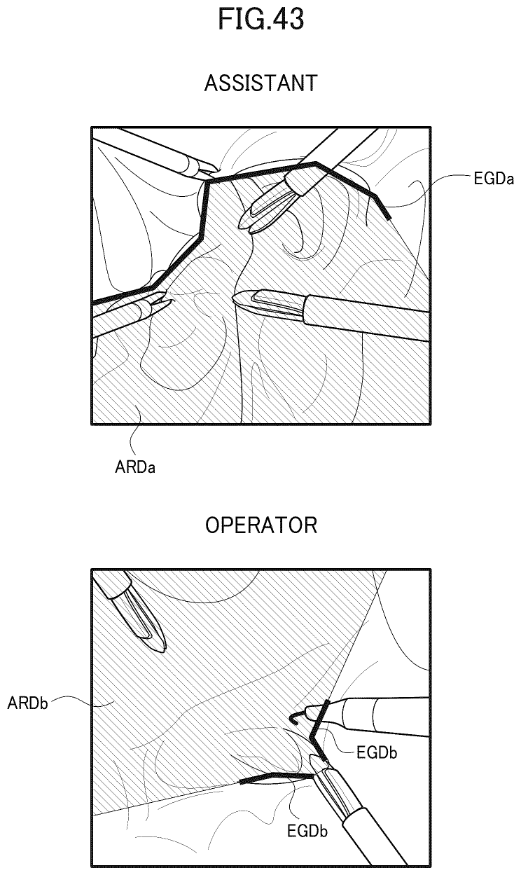

FIG. 43 illustrates an example. The evaluation region setting section 114 performs image processing to detect a contour (edge) of the tissue generated by the pulling. The detection of the contour is performed by, for example, Gabor filtering, Hough transformation, or the like. The evaluation region setting section 114 limits the evaluation region to a region closed with the detected contour and its extension line. In the processing for the assistant, a contour EGDa is detected from the endoscope image, and a region ARDa closed with the contour EGDa and its extension line is set as the evaluation region. In the processing for the operator, a contour EGDb is detected from the endoscope image, and a region ARDb closed with the contour EGDb and its extension line is set as the evaluation region. For example, the contour EGDa for the assistant is a contour of the tissue, which is generated by the pulling with the assistant's forceps, and the contour EGDb for the operator is a contour of the tissue, which is generated by the pulling with the operator's forceps. Note that a common evaluation region may be set without distinguishing between the processing for the operator and the processing for the assistant.

### (1-4) Narrowing Using Depth Information

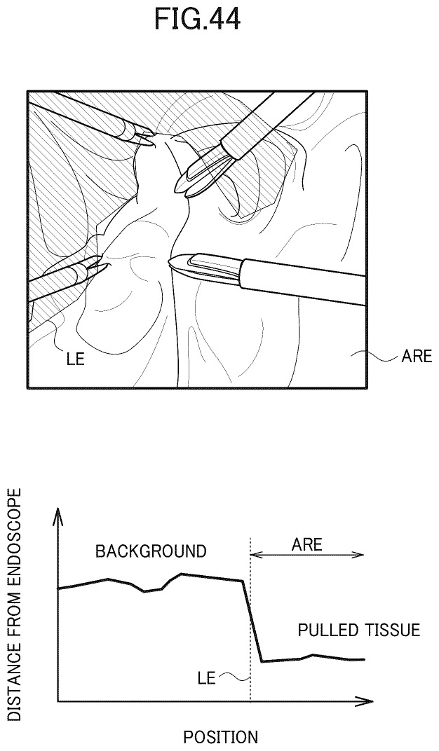

FIG. 44 illustrates an example. Especially, in a development scene using the assistant's forceps, a three-dimensional depth is different between the tissue to be pulled and the background. The depth mentioned herein is a distance from the endoscope to the tissue. The evaluation region setting section 114 narrows the evaluation region down to a region ARE on the front side in a depth direction with a line LE as a borderline on which the depth significantly changes. A lower chart in FIG. 44 illustrates a relationship between a position on an image and a distance (depth) from the endoscope. In this chart, the position is a one-dimensional position, but two-dimensional depth information is acquired in reality. The region ARE (on the front side in the depth direction) whose distance from the endoscope is small is set as the evaluation region with the line LE as the borderline on which the depth significantly changes. Examples of a means for acquiring three-dimensional information include a method of using a 3D endoscope utilizing a stereoscopic view or the like and a method of using a machine learning model to estimate a depth from a two-dimensional image.

### (1-5) Narrowing Using Detection in the Pulling Direction

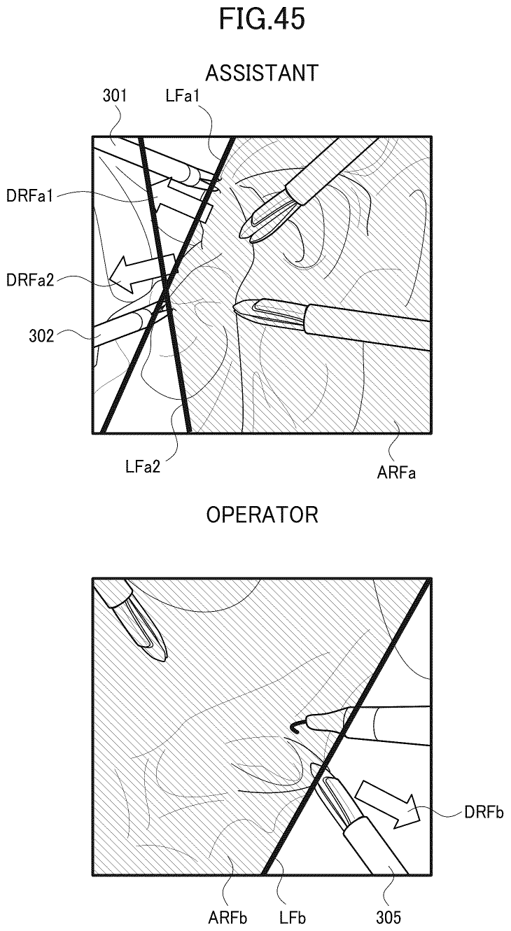

FIG. 45 illustrates an example. The evaluation region setting section 114 tracks the movement of the target forceps or the target treatment tool since the start of the function. The evaluation region setting section 114 narrows the evaluation region down to a region on the opposite side of the pulling direction with the gripping section of the forceps or the treatment tool as a criterion. Assume that the pulling direction mentioned herein is not a momentary movement direction such as between frames or the like, but is a movement direction from a criterion timing such as a time point at which the function starts. In the processing for the assistant, a region ARFa on the opposite side of a pulling direction DRFa1 with respect to a line LFa1 and on the opposite side of a pulling direction DRFa2 with respect to a line LFa2 is set as the evaluation region. The line LFa1 is a line that passes through the gripping section of the assistant's forceps 301 and that is orthogonal to the pulling direction DRFa1 of the assistant's forceps 301. The line LFa2 is a line that passes through the gripping section of the assistant's forceps 302 and that is orthogonal to the pulling direction DRFa2 of the assistant's forceps 302. In the processing for the operator, a region ARFb on the opposite side of a pulling direction DRFb with respect to a line LFb is set as the evaluation region. The line LFb is a line that passes through the gripping section of the operator's forceps 305 and that is orthogonal to the pulling direction DRFb of the operator's forceps 305. Note that a common evaluation region may be set without distinguishing between the processing for the operator and the processing for the assistant.

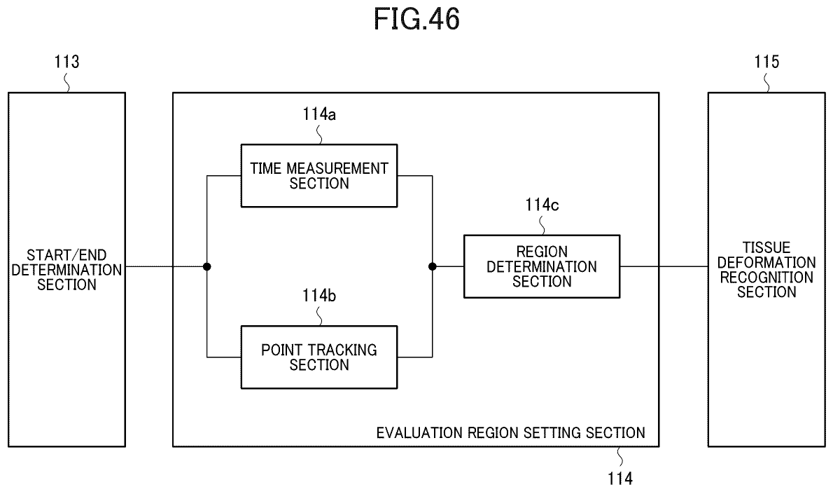

Subsequently, details of the above-mentioned methods (1-1) to (1-5) are now described with FIGS. 46 to 58.

(1-1a) First Detailed Example of Exclusion of the Range (Background) without Movement

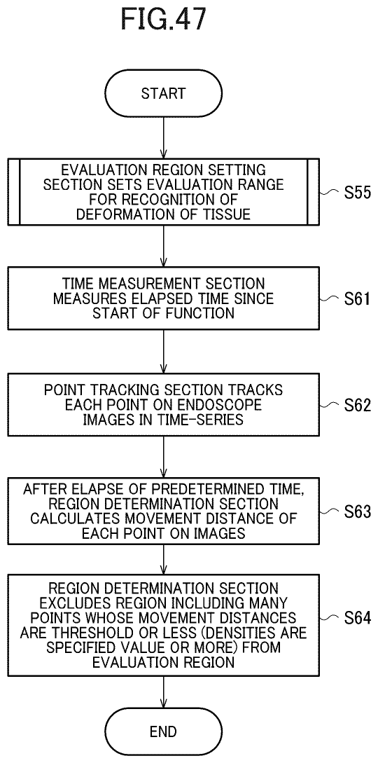

FIG. 46 illustrates a first detailed configuration example of the evaluation region setting section 114. The evaluation region setting section 114 includes a time measurement section 114a, a point tracking section 114b, and a region determination section 114c. FIG. 47 illustrates a flow of processing executed by the evaluation region setting section 114 in the first detailed configuration example. The flow is a detailed flow of step S55 in FIG. 39.

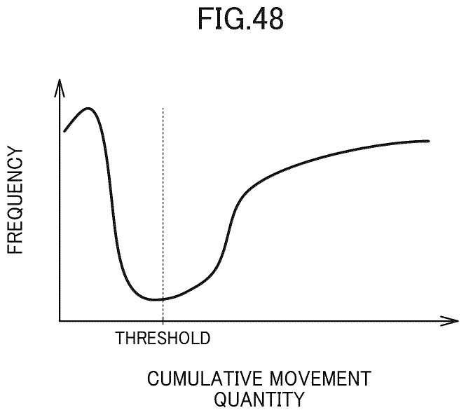

In step S61, the time measurement section 114a measures elapsed time since the criterion timing such as the start of the function. In step S62, the point tracking section 114b tracks each point on the endoscope images in time-series to cause each point to follow the movement of the object. In step S63, when the time measurement section 114a measures the elapse of certain time, the region determination section 114c calculates a cumulative movement quantity (cumulative movement distance) of each point on the endoscope images. The cumulative movement quantity is a quantity obtained by accumulating the movement quantity of each point at predetermined intervals such as between frames from the criterion timing such the start of the function. In step S64, the region determination section 114c excludes a region including many points whose cumulative movement quantities are a threshold or less from the evaluation region. The threshold is, for example, a fixed value that has been preliminarily determined, or a threshold, which will be described later with reference to FIG. 48. The region including may points whose cumulative movement quantities are the threshold or less is, for example, a region in which a density of points whose cumulative movement quantities are the threshold or less is a specified value or more.

Note that points to be tracked by the point tracking section 114b are points that are uniquely defined regardless of the evaluation mesh 310 and that are on the endoscope images. The points are, for example, set at higher density than analysis points of the evaluation mesh 310. Alternatively, the points to be tracked by the point tracking section 114b may be analysis points of the evaluation mesh 310 at this time point.

FIG. 48 illustrates an example of the threshold for the cumulative movement quantity. FIG. 48 is a so-called histogram, and represents a relationship between the cumulative movement quantity of each point and its frequency. The processor 110 determines the threshold based on this histogram. For example, the processor 110 determines a cumulative movement quantity that provides a minimum frequency value on the histogram as the threshold. Alternatively, the processor 110 may accumulate the frequency from one side of the histogram on which the cumulative movement quantity is small and determine the cumulative movement quantity when a cumulative value reaches a predetermined value as the threshold.

(1-1b) Second Detailed Example of Exclusion of the Range (Background) without Movement

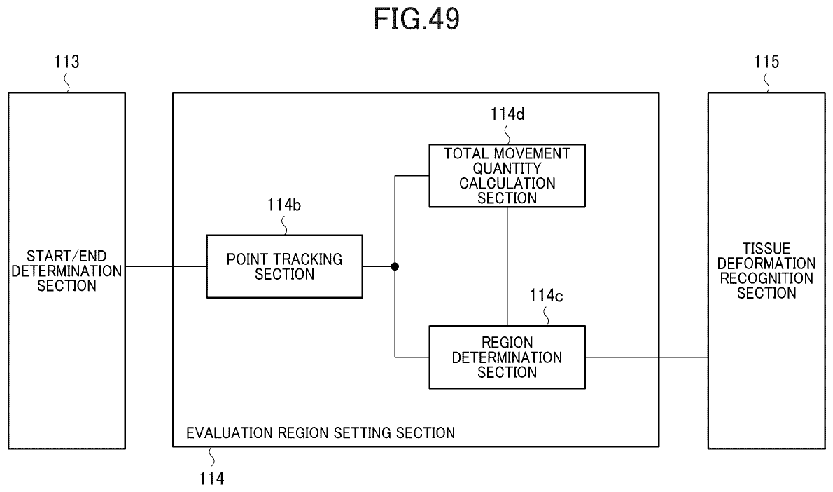

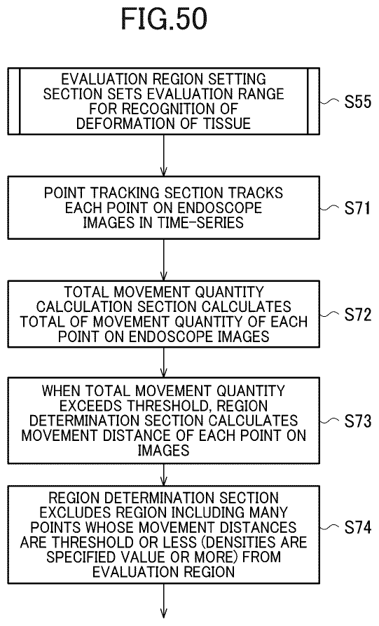

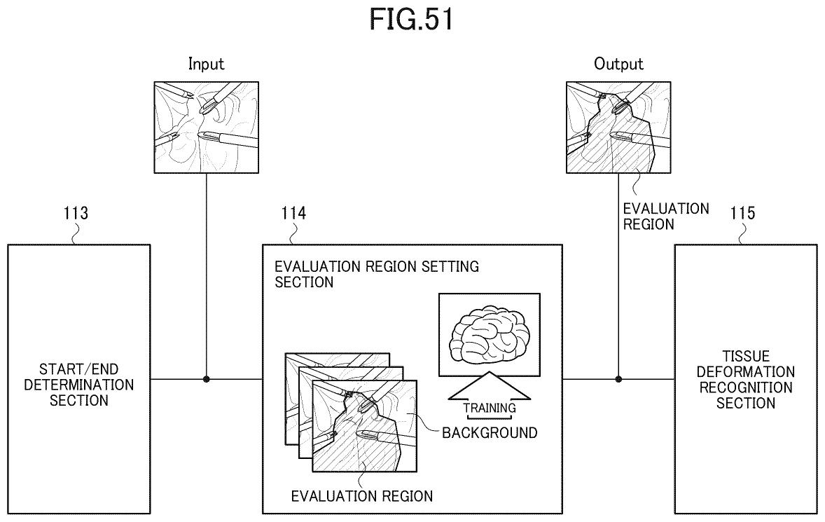

FIG. 49 illustrates a second detailed configuration example of the evaluation region setting section 114. The evaluation region setting section 114 includes the point tracking section 114b, a total movement quantity calculation section 114d, and the region determination section 114c. FIG. 50 illustrates a flow of processing executed by the evaluation region setting section 114 in the second detailed configuration example. The flow is a detailed flow of step S55 in FIG. 39.

In step S71, the point tracking section 114b tracks each point on the endoscope images in time-series to cause each point to follow the movement of the object. In step S72, the total movement quantity calculation section 114d aggregates the movement quantity of each point between frames or at predetermined intervals in the endoscope image, and accumulates a total value from the start of the function. Assume that the cumulative value is referred to as a total movement quantity. In step S73, when the total movement quantity exceeds a first threshold, the region determination section 114c calculates the cumulative movement quantity (cumulative movement distance) of each point on the endoscope images. The cumulative movement quantity is as defined above. The first threshold is, for example, a fixed value that has been preliminarily determined. In step S74, the region determination section 114c excludes a region including many points whose cumulative movement quantities are a second threshold or less from the evaluation region. The second threshold is similar to the threshold as described in (1-1a).

In accordance with the second detailed example, even in a case where the operator or the assistant temporarily stops the pulling after the start of the function, the exclusion of the evaluation region is not executed because the processing does not proceed to step S73 during the stop. When the pulling resumes, the processing proceeds to step S73 and the exclusion of the evaluation region is executed in step S74. With this configuration, it is possible to flexibly extend time until determination about the evaluation region is made and take measures.

### (1-2a) First Detailed Example of Narrowing Using Segmentation

FIG. 51 illustrates a third detailed configuration example of the evaluation region setting section 114. Endoscope images are input to the evaluation region setting section 114, and the evaluation region setting section 114 uses a segmentation model to estimate the evaluation region from the endoscope images. In a training phase, the segmentation model is subjected to machine learning so as to estimate the evaluation region from the endoscope images with endoscope images to which an annotation of the evaluation region is added as training data.

In accordance with to the present detailed example, it is possible to directly estimate the evaluation region as output from the segmentation model. Additionally, even in a scene including few movements due to the pulling, it is possible to estimate the evaluation region based on the images.

### (1-2b) Second Detailed Example of Narrowing Using Segmentation

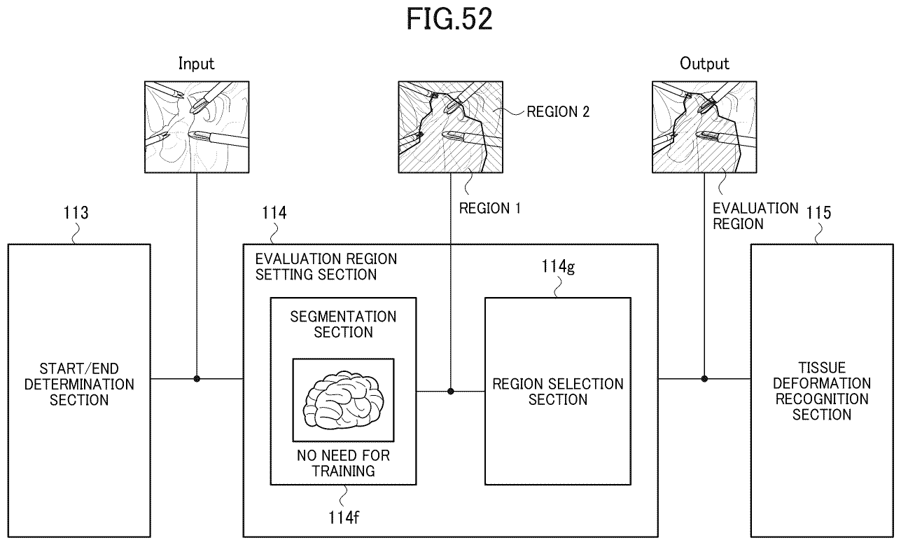

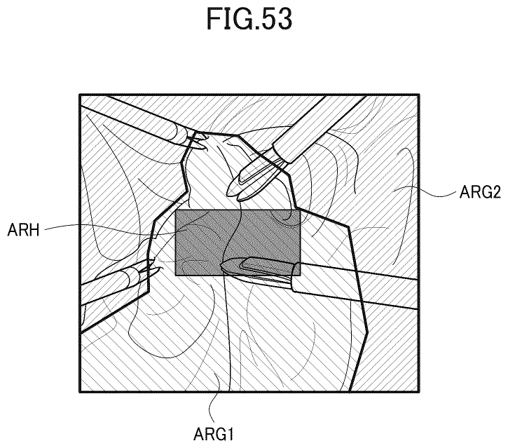

FIG. 52 illustrates a fourth detailed configuration example of the evaluation region setting section 114. The evaluation region setting section 114 divides a region using the segmentation method, and sets the evaluation region based on a rule. The evaluation region setting section 114 includes a segmentation section 114f and a region selection section 114g.

The segmentation section 114f divides an endoscope image into a plurality of regions using the segmentation method. FIG. 52 illustrates an example in which the endoscope image is divided into a region 1 and a region 2, but the number of regions is not limited to two. The segmentation method may be a non-AI method such as GrabCut, but may be an AI method such as Segment Anything Model (SAM). The AI mentioned herein is general-purpose AI, and does not require preliminary training. That is, training as the general-purpose AI has been performed, but special preliminary training on the assumption of application to the image processing system 100 in accordance with the present embodiment is not necessary. Additionally, the AI mentioned herein divides the image into regions, but does not estimate what each region is. That is, the AI does not determine whether or not the divided region is the evaluation region.

The region selection section 114g selects the evaluation region from the plurality of divided regions based on a rule. FIG. 53 illustrates an example. Assume that the segmentation section 114f divides the endoscope image into a region ARG1 and a region ARG2. The region selection section 114g selects a region having maximum overlap with a determination region ARH at the middle of the field of view as the evaluation region from the region ARG1 and the region ARG2. In the example in FIG. 53, since the region ARG1 has the maximum overlap with the determination region ARH, the region ARG1 is selected as the evaluation region. Alternatively, the region selection section 114g may select a region including one point at the middle of the field of view as the evaluation region from the region ARG1 and the region ARG2.

Preliminary training of the network on the assumption of application to the image processing system 100 in accordance with the present embodiment is necessary in (1-2a), while it is not necessary in (1-2b).

### (1-3) Detailed Example of Narrowing Using Detection of the Contour

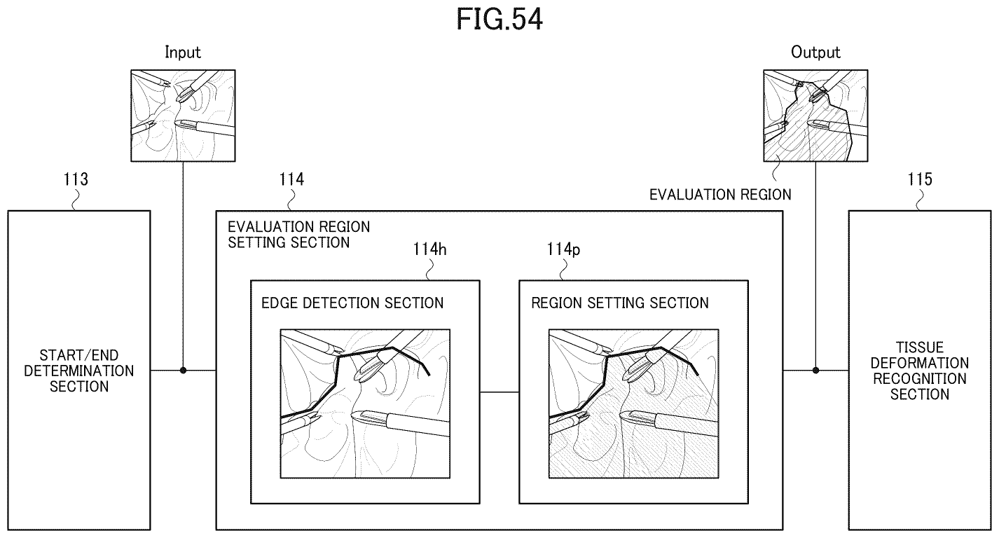

FIG. 54 illustrates a fifth detailed configuration example of the evaluation region setting section 114. The evaluation region setting section 114 includes an edge detection section 114h and a region setting section 114p.

The edge detection section 114h uses image processing to detect the contour (edge) of the tissue generated by the pulling from endoscope images. The detection of the contour is the non-AI method, and is performed by, for example, Gabor filtering, Hough transformation, or the like. The region setting section 114p extends the detected contour to generate a region closed with a contour line and an extension line, and limits the evaluation region to the region. The extension line is obtained by, for example, linearly extending an edge of the contour to an edge of the image. An example of the region closed with the contour line and the extension line is as illustrated in FIG. 43.

In accordance with the present detailed example, since the evaluation region is set using the non-AI algorithm, training of the network is not necessary. Additionally, since a calculation quantity is lower than that in the case of using the AI method, speed of processing is expected to increase or a calculation resource can be inhibited. With the inhibition of the calculation resource, implementation using a lower spec PC can be expected.

### (1-4) Detailed Example of Narrowing Using Depth Information

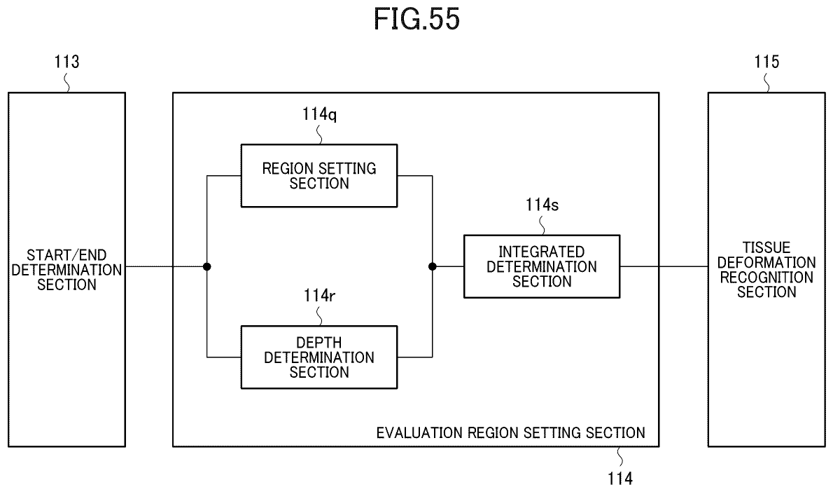

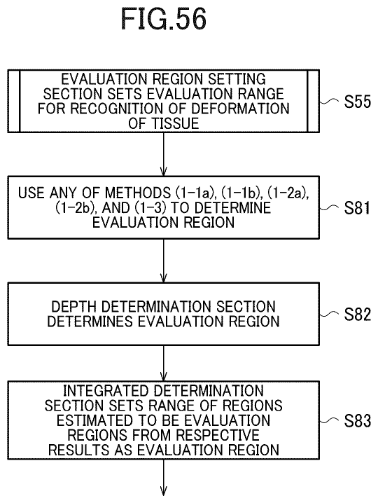

FIG. 55 illustrates a sixth detailed configuration example of the evaluation region setting section 114. The evaluation region setting section 114 adds depth information to the above-mentioned methods (1-1a), (1-1b), (1-2a), (1-2b), or (1-3) to determine the evaluation region. The evaluation region setting section 114 includes a region setting section 114q, a depth determination section 114r, and an integrated determination section 114s. FIG. 56 illustrates a flow of processing executed by the evaluation region setting section 114 in the sixth detailed configuration example. The flow is a detailed flow of step S55 in FIG. 39.

In step S81, the region setting section 114q uses any of the above-mentioned methods (1-1a), (1-1b), (1-2a), (1-2b), and (1-3) to determine the evaluation region. In step S82, the depth determination section 114r acquires depth information indicating distribution of a distance from the distal end of the endoscope to the target tissue, and determines a line on which the distance significantly changes in the depth information, from the threshold or the like. The depth determination section 114r divides regions with the line serving as a boundary, and determines a region on the front side in the depth direction, out of the divided regions, as the evaluation region. For example, the endoscope 500 in FIG. 5 is a 3D endoscope, and the depth determination section 114r uses depth information of a 3D image captured by the 3D endoscope. Alternatively, the endoscope 500 in FIG. 5 is a 2D endoscope, and the depth determination section 114r uses AI that estimates a depth value from a 2D image to estimate depth information from endoscope images. In step S83, the integrated determination section 114s sets a region that belongs to both of the evaluation region determined by the region setting section 114q and the evaluation region determined by the depth determination section 114r as the evaluation region.

Especially, in a scene where the assistant develops the tissue, a three-dimensional depth is different between the tissue to be pulled and the background. In accordance with the present detailed example, by combining the depth information, it is possible to set the evaluation region in consideration of the three-dimensional depth, thereby increasing accuracy of setting the evaluation region.

### (1-5) Detailed Example of Narrowing Using Detection in the Pulling Direction

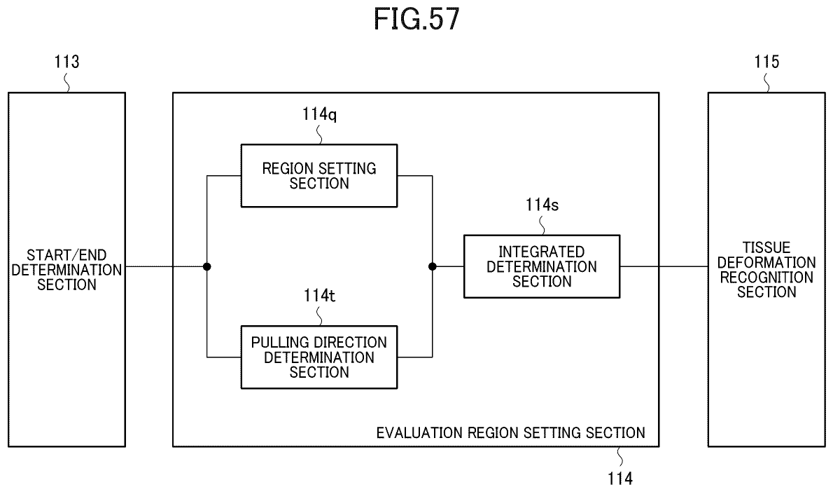

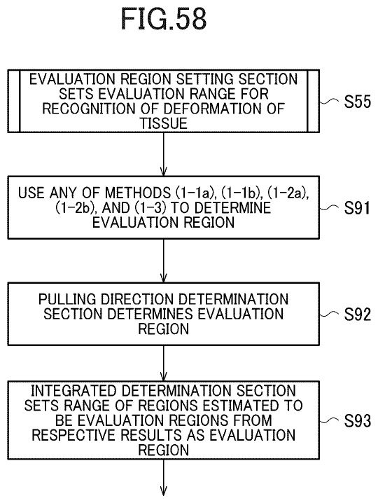

FIG. 57 illustrates a seventh detailed configuration example of the evaluation region setting section 114. The evaluation region setting section 114 adds information in the pulling direction to the above-mentioned method (1-1a), (1-1b), (1-2a), (1-2b), or (1-3) to determine the evaluation region. The evaluation region setting section 114 includes the region setting section 114q, a pulling direction determination section 114t, and the integrated determination section 114s. FIG. 58 illustrates a flow of processing executed by the evaluation region setting section 114 in the seventh detailed configuration example. The flow is a detailed flow of step S55 in FIG. 39.

In step S91, the region setting section 114q uses any of the above-mentioned methods (1-1a), (1-1b), (1-2a), (1-2b), and (1-3) to determine the evaluation region. In step S92, the pulling direction determination section 114t tracks the movement of the target forceps since the criterion timing such as the start of the function. The pulling direction determination section 114t, for example, detects the target forceps from the endoscope images, obtains a flow vector using Optical Flow from the endoscope images, and uses the flow vector at a position of the detected forceps to track the movement of the forceps. The pulling direction determination section 114t narrows the evaluation region down to an opposite side of the pulling direction with the gripping section of the forceps or the treatment tool as a criterion. This example is as illustrated in FIG. 45. In step S93, the integrated determination section 114s sets a region that belongs to both of the evaluation region determined by the region setting section 114q and the evaluation region determined by the pulling direction determination section 114t as the evaluation region.

The tissue on the pulling direction side with respect to the treatment tool is considered to be not a pulling target, and the tissue on the opposite side of the pulling direction with respect to the treatment tool is considered to be the pulling target. In accordance with the present detailed example, by combining the information regarding the pulling direction, it is possible to set the evaluation region in the tissue as the pulling target, thereby increasing accuracy of setting the evaluation region.

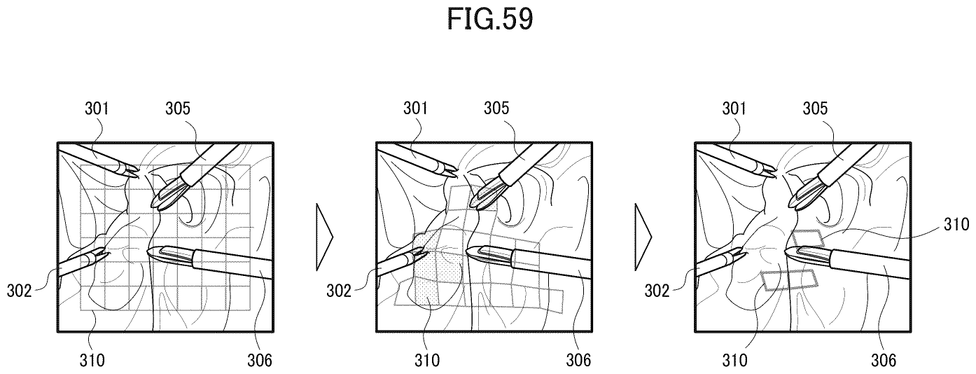

Step S55 in which the evaluation region is set in FIG. 39 is repeatedly executed while the function is ON. As a result, as illustrated in FIG. 59, the evaluation region in which the evaluation mesh 310 is set is gradually narrowed down over time. There is a possibility that the evaluation region is narrowed down too much such as in a case where the assistance function is used for long time, or in a case where the evaluation region is excessively narrowed down due to an error. Methods of addressing excessive narrowing, (2-1), (2-2), and (3) will be described below.

First, methods of inhibiting narrowing the region (2-1) and (2-2) are described.

### (2-1) Limited to Specified Time

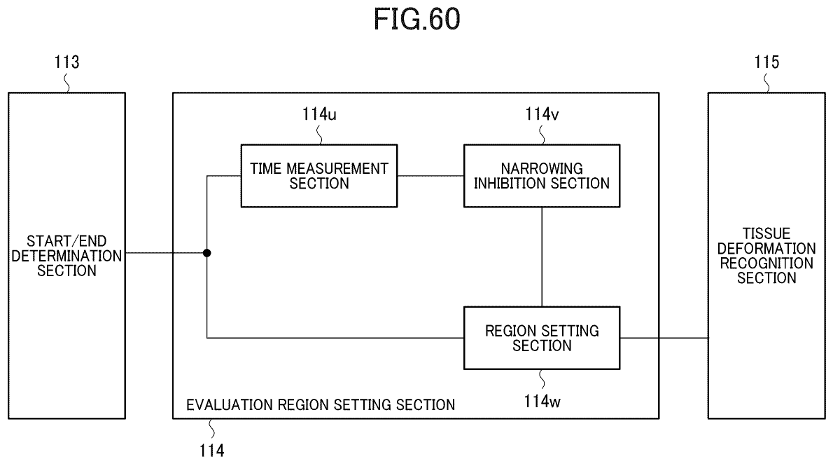

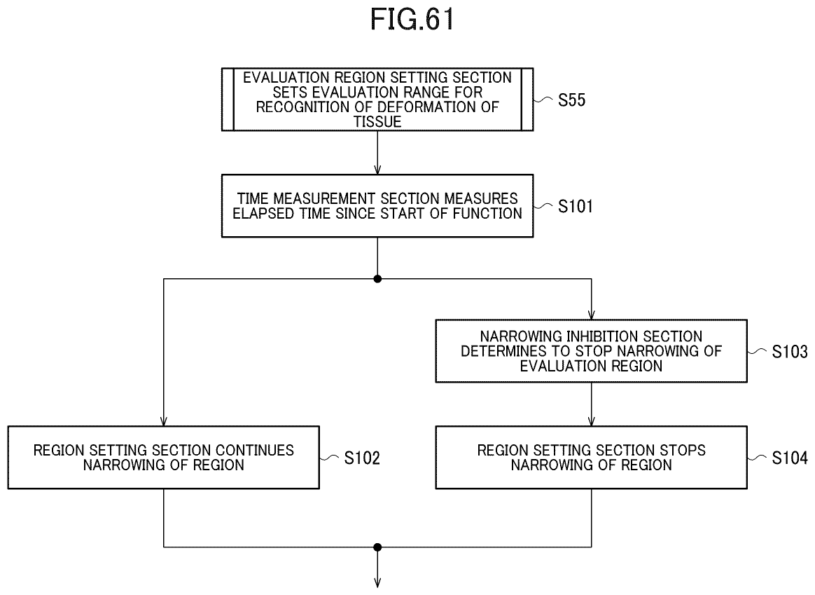

The evaluation region setting section 114 limits the execution of the narrowing of the evaluation region to only during specific time after the criterion timing such as the start of the function. FIG. 60 illustrates an eighth detailed configuration example of the evaluation region setting section 114. The evaluation region setting section 114 includes a time measurement section 114u, a narrowing inhibition section 114v, and a region setting section 114w. FIG. 61 illustrates a flow of processing executed by the evaluation region setting section 114 in the eighth detailed configuration example. The flow is a detailed flow of step S55 in FIG. 39.

In step S101, the time measurement section 114u measures elapsed time since the criterion timing such as the start of the function. In a case where the specified time has not elapsed, in step S102, the region setting section 114w continues narrowing of the evaluation region using any of the above-mentioned methods (1-1) to (1-5) or a method combining two or more of (1-1) to (1-5). In a case where the specified time has elapsed, in step S103, the narrowing inhibition section 114v determines to stop the narrowing of the evaluation region. In step S104, the region setting section 114w stops the narrowing of the evaluation region based on the determination about stop by the narrowing inhibition section 114v.

In accordance with the present method, since the narrowing of the evaluation region stops with the elapse of the specified time, it is possible to inhibit excessive narrowing of the evaluation region in comparison with a case where the narrowing continues.

### (2-2) Change the Threshold Over Time

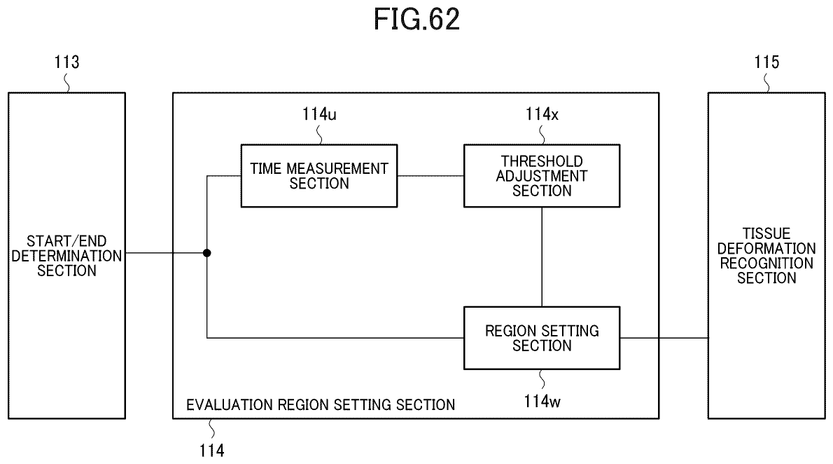

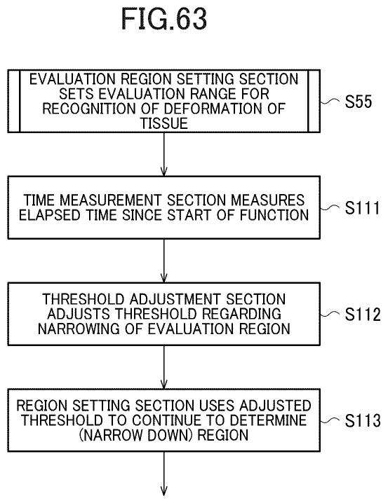

The evaluation region setting section 114 increases the threshold to be used for determination about the narrowing of the evaluation region with the elapse of time after the start of the function. The increased threshold makes it harder for the evaluation region to be narrowed down. FIG. 62 illustrates a ninth detailed configuration example of the evaluation region setting section 114. The evaluation region setting section 114 includes the time measurement section 114u, a threshold adjustment section 114x, and the region setting section 114w. FIG. 63 illustrates a flow of processing executed by the evaluation region setting section 114 in the ninth detailed configuration example. The flow is a detailed flow of step S55 in FIG. 39.

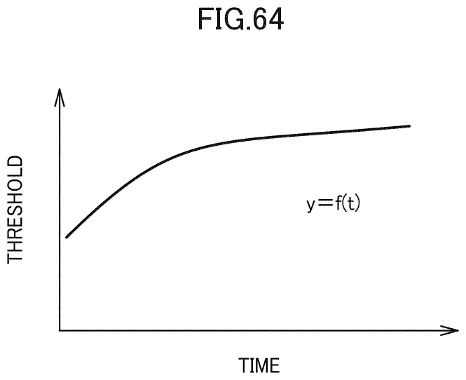

In step S111, the time measurement section 114u measures elapsed time since the criterion timing such as the start of the function. In step S112, the threshold adjustment section 114x adjusts a threshold regarding the narrowing of the evaluation region depending on elapsed time. In step S113, the region setting section 114w uses the adjusted threshold to continue the narrowing of the evaluation region. For example, the region setting section 114w uses the above-mentioned method (1-1) or (1-4) to narrow down the evaluation region. In a case of using the method (1-1), the threshold adjustment section 114x adjusts the threshold for the movement quantity. In a case of using the method (1-4), the threshold adjustment section 114x adjusts the threshold for the distance that defines the line on which the depth significantly changes. FIG. 64 illustrates an example of a change in threshold. The threshold adjustment section 114x adjusts the threshold so as to become a function y=f(t). The function is a function of elapsed time. The function y=f(t) is a function that causes the threshold to monotonically increase over time. However, this does not exclude a function including an interval in which the threshold does not increase, or a function including an interval in which the threshold temporarily decreases.

Subsequently, a method of extending the evaluation region that has been narrowed down once (3) is described.

### (3) Extension of the Evaluation Region

For example, in assistant for the assistant's pulling or the like, it is assumed to apply an assistant function to present the pulling state while the pulling is performed for a long period of time. In such a case, if the narrowing using any of the above-mentioned (1-1) to (1-5), there is a possibility that the evaluation region is narrowed down too much including an error in the narrowing. Additionally, after the start of the function, there is also a possibility that a new region that should be set as the evaluation region is seen due to reduction of the field of view.

Hence, the evaluation region setting section 114 newly determines the evaluation region using any of the above-mentioned methods (1-1) to (1-5) or a method combining two or more of the methods (1-1) to (1-5) every certain period of time since the start of the function. In a case where a difference between an evaluation region at this step and a newly determined evaluation region is large, that is, in a case where the evaluation region has been narrowed down too much, the evaluation region setting section 114 applies the newly determined evaluation region to extend the evaluation region. With this configuration, it is possible to extend the evaluation region that has been excessively narrowed down.

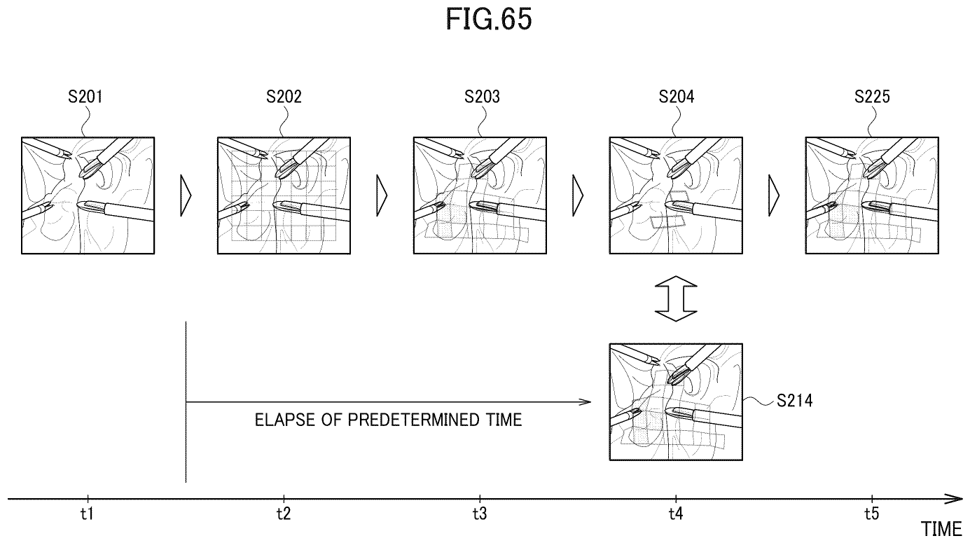

FIG. 65 illustrates an example. In step S201 at a time t1, the function is turned ON. In steps S202 to S204 at times t2 to t4, respectively, the evaluation region setting section 114 continues to determine the evaluation region since the start of the function. In step S203 at the time t3 and step S204 at the time t4, the evaluation region is gradually narrowed down. Aside from steps S202 to S204, the evaluation region setting section 114 newly determines the evaluation region in step S214 at the time t4 after the elapse of a certain period of time since the start of the function. The evaluation region setting section 114 compares the evaluation region in step S204 and the evaluation region in step S214. In a case where the evaluation region determined in step S214 is larger and a difference between the evaluation region determined in step S214 and the evaluation region determined in step S204 is large, the evaluation region setting section 114 adopts the evaluation region determined in step S214 in step S225 at a time t5. In a case where the condition is not satisfied, the evaluation region setting section 114 continues to use the evaluation region determined in step S204. Thereafter, the evaluation region setting section 114 updates the evaluation region using a method similar to the above-mentioned method every certain period of time.

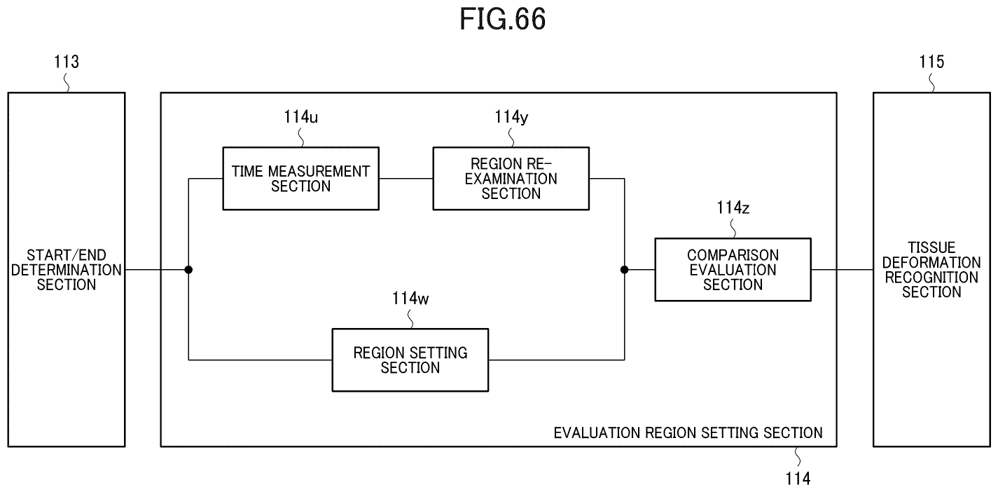

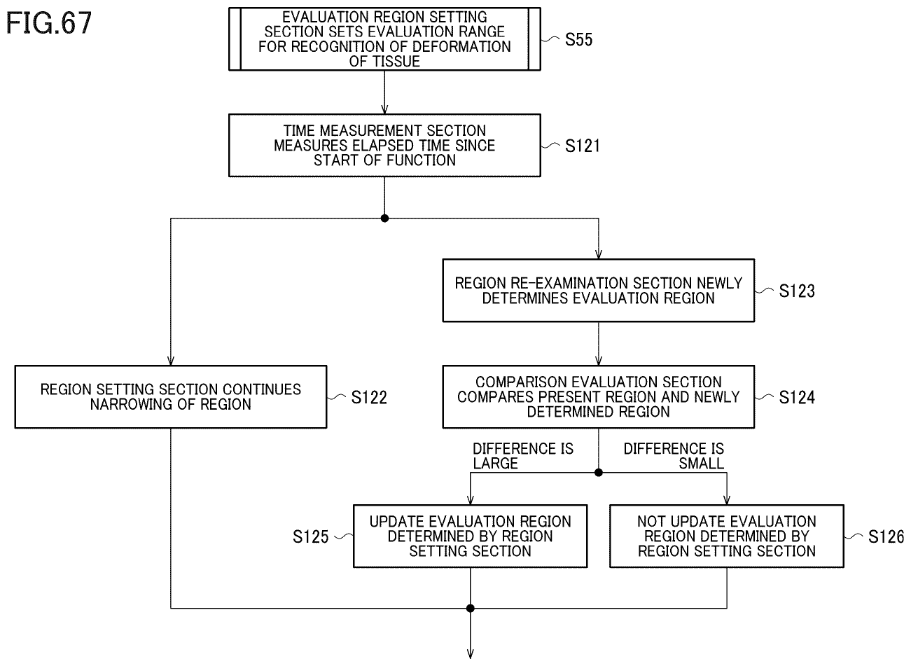

FIG. 66 illustrates a tenth detailed configuration example of the evaluation region setting section 114. The evaluation region setting section 114 includes the time measurement section 114u, a region re-examination section 114y, the region setting section 114w, and a comparison evaluation section 114z. FIG. 67 illustrates a flow of processing executed by the evaluation region setting section 114 in a tenth detailed configuration example. The flow is a detailed flow of step S55 in FIG. 39.

In step S121, the time measurement section 114u measures elapsed time since the criterion timing such as the start of the function. In step S122, the region setting section 114w continues narrowing of the evaluation region using any of the above-mentioned methods (1-1) to (1-5) or a method combining two or more of (1-1) to (1-5) regardless of the elapsed time. In step S123, the region re-examination section 114y newly determines the evaluation region using any of the above-mentioned methods (1-1) to (1-5) or a method combining two or more of (1-1) to (1-5) every certain period of time. In step S124, the comparison evaluation section 114z compares the present evaluation region determined by the region setting section 114w and the new evaluation region determined by the region re-examination section 114y. In the example in FIG. 65, this step corresponds to the comparison made in steps S204 and S214. In a case where a difference between the present evaluation region and the new evaluation region is large, the comparison evaluation section 114z updates the evaluation region determined by the region setting section 114w with the new evaluation region. In a case where the difference between the present evaluation region and the new evaluation region is small, the comparison evaluation section 114z does not update the evaluation region determined by the region setting section 114w. As a specific example, the comparison evaluation section 114z compares the present evaluation region determined by the region setting section 114w and the new evaluation region determined by the region re-examination section 114y based on areas. In a case where a ratio of the area of the present evaluation region to the area of the new evaluation region is a specified value or less, the comparison evaluation section 114z updates the evaluation region held by the region setting section 114w with the new evaluation region. The threshold is, for example, 20%, but is not limited thereto.

Other examples of the method of setting the evaluation region (4) and (5) are now described.

### (4) Manually Setting the Evaluation Region (Fixed)

The user sets the evaluation region before surgery or during surgery. The user may be either the operator or the assistant. For example, a plurality of options for the evaluation region may be provided and the user may select the evaluation region from the options. Conceivable examples of an operation section for making selection during surgery include a button on the scope, a button on the treatment tool, a foot switch, and an audio operation. The evaluation region setting section 114 sets the evaluation region input by the user's operation. In accordance with the present method, the user can utilize the function depending on his/her preference at a necessary timing.

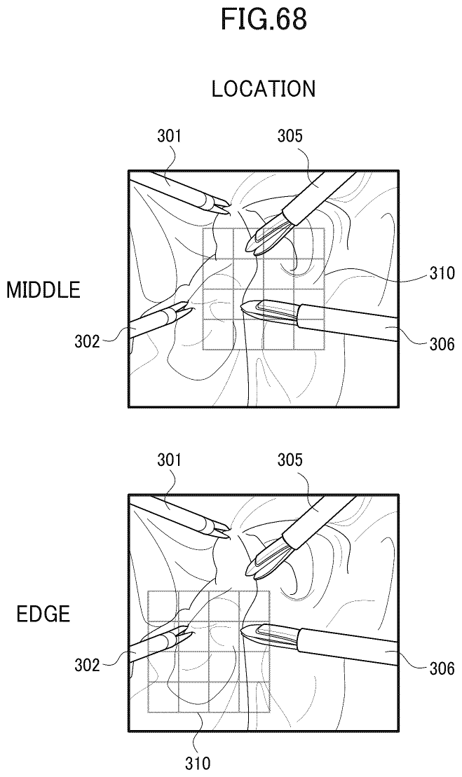

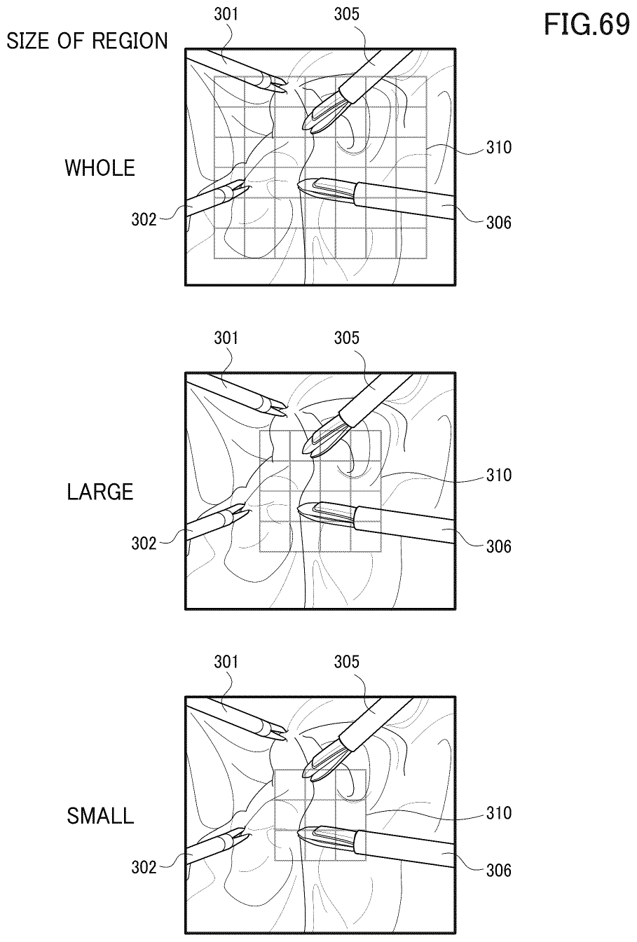

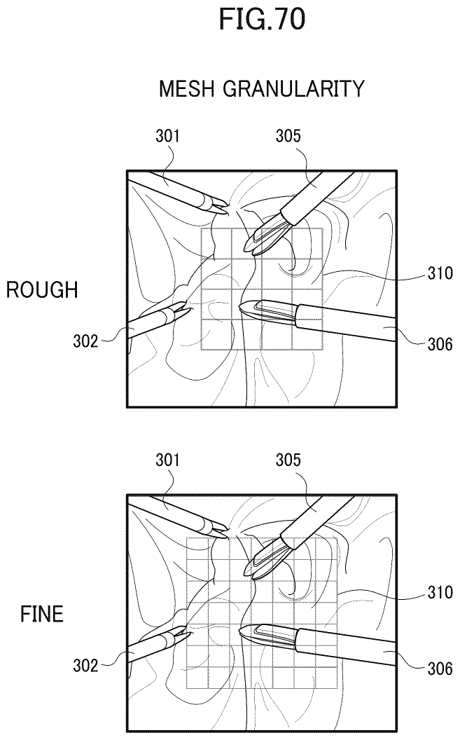

FIGS. 68 to 70 each illustrate an example of options for the evaluation region in which the evaluation mesh 310 is disposed. As illustrated in FIG. 68, the options may be options for a location of the evaluation region in the image. In the example in FIG. 68, a middle of the image and an edge of the image are options for the location. As illustrated in FIG. 69, the options may be options for a size of the evaluation region. In the example in FIG. 69, the whole of the image, a large size, and a small size are the options for the size. As illustrated in FIG. 70, the options may be granularity of the evaluation mesh 310. In the example in FIG. 70, crude and fine are the options for the granularity.

### (5) Setting of the Evaluation Region in the Vicinity of the Operator's Forceps

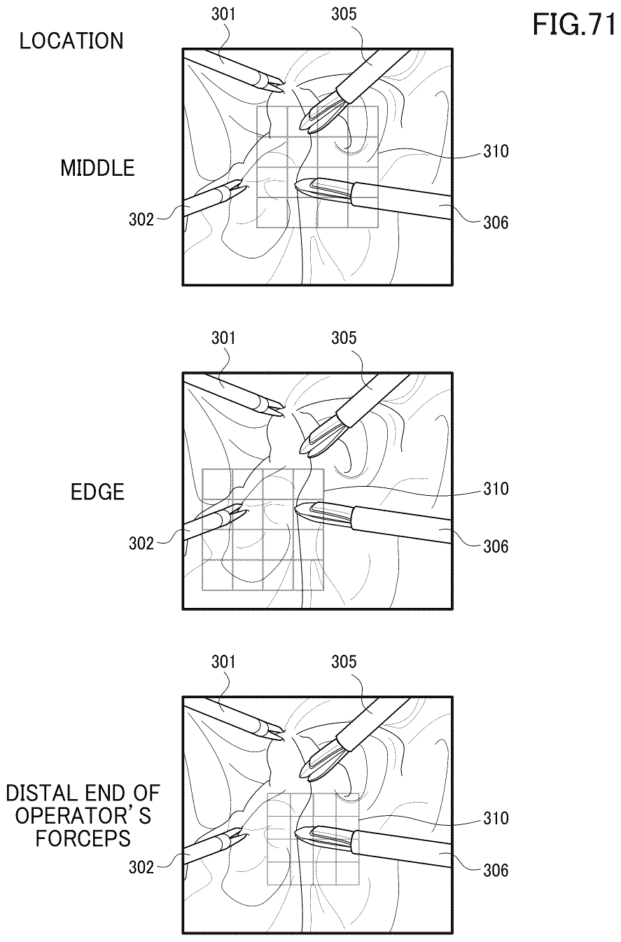

The evaluation region setting section 114 sets the evaluation region in the vicinity of the distal end of the forceps operated by the user. As illustrated in a lower view in FIG. 71, in a case where the user is the operator, the evaluation region is set in the vicinity of the distal end of the operator's forceps 306. As illustrated in FIG. 71, the evaluation region setting section 114 may add the vicinity of the distal end of the operator's forceps as one of the above-mentioned options for the location of the evaluation region.

The evaluation region setting section 114 utilizes a preliminarily trained network to detect a distal end portion of the operator's forceps from the endoscope images, and sets the evaluation region having a specified size in the distal end portion. For example, the device detection section 111 in FIG. 40 uses a machine learning model to detect the treatment tool or the jaw of the forceps from the endoscope images. The evaluation region setting section 114 sets the evaluation region at a position of the detected treatment tool or the detected jaw of the forceps.

In the present embodiment, the processor 110 may determine the evaluation region, which is a region in which the evaluation mesh 310 is disposed or a region to be reflected on an image display out of the evaluation mesh 310, based on at least one of a movement quantity of an object between the time-series images, an image characteristic quantity of the object, or depth information of the object. Note that the determination of the evaluation region based on the image characteristic quantity of the object corresponds to, for example, the narrowing using segmentation in FIG. 42, the narrowing using the detection of the contour in FIG. 43, the narrowing using the detection in the pulling direction in FIG. 45, or the like.

In accordance with the present embodiment, it is possible to narrow down to a tissue region as the treatment target or a tissue region from which the user needs the pulling information to present the pulling information. Additionally, the evaluation region is narrowed down, whereby the analysis of deformation of the evaluation mesh 310 becomes less susceptible to a tissue region that is not the treatment target, or a tissue region from which the user does not need the pulling information.

Additionally, in the present embodiment, the processor 110 may calculate, at each point of the object in the time-series images, a cumulative movement quantity obtained by accumulation of the movement quantity between frames or at predetermined intervals since the criterion timing until elapse of a predetermined period of time, and exclude a region in which the cumulative movement quantity is a threshold or less to set the evaluation region.

A region in which the movement quantity is low is considered to be a tissue region in which the treatment is not being performed such as a background region. In accordance with the present embodiment, by excluding the region in which the movement quantity is the threshold or less in a predetermined period of time and setting the evaluation region, it is possible to narrow down to the tissue region as the treatment target and set the evaluation region.

Additionally, in the present embodiment, the processor 110 may calculate the movement quantity of each point of the object in the time-series images, aggregate the movement quantity of each point in the image to calculate a total value, and accumulate the total value since the criterion timing to calculate a total movement quantity. When the total movement quantity exceeds a first threshold, the processor 110 may calculate the cumulative movement quantity obtained by accumulation of the movement quantity of each point between frames or at predetermined intervals since the criterion timing until the total movement quantity exceeds the first threshold, and exclude a region in which the cumulative movement quantity is a second threshold or less to set the evaluation region.

Since there is little movement in the endoscope images as a whole when the user does not perform the pulling, there is a possibility that the evaluation region is not set accurately if a region with little movement is excluded. In accordance with the present embodiment, when the total value of the movement quantity in the images becomes a certain value or more, that is, when it can be determined that the tissue in the images is moved by the pulling or the like, the determination about exclusion of the evaluation region is performed. With this configuration, the evaluation region is set in the tissue region that has been moved by the pulling or the like, and the region that has not been moved is excluded from the evaluation region, whereby the evaluation region is set accurately.

Additionally, in the present embodiment, the processor 110 may input the time-series images to a trained model that performs segmentation to estimate the evaluation region from the endoscope images, and set the evaluation region based on a result of the estimation by the trained model. Note that, for example, the trained model 122 in FIG. 6 includes a segmentation model and the evaluation region is estimated by the segmentation model.

According to the present embodiment, it is possible to directly estimate the evaluation region from the endoscope images by the segmentation. Even in a case where there is no information regarding the movement of the tissue or the like, it is possible to set the evaluation region.

Additionally, in the present embodiment, the processor 110 may input the time-series images to a trained model that performs segmentation to divide each image into regions, and set the evaluation region in a region having the largest overlap with a predetermined region in each image out of a plurality of regions divided by the trained model. Note that the predetermined region is, for example, the determination region ARH in FIG. 53, or one point at the middle of the field of view. Note that, for example, the trained model 122 in FIG. 6 includes a segmentation model and the time-series images are divided into regions by the segmentation model.

In accordance with the present embodiment, there is no need for training so as to enable estimation of the evaluation region from the endoscope images, which makes it possible to utilize, for example, an existing segmentation model that performs division into regions. This can reduce training cost. Also in the present embodiment, even in a case where there is no information regarding the movement of the tissue or the like, it is possible to set the evaluation region.

Additionally, in the present embodiment, the processor 110 may perform edge detection processing on the time-series images and set a closed region closed with a detected edge as the evaluation region. Note that, in a case where the closed region can be generated only with the edge in the image, the closed region closed with the edge is this region. Alternatively, as illustrated in FIG. 43, in a case where the closed region cannot be generated only with the edge, the closed region closed with the edge is a closed region closed with the edge and an extension line from the edge.

In accordance with the present embodiment, the evaluation region is set using the non-AI algorithm, training of the network is not necessary. Additionally, since a calculation quantity is lower than that in the case of using the AI method, speed of processing is expected to increase or a calculation resource can be inhibited. With the inhibition of the calculation resource, implementation using a lower spec PC can be expected.

Additionally, in the present embodiment, the processor 110 may detect the pulling direction of the treatment tool that pulls the object from the time-series images, and set the evaluation region on the opposite side of the pulling direction with respect to a predetermined position on the treatment tool. Note that, the “treatment tool” is only required to be a treatment tool capable of pulling the tissue, and may be, for example, forceps as a non-energy treatment tool, or an energy treatment tool having a jaw such as a bipolar device. Note that, in the example in FIG. 45, the predetermined position on the treatment tool is a position of the jaw of the forceps, but is not limited thereto, and may be a freely-selected position on the treatment tool.

A tissue on the pulling direction side with respect to the predetermined position on the treatment tool is considered to be a tissue, which is not the pulling target. A tissue on the opposite side thereof is considered to be a tissue as the pulling target. In accordance with the present embodiment, it is possible to set the evaluation region in the tissue as the pulling target, thereby increasing accuracy of setting the evaluation region.

Additionally, in the present embodiment, the processor 110 may acquire depth distribution from the endoscope to the object, and set the evaluation region in a region on a smaller depth side as a line on which the depth steeply changes in the depth distribution as a boundary. Note that, in the example in FIG. 44, the line LE corresponds to the line on which the depth steeply changes in the depth distribution, and the region ARE corresponds to the region on the smaller depth side.

For example, in a scene where the assistant develops the tissue or other scenes, there is a case where a three-dimensional depth is different between the tissue to be pulled and the background. In accordance with the present embodiment, it is possible to set the evaluation region in consideration of the three-dimensional depth, thereby increasing accuracy of setting the evaluation region.

Additionally, in the present embodiment, the processor 110 may determine to exclude part of the evaluation region or maintain the evaluation region based on at least one of the movement quantity, the image characteristic quantity, or the depth information at every given update timing to update the evaluation region. Note that in the example in FIG. 65, the time t4 or the time t5 corresponds to the given update timing.

In accordance with the present embodiment, it is possible to set the appropriate evaluation region depending on a situation that changes from moment to moment. Additionally, an unnecessary evaluation region is gradually excluded, whereby a display unnecessary for the user is reduced.

### 4. Third Embodiment

A configuration of the medical system is similar to the configuration of the medical system described with reference to FIG. 5 in the first embodiment. Additionally, a configuration of the image processing system 100 is similar to the configuration of the image processing system 100 described with reference to FIG. 6 in the first embodiment.

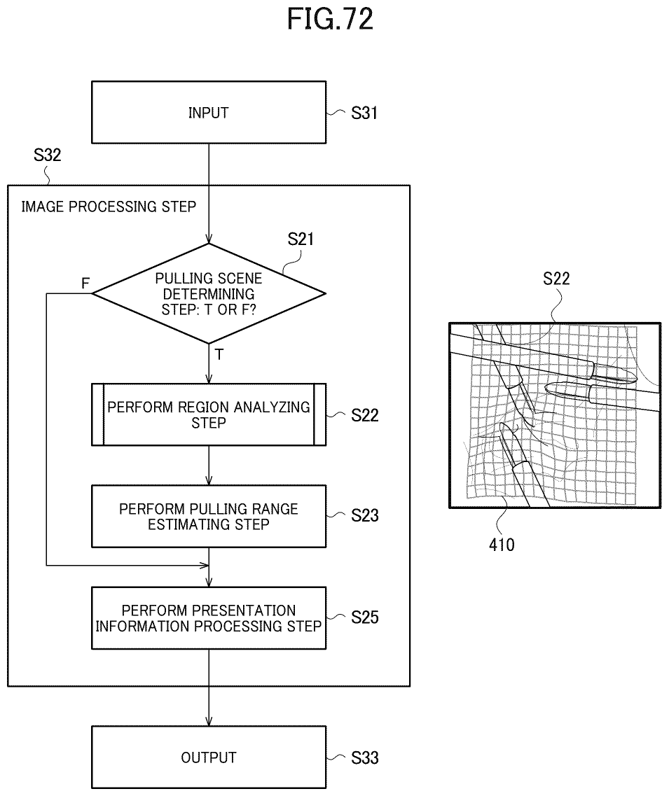

FIG. 72 illustrates a detailed flow of the image processing step S32 in a third embodiment. The pulling scene determining step S21, the pulling range estimating step S23, and the presentation information processing step S25 are similar to those described in FIG. 7 in the first embodiment. In these steps, various kinds of processing described with reference to FIGS. 8 to 35 can be adopted. The region analyzing step S22 is added in the third embodiment. That is, in a case of determining that a scene is the scene in which the pulling range should be estimated in step S21 (T: True), the processor 110 calculates the movement quantity of the tissue in the endoscope images in step S22, and analyzes the evaluation region based on a result of the calculation. In step S23, the processor 110 uses the analyzed evaluation region to estimate the pulling range.

Specifically, the processor 110 sets an evaluation mesh 410 at a time point at which the scene is the pulling scene in step S21, and sets the evaluation mesh 410 at this time point as an initial state. The processor 110 uses a result of calculation of the flow vector to moment in step S22 to analyze and update the evaluation mesh 410. The analyzed and updated evaluation mesh 410 is used for estimation of the pulling range in the pulling range estimating step S23 in a subsequent stage.

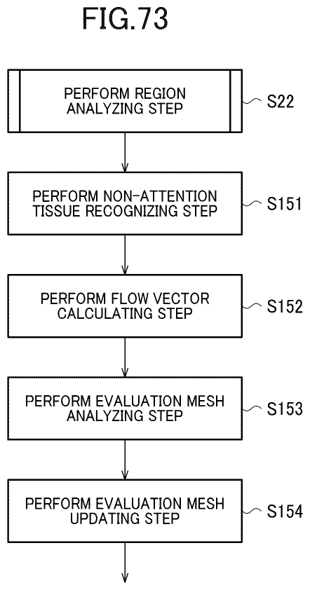

FIG. 73 illustrates a detailed flow of the region analyzing step S22. The region analyzing step S22 includes a non-attention tissue recognizing step S151, a flow vector calculating step S152, an evaluation mesh analyzing step S153, and an evaluation mesh updating step S154. Contents of processing in each step will be described below.

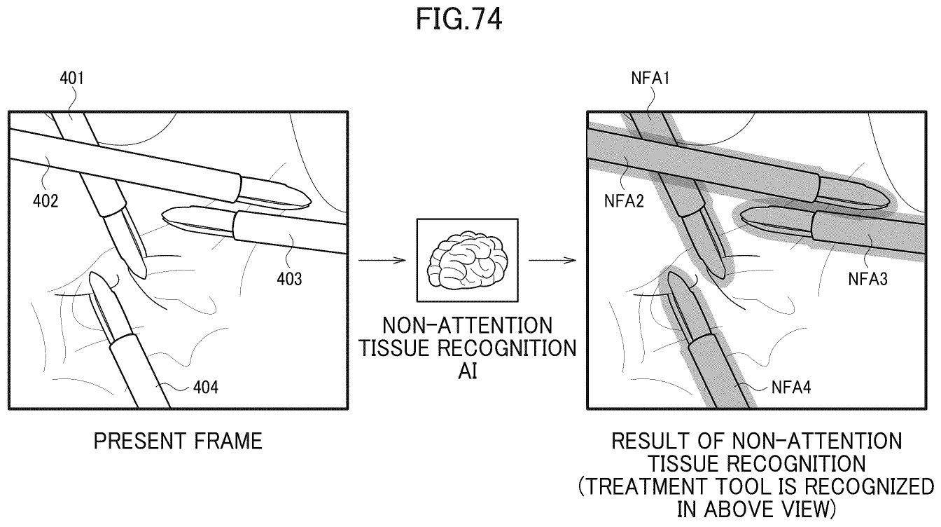

FIG. 74 is a view for describing the non-attention tissue recognizing step S151. The processor 110 recognizes a non-attention tissue region from the input endoscope images. The recognition is segmentation. The non-attention tissue region means a region other than an attention tissue, and is, for example, the treatment tool, a piece of gauze, a tissue that is not the treatment target, or the like. A machine learning model is used for the segmentation. The machine learning model is trained with, for example, endoscope images to which an annotation of an attention tissue region is added as training data. In this case, the processor 110 inputs the endoscope images into the machine learning model to cause the machine learning model to recognize the attention tissue region, and sets the region other than the recognized attention tissue region as the non-attention tissue region. Alternatively, the machine learning model is trained with endoscope images to which an annotation of the non-attention tissue region is added as training data. In this case, the processor 110 inputs the endoscope image into the machine learning model to cause the machine learning model to recognize the non-attention tissue region. FIG. 74 illustrates an example using non-attention tissue recognition AI. Assume that treatment tools 401 to 404 are seen in an endoscope image. The non-attention tissue recognition AI recognizes non-attention tissue regions NFA1 to NFA4 respectively corresponding to the treatment tools 401 to 404.

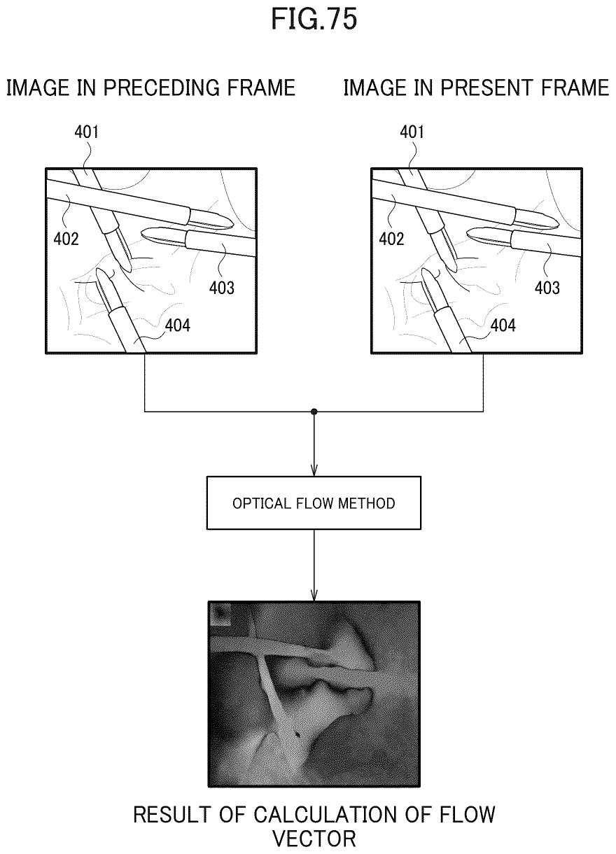

FIG. 75 is a view for describing the flow vector calculating step S152. The processor 110 calculates a flow vector between an endoscope image in a present frame and an endoscope image in a preceding frame by the Optical Flow method. The “preceding frame” may be a frame immediately before the present frame, or a frame before the present frame with a predetermined interval. Alternatively, an interval between the “preceding frame” and the present frame may vary. The Optical Flow method may be a classical non-AI method, or a method using AI such as RAFT. FIG. 75 illustrates, as an example of a result of the calculation of the flow vector, an image in which the magnitude of the flow vector at each point is expressed by brightness and the direction of the flow vector is expressed by a hue.

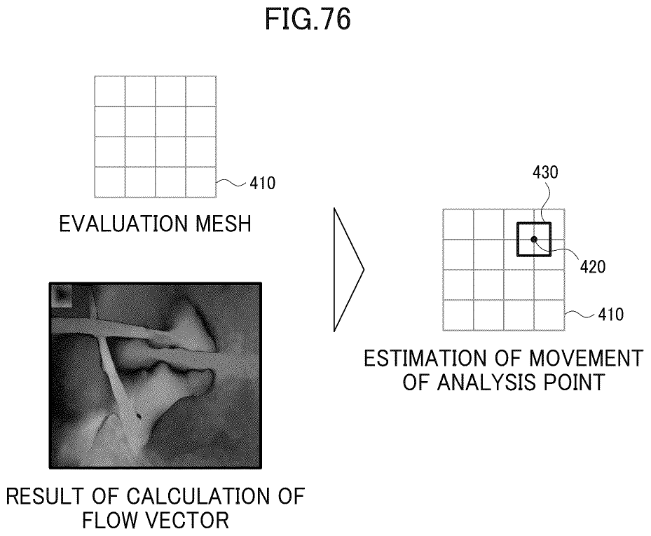

FIG. 76 is a view for describing the evaluation mesh analyzing step S153. The processor 110 estimates the movement of each analysis point of the evaluation mesh 410 based on the flow vector in the endoscope images. The processor 110 may use a flow vector of a pixel on which each analysis point is placed as it is. Alternatively, as illustrated in FIG. 76, the processor 110 may calculate a statistic from the flow vector of a pixel included in a peripheral region 430 of each analysis point 420, and estimate the movement of the analysis point 420 from the statistic. The statistic is, for example, an average value, a median value, a weighted average based on a distance, or the like. With use of the statistics, robustness with respect to a disturbance such as halation, video noise, and mist in analysis of the evaluation mesh increases.

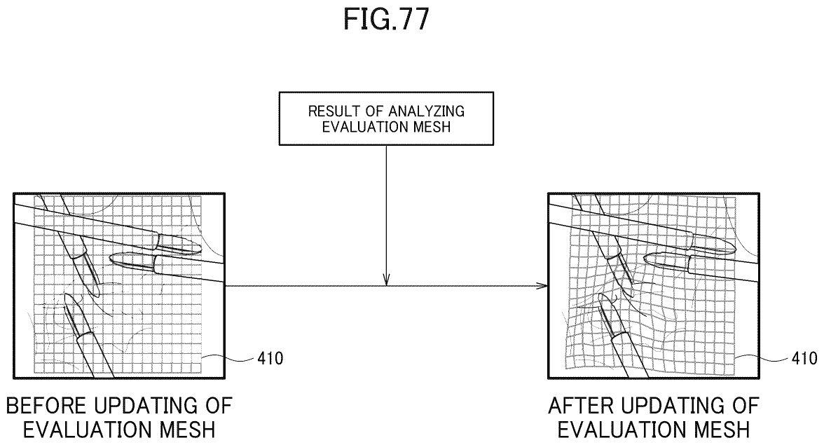

FIG. 77 is a view for describing the evaluation mesh updating step S154. The processor 110 updates the evaluation mesh 410 based on a result of the analysis in the evaluation mesh analyzing step S153. That is, the processor 110 moves each analysis point of the evaluation mesh 410 by the movement quantity of each analysis point, the movement quantity being obtained by the evaluation mesh analyzing step S153.

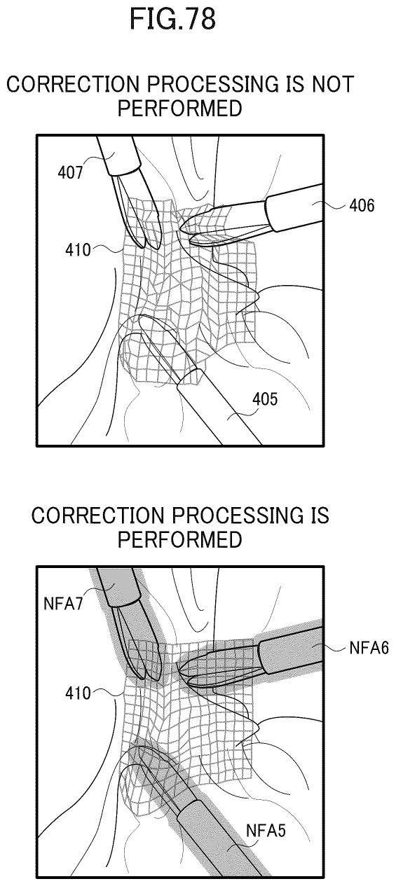

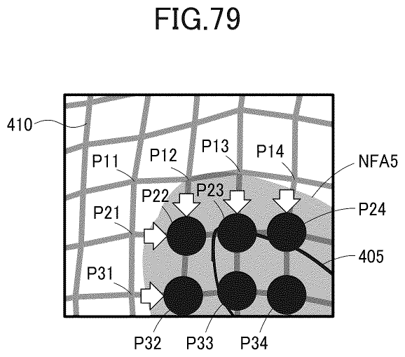

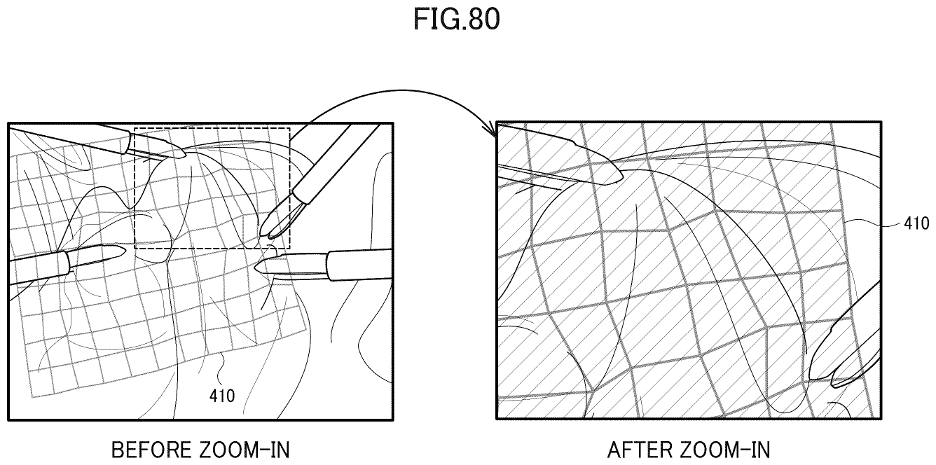

FIGS. 78 and 79 are views for describing modifications of the evaluation mesh analyzing step S153. The flow vector includes not only information regarding the movement of the tissue, but also information regarding the movement of the device. Hence, as illustrated in an upper view of FIG. 78, the movement of the device such as treatment tools 405 to 407 influences the deformation of the evaluation mesh 410. That is, there is a possibility that the evaluation mesh 410 does not represent the deformation of the tissue accurately. To address this, as illustrated in a lower view of FIG. 78, when analyzing the evaluation mesh 410 using the flow vector, the processor 110 performs correction processing to exclude the influence of the movement of the device. In the non-attention tissue recognizing step S151, the processor 110 recognizes non-attention tissue regions NFA5 to NFA7 respectively corresponding to the treatment tools 405 to 407. In the evaluation mesh analyzing step S153, the processor 110 uses the non-attention tissue regions NFA5 to NFA7 to perform the correction processing.

FIG. 79 is a view for describing the correction processing taking the non-attention tissue region NFA5 as an example. A target of the correction processing is analysis points P22 to P24 and P32 to P34 overlapping the non-attention tissue region NFA5. Assume that surrounding analysis points are analysis points P11 to P14, P21, and P31. The processor 110 estimates the movement of an analysis point not overlapping the non-attention tissue region NFA5 using the flow vector. The processor 110 does not estimate the movements of the analysis points P22 to P24 and P32 to P34 overlapping the non-attention tissue region NFA5 using the flow vector. The processor 110 propagates the movement quantities of the surrounding analysis points P11 to P14, P21, and P31 updated with use of the flow vector to estimate the movement quantities of the analysis points P22 to P24 and P32 to P34 overlapping the non-attention tissue region NFA5. A statistic of the movement quantities of a plurality of adjacent analysis points is used in a propagation method. The statistic is, for example, an average value, a median value, a weighted average in consideration of a distance, or the like. Since it is considered that an analysis point with a larger number of surrounding analysis points whose positions have been updated has higher reliability of correction, the processor 110 may perform correction sequentially from such an analysis point. For example, the processor 110 sets the statistics of the movement quantities of the analysis points P11 to P13, P21, and P31 adjacent to the analysis point P22 as an estimation value of the movement quantity of the analysis point P22. Subsequently, the processor 110 sets the statistics of the movement quantities of the analysis points P12 to P14 and P22 adjacent to the analysis point P23 is an estimation value of the movement quantity of the analysis point P23. The estimation value of the movement quantity of P22 calculated earlier is used here.

FIG. 80 is a view for describing a modification of the evaluation mesh updating step S154. As illustrated in FIG. 80, since apparent movement of the object occurs on images when the camera of the endoscope is zoomed in or zoomed out, the apparent movement leads to an increase or decrease of the deformation quantity of the evaluation mesh 410. Hence, even though the object does not deform in reality, information indicating that the deformation quantity increases or decreases is presented. There is a possibility that such an issue occurs due to not only zoom-in or zoom-out, but also due to various kinds of movement of the camera.

To address this, the processor 110 generates and holds an evaluation mesh for calculation of a deformation quantity separately from the normal evaluation mesh, and uses the evaluation mesh for calculation of the deformation quantity to calculate the deformation quantity of the evaluation mesh. The evaluation mesh for calculation of the deformation quantity is an evaluation mesh in which a movement quantity of the camera is corrected to be used for calculation of the deformation quantity of the evaluation mesh. The processor 110 calculates the evaluation mesh for calculation of the deformation quantity in the following three steps.

In a first step, the processor 110 uses an image processing method to estimate a transformation matrix indicating the movement of the camera between the preceding frame and the present frame. In a second step, the processor 110 calculates the transformation matrix indicating the movement of the camera between an initial frame and the present frame. In a third step, the processor 110 uses a transformation matrix indicating the movement of the camera between the initial frame and the present frame to calculate the evaluation mesh for calculation of the deformation quantity.

A specific example of the first step is now described. The transformation matrix indicating the movement of the camera between the preceding frame and the present frame can be estimated by a known technique. For example, a findHomography function in OpenCV or the like can be used. However, since there is an issue that large noise occurs due to the deformation of the tissue in the foreground in an abdominal cavity image, the following method may be combined.

While the tissue on the foreground is deformed by the operator or assistant's operation, it is possible to assume that the other region is approximately stationary on a space. Hence, the processor 110 may separate the foreground and the background from each other in the endoscope images and apply an estimation method using the transformation matrix only to the background. With this configuration, it is possible to estimate the movement of the camera with high accuracy. Examples of a method of separating the foreground and the background from each other include a method using the depth information as illustrated in FIG. 44, a method using segmentation such as GrabCut, and a method using the presence/absence of the movement of the flow vector. Note that in a case of the abdominal cavity image, since light in a space is dominantly light from a light source of the endoscope, it is possible to utilize brightness information based on the inverse square law between illuminance and a distance like the depth information.

A specific example of the second step is now described. As described in the following expression (1), the processor 110 performs accumulation of the transformation matrix indicating the movement of the camera between the preceding frame and the present frame at each time point to calculate the transformation matrix indicating the movement of the camera from the initial frame to the previous frame. In the following expression (1), ^{j}Hᵢ is a transformation matrix indicating the movement of the camera between a frame at a time point i and a frame at a time point j. Assume that a time point in the present frame is t, and a time point in the initial frame is 0.

$$\begin{matrix}[ Expression 1 ] & \\ {}^{t} {H}_{0} = {}^{t} {H}_{t - 1} {}^{t - 1} {H}_{t - 2} … {}^{1} {H}_{0} & ( 1 )\end{matrix}$$

A specific example of the third step is now described. As described in the following expressions (2) and (3), the processor 110 multiplies each point of the evaluation mesh by an inverse matrix of the transformation matrix indicating the movement of the camera from the initial frame to the present frame to calculate the evaluation mesh for calculation of the deformation quantity. Multiplication by the inverse matrix corresponds to projection on a z=1 plane. In the following Expression (2), (^{T}x, ^{T}y) represents a position of the analysis point of the evaluation mesh in a coordinate system of the present frame. In the following Expression (3), (^{o}x, ^{o}y) represents a position of the analysis point of the evaluation mesh for calculation of the deformation quantity in the present frame in a coordinate system of the initial frame. By cancelling the movement of the camera, the position of the analysis point in the present frame in the coordinate system of the initial frame is presented, and the evaluation mesh for calculation of the deformation quantity that reflects only the movement of the object can be obtained.

$$\begin{matrix}[ Expression 2 ] & \\ [ \begin{matrix}{o}_{{x}^{′}} \\ {o}_{{y}^{′}} \\ {o}_{{w}^{′}}\end{matrix} ] = {}^{t} {H}_{0}^{- 1} [ \begin{matrix}{T}_{x} \\ {T}_{y} \\ 1\end{matrix} ] & ( 2 )\end{matrix}$$

$$\begin{matrix}[ Expression 3 ] & \\ [ \begin{matrix}{o}_{x} \\ {o}_{y}\end{matrix} ] = [ \begin{matrix}\frac{{o}_{{x}^{′}}}{{o}_{{w}^{′}}} \\ \frac{{o}_{{y}^{′}}}{{o}_{{w}^{′}}}\end{matrix} ] & ( 3 )\end{matrix}$$

In the present embodiment, the image processing system 100 includes the memory 120. The memory 120 stores a trained model that distinguishes a non-attention region from images. The processor 110 inputs the time-series images to the trained model, and acquires a result of distinguishing the non-attention region from the trained model. The processor 110 calculates the deformation quantity of each cell in the evaluation mesh 410 based on the magnitude and the direction of the movement quantity of each analysis point that does not overlap the non-attention region. Note that the “non-attention region” corresponds to the above-mentioned non-attention tissue region, and is, for example, the treatment tool, a piece of gauze, a tissue that is not the treatment target, or the like in the endoscope images. Note that, for example, the trained model 122 in FIG. 6 includes a region distinguishing model and the non-attention region is estimated by the region distinguishing model.

When the analysis of deformation of the evaluation mesh 410 is influenced by the movement of the non-attention region, there is a possibility that the movement of the treatment target tissue is not tracked accurately. In accordance with the present embodiment, it is possible to perform the analysis of deformation of the evaluation mesh 410 without being influenced by the movement quantity of the analysis point overlapping the non-attention region, whereby it is possible to obtain the evaluation mesh 410 in which the movement of the treatment target tissue is tracked accurately.

Additionally, in the present embodiment, the non-attention region may be a region of the treatment tool in the time-series images.

In accordance with the present embodiment, it is possible to obtain the evaluation mesh 410 in which the movement of the treatment target tissue is tracked accurately without being influenced by the movement of the treatment tool.

In accordance with the present embodiment, the processor 110 may estimate the magnitude and direction of the movement quantity of an analysis point overlapping the non-attention region from the magnitude and direction of the movement quantity of an analysis point not overlapping the non-attention region in the surroundings of the analysis point. Note that, in the example in FIG. 79, P22 to P24 and P32 to P34 correspond to analysis points overlapping the non-attention region.

In accordance with the present embodiment, it is possible to estimate the movement quantity of the analysis point overlapping the non-attention region from the movement quantity of the surroundings, that is, the movement quantity of the tissue in the surroundings. As a result, it is possible to obtain the evaluation mesh 410 that is not influenced by the movement in the non-attention region and in which the movement of the treatment target tissue is estimated accurately.

### 5. Fourth Embodiment

A configuration of the medical system is similar to the configuration of the medical system described with reference to FIG. 5 in the first embodiment. Additionally, a configuration of the image processing system 100 is similar to the configuration of the image processing system 100 described with reference to FIG. 6 in the first embodiment. Methods regarding some functions (1) to (3) will be described below.

### (1) Method of Determining the Start or End of a Function

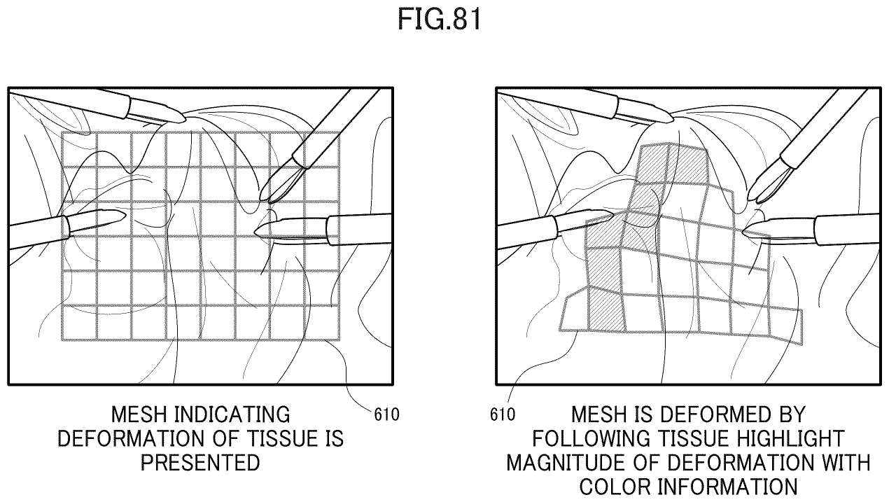

As illustrated in FIG. 81, the processor 110 recognizes the deformation of the tissue associated with the pulling from endoscope images, and presents information regarding the deformation to the operator or the assistant. That is, the processor 110 causes each analysis point of the evaluation mesh 610 to track the deformation of the tissue, and performs display on the monitor so as to superimpose color information or the like corresponding to the deformation of cells of the evaluation mesh 410 on the endoscope images.

To present the deformation of the tissue along with the pulling, it is necessary to set a start point and end point of a presentation function, that is, a criterion state of deformation and an end point of display in a series of treatment. It is considered that a surgeon, who is the operator or the assistant, performs external input, but both hands of the surgeon are basically busy in operating the device during the treatment, and has difficulty to perform external input.

To address this, the processor 110 recognizes an action state of the operator or assistant's forceps or treatment tool from the endoscope images, and uses a result of the recognition to set a start timing or end timing of the function. With this configuration, it is possible to narrow down to a necessary timing for the surgeon, and present the pulling information without a special additional operation. Since the medical system automatically sets the start timing or end timing of the function without intervention of the surgeon, it is possible to reduce an additional operation by the surgeon. Two examples (1a) and (1b) are described below.

### (1a) Preliminary Registration of a Pattern

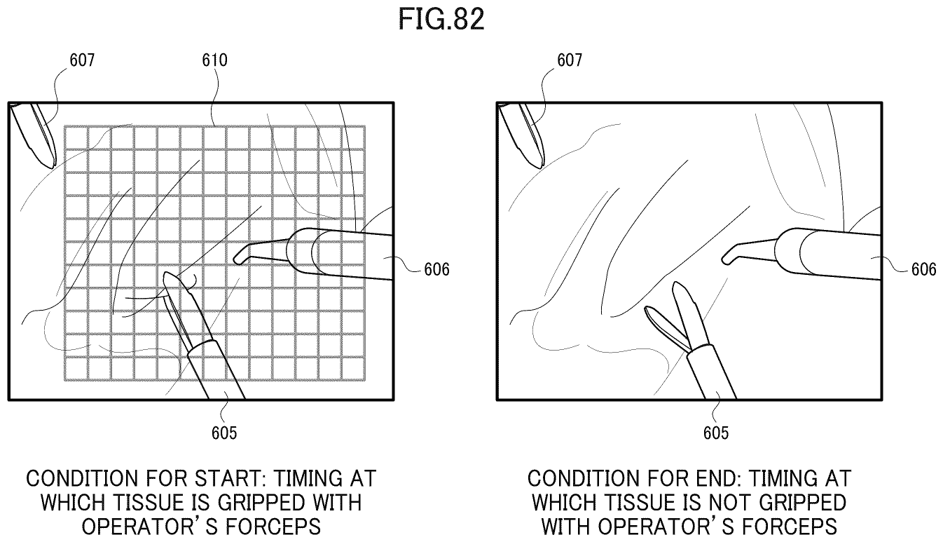

Patterns (conditions) corresponding to manipulations and scenes are preliminarily registered in the image processing system 100. For example, the memory 120 in FIG. 6 stores the preliminarily registered patterns (conditions). The processor 110 starts the function in a case where a condition of a pattern is satisfied, and ends the function in a case where the condition of the pattern is not satisfied. FIG. 82 illustrates an example of the pattern. In FIG. 82, the assistant's forceps 607, the operator's forceps 605, and an energy treatment tool 606 are seen, and a condition that the operator grips the tissue with the forceps 605 serves as a pattern for an energy treatment. That is, when recognizing that the forceps 605 grip the tissue, the processor 110 starts the function and sets the evaluation mesh 610. When recognizing that the forceps 605 do not grip the tissue, the processor 110 ends the function. The pattern is not limited thereto, and may be various patterns depending on manipulations or scenes. For example, a condition that the assistant grips the tissue with forceps held by both hands may serve as a pattern for development of the surgical field. The preliminarily registered pattern may be updated afterwards. Examples of conceivable methods of recognizing the pattern from an image or the like include a method of recognizing a state of gripping the tissue with the forceps or the treatment tool.

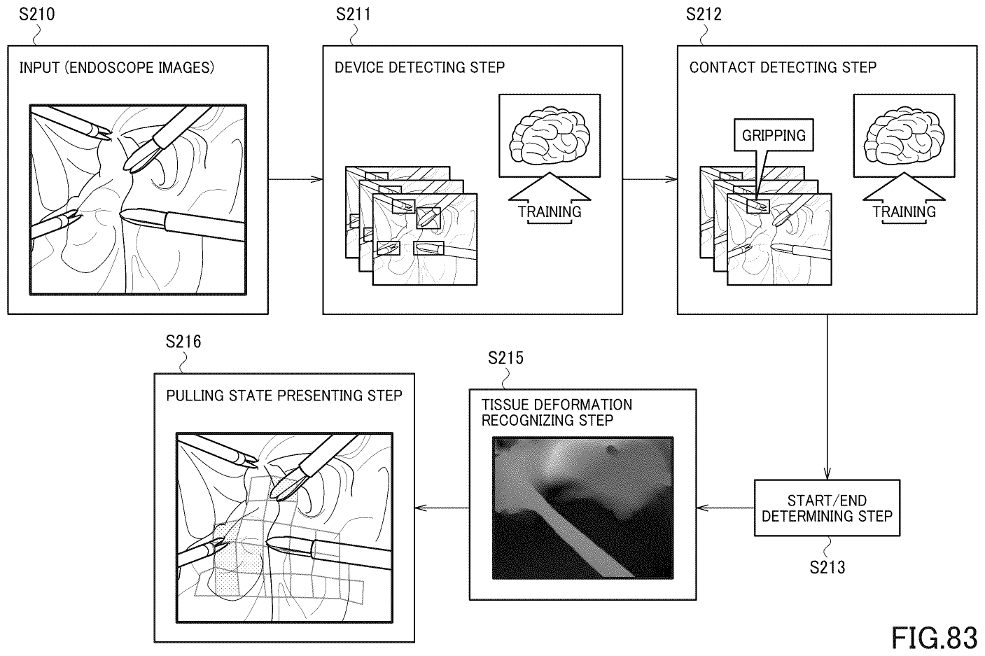

FIG. 83 illustrates a flow of processing in the image processing system 100 in the fourth embodiment. In steps S210 to S213, the processor 110 detects that the forceps or the treatment tool comes in contact with (grips) the tissue from the endoscope images, and starts or ends the function of a tissue deformation recognizing step S215 based on a preliminarily determined rule. The processor 110 recognizes information regarding deformation of the tissue in the tissue deformation recognizing step S215, and presents the information to the operator or the assistant in a pulling state presenting step S216. Details of each step will be described below.

Note that steps S210 to S213 correspond to the pulling scene determining step S21 in FIG. 7, step S215 corresponds to the pulling range estimating step S23 in FIG. 7, and step S216 corresponds to the presentation information processing step S25 in FIG. 7. Processing described in steps S215 and S216 is merely an example, and the methods in the pulling range estimating step S23 and the presentation information processing step S25 described in the first embodiment and the like may be adopted as appropriate.

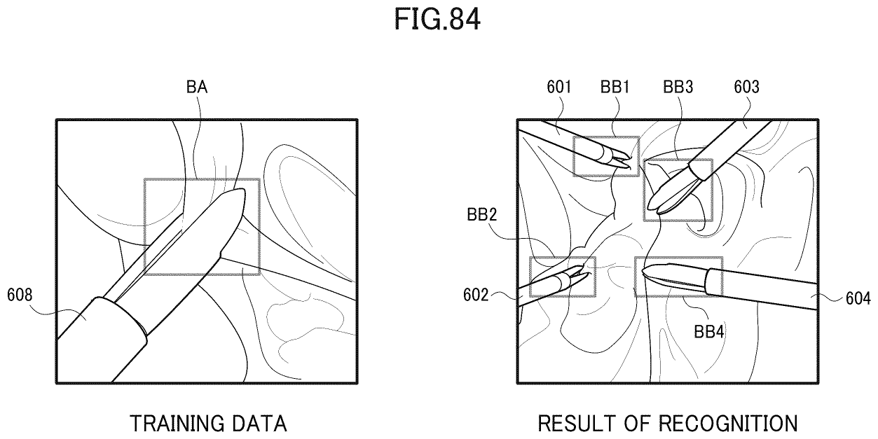

In step S210, the endoscope images are input to the processor 110. In a device detecting step S211, the processor 110 detects, from the input endoscope image, the forceps, or the treatment tool in the images. A network is utilized for detection. The network has been subjected to machine learning with training data in which an annotation of the position of the forceps or the position of the treatment tool is added to endoscope images. FIG. 84 illustrates an example regarding the training data and a recognition result taking a case of using an object detection model for example. In the example of the training data, forceps 608 are seen in the endoscope images, and an annotation of a boundary box BA is added to a jaw of the forceps 608. In a training phase, training of the network is performed using multitudes of endoscope images on which annotation has been performed. In an inference phase, the processor 110 inputs the endoscope images to the network. In this example, forceps 601 to 604 are seen in an endoscope image. The network detects jaws of the forceps 601 to 604, and outputs boundary boxes BB1 to BB4 indicating the positions of the jaws as attention regions. Note that segmentation may be used in detection of the forceps or the treatment tool.

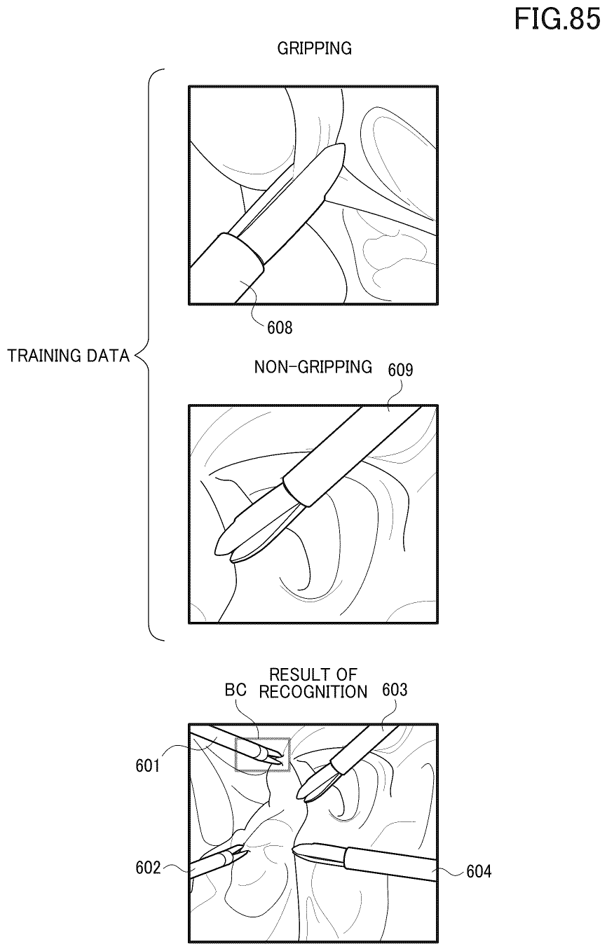

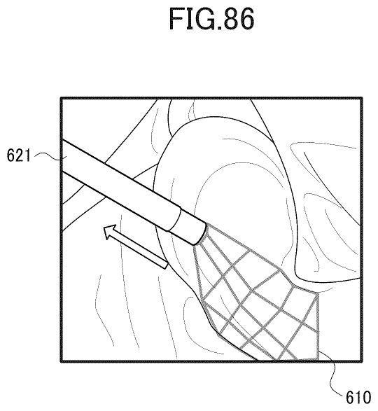

In a contact detecting step S212, the processor 110 detects a contact state of the tissue based on the attention region extracted in the device detecting step S211. A network is utilized for detection. The network has been subjected to machine learning with training data in which a contact state between the forceps or the treatment tool and the tissue is labeled in endoscope images. The contact state may be, for example, contact/non-contact between the jaw and the tissue, or gripping/non-gripping of the tissue with the jaw. In the latter case, in a case where the jaw grips the tissue, it is detected as contact. FIG. 85 illustrates an example regarding the training data and a recognition result. The training data includes an endoscope image that shows a state where the forceps 608 grip the tissue and an endoscope image that shows a state where forceps 609 do not grip the tissue. The former endoscope image is labeled as “GRIPPING”, and the latter endoscope image is labeled as “NON-GRIPPING”. In a training phase, training of the network is performed using multitudes of labeled endoscope images. In an inference phase, the processor 110 inputs the endoscope image to the network. In this example, the forceps 601 to 604 are seen in an endoscope image. The network detects gripping states of the forceps 601 to 604, and outputs a boundary box BC indicating the position of the jaw of the forceps 601 determined as gripping.

In a start/end determining step S213, the processor 110 determines the start/end of the recognition in the tissue deformation recognizing step S215 based on the contact state between the forceps or the treatment tool and the tissue, the contact state being detected in the contact detection step S212, and a preliminarily set condition. The preliminarily set condition is as described with reference to FIG. 82.

In the tissue deformation recognizing step S215, the processor 110 recognizes the deformation of the tissue associated with the pulling with the forceps or the like from the endoscope images. As illustrated in FIG. 86, the processor 110 sets an evaluation mesh 610 in the endoscope images. The processor 110 performs tracking of a characteristic point with respect to each analysis point of the evaluation mesh 610, and thereby recognizes the behavior of the tissue associated with the pulling with forceps 621 from the endoscope images. The processor 110 acquires a deformation quantity of each analysis point or a deformation quantity of each cell from the evaluation mesh 610.

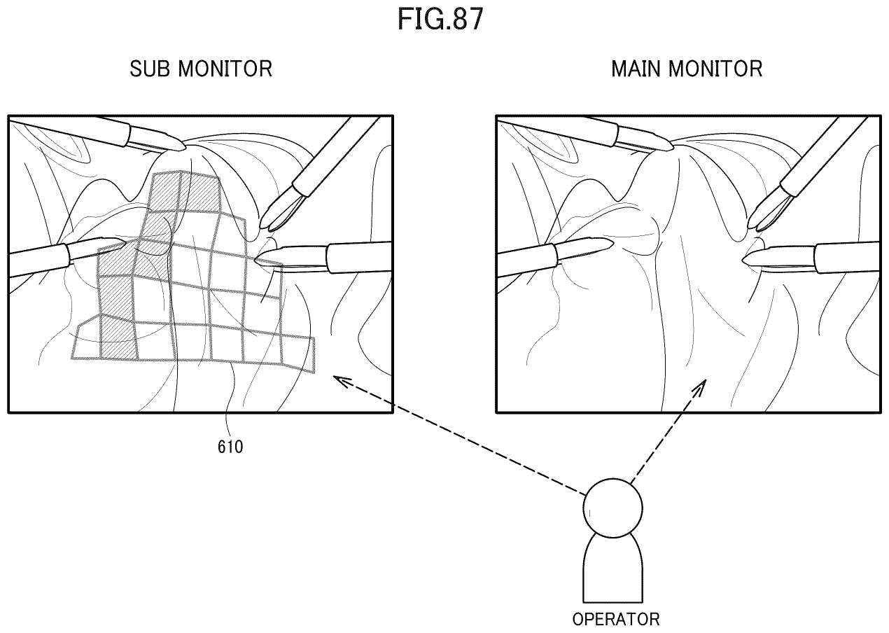

In the pulling state presenting step S216, the processor 110 highlights each cell of the evaluation mesh 610 with a color or the like based on the deformation quantity of the evaluation mesh 610, which is obtained in the tissue deformation recognizing step S215. This highlight display is superimposed on the endoscope images and displayed on a monitor. With this display, information regarding the deformation of the tissue is presented to the operator or the assistant. As illustrated in FIG. 87, the medical system may include a main monitor and a sub monitor. The processor 110 may display endoscope images on which the information regarding the deformation of the tissue is not superimposed on the main monitor, and display endoscope images on which the information regarding the deformation of the tissue is superimposed on the sub monitor. FIG. 87 illustrates an example in which, out of the operator as the surgeon and the assistant, the surgeon sees the monitors, but the assistant may see the monitors. Alternatively, the medical system may include a monitor for the operator and a monitor for the assistant. The processor 110 may display information appropriate for each monitor. For example, the processor 110 may display endoscope images on which information regarding the deformation of the tissue appropriate for the operator is superimposed on the monitor for the operator, and display endoscope images on which information regarding the deformation of the tissue appropriate for the assistant is superimposed on the monitor for the assistant.

In accordance with the above-mentioned method, it is possible to narrow down to a necessary timing for the surgeon to present the pulling information. Additionally, since the image processing system 100 automatically sets the timing, it is possible to reduce an additional operation by the surgeon.

### (1b) Storage of a History

Basically similarly to the method (1a), the processor 110 may recognize the state of gripping/non-gripping the tissue with the forceps in the contact detecting step S212, and store a recognition result in the memory 120 in FIG. 6 or the like until the recognition result is switched. For example, in a case of recognizing the gripping, the processor 110 stores a result of recognizing the gripping in the memory 120, does not update the memory 120 until recognizing the non-gripping, and updates the memory 120 with a result of recognizing the non-gripping when recognizing the non-gripping.

There is a conceivable case where the forceps disappear to the outside of the field of view of the camera due to the movement of the camera or the pulling of the tissue with the forceps. Such a state is assumed to occur especially in the case of the assistant's forceps. In the method of recognizing the gripping or the non-gripping from the endoscope images, in a case where the forceps move to the outside of the field of view of the camera, it is impossible to recognize the gripping or the non-gripping accurately.

To address this, regarding the gripping/non-gripping state, the processor 110 stores a temporary recognition result in the memory 120 and uses the temporary recognition result until the recognition result based on the endoscope images is updated, and can thereby apply the function even in a case where the forceps move to the outside of the field of view. Even the forceps that have been outside the field of view once are surely within the field of view when gripping the tissue, so that the gripping can be detected from the endoscope images. Meanwhile, it is considered that the forceps change from the gripping state to the non-gripping state outside the field of view. However, such a case poses no problem because the surgeon himself/herself notices the change. In a case where the forceps in the non-gripping state move to the field of view the next time, the function ends.

### (2) Assistance for the Assistant by Presenting Loosening of the Assistant's Pulling

There is a case where the assistant is unable to notice the loosening of the tissue associated with the operator's treatment and is unable to maintain appropriate pulling. The assistant does not know how much he/she should pull the tissue to return the loosened tissue to the appropriate pulling state. This results in increase of instructions from the operator to the assistant.

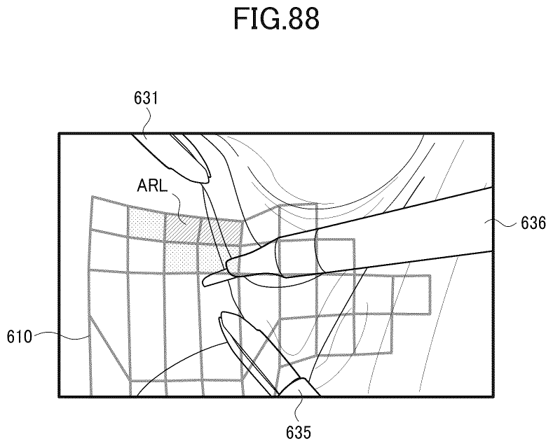

To address this, the processor 110 presents the loosening of the tissue associated with the operator's treatment with color information or the like on the monitor. FIG. 88 illustrates an example. The assistant's forceps 631, the operator's forceps 635, and the energy treatment tool 636 are seen in an endoscope image. The processor 110 adds a color to cells in which deformation corresponding to the loosening of the tissue occurs based on the analysis of the deformation of the evaluation mesh 610. In FIG. 88, the cells to which the color is added are indicated as a hatched region ARL. The assistant pulls the tissue with the forceps 631 so that the color of the region ARL disappears, that is, the loosening of the tissue is resolved. In this manner, in accordance with the present embodiment, the pulling is adjusted so as to make the assistant aware of the loosening of the tissue and eliminate the presented color information or the like, thereby allowing the assistant to autonomically return to the appropriate pulling state.

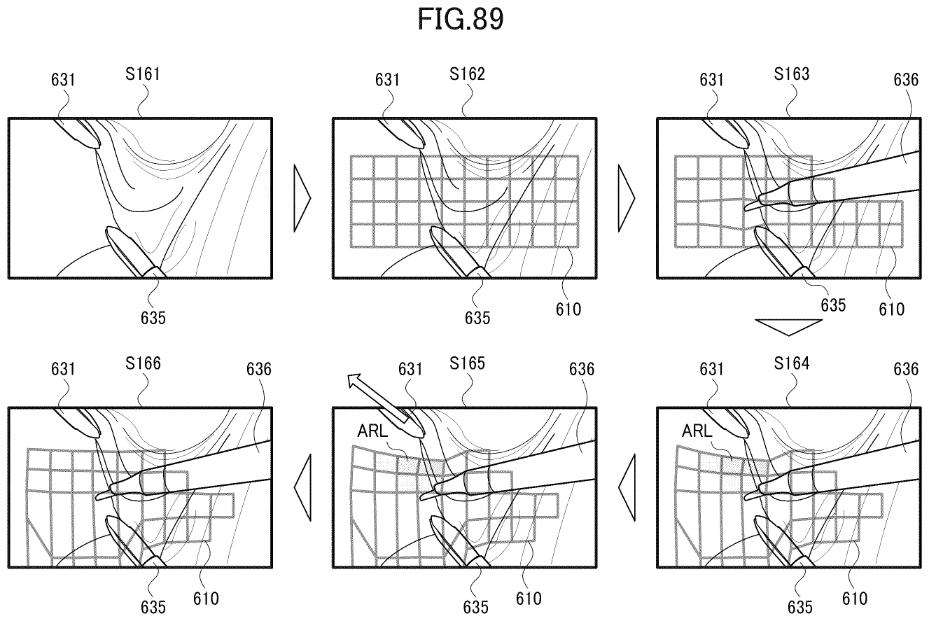

FIG. 89 illustrates a first flow of presentation of loosening. In step S161, the function of presenting the pulling state is OFF. The assistant grips and pulls the tissue with the forceps 631. In step S162, when detecting that the assistant has completed the development of the tissue, the processor 110 presents the evaluation mesh 610 indicating the deformation of the tissue. The evaluation mesh 610 herein is in a criterion state for a regular shape. In step S163, the processor 110 causes each analysis point of the evaluation mesh 610 to follow the deformation of the tissue to deform the evaluation mesh 610. The processor 110 may narrow down the evaluation region as described in the second embodiment. FIG. 89 illustrates an example in which the narrowing has been performed. In step S164, the processor 110 determines the region ARL in which the tissue has been loosened based on the deformation quantity of each cell in the evaluation mesh 610, and presents the region ARL by adding color information to cells in the region ARL. In step S165, the assistant pulls the tissue with the forceps 631 so as to eliminate the color information regarding the region ARL. As illustrated in step S165, in a case where the cells of the evaluation mesh 610 in the region ARL returns to or becomes closer to the criterion state, the processor 110 cancels the presentation of the color information.

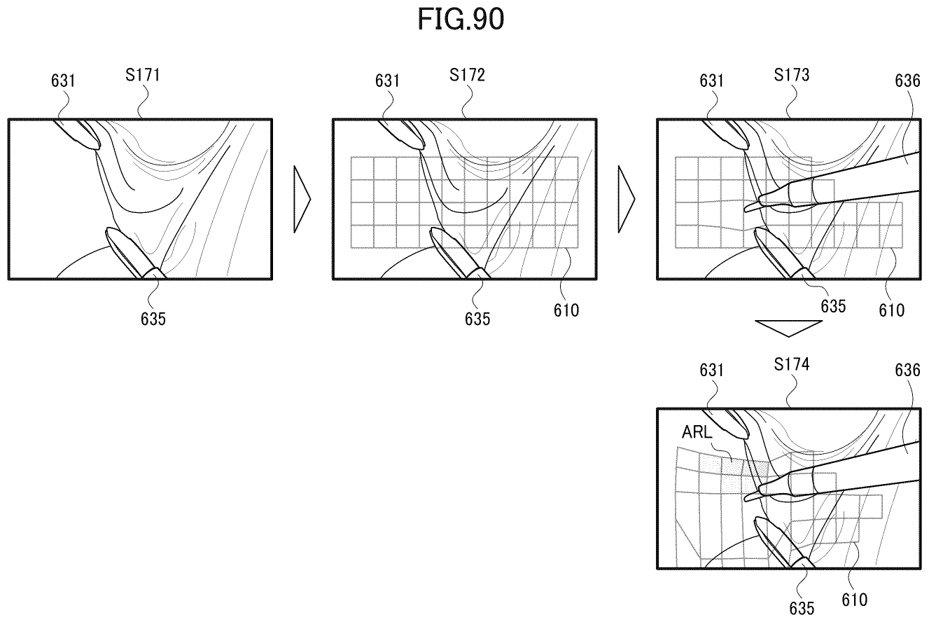

FIG. 90 illustrates a second flow of presentation of loosening. In step S171, the function of presenting the pulling state is OFF. The assistant grips and pulls the tissue with the forceps 631. In step S172, when detecting that the assistant has completed the development of the tissue, the processor 110 disposes the evaluation mesh 610 for tracking the deformation of the tissue. The evaluation mesh 610 may be or may not be displayed, and is used for calculation for tracking of the deformation of the tissue. The evaluation mesh 610 herein is in a criterion state for a regular shape. In step S173, the processor 110 causes each analysis point of the evaluation mesh 610 to follow the deformation of the tissue to deform the evaluation mesh 610. The processor 110 calculates the deformation quantity of each cell. The processor 110 may narrow down the evaluation region as described in the second embodiment. FIG. 90 illustrates an example in which the narrowing has been performed. In step S174, the processor 110 determines the region ARL in which the tissue has been loosened based on the deformation quantity of each cell in the evaluation mesh 610, and presents the region ARL on the monitor by adding color information to cells in the region ARL. The processor 110 may present only the color information regarding the region ARL without presenting the evaluation mesh 610, or may present the color information regarding the region ARL together with the evaluation mesh 610. In a case where the evaluation mesh 610 is not presented in step S172, the information is presented for the first time when the loosening is determined in step S174.

### (3) Assistance for the Operator's Pulling by Presenting the Deformation of the Tissue

A non-expert operator does not know that the pulling with the forceps held by the left hand is insufficient. For example, the non-expert operator tends to pay little attention to the forceps held by the left hand, and is thereby unable to notice the pulling being insufficient. The non-expert operator does not know at least how much he/she should pull the tissue depending on a scene or a tissue. As a result, the pulling with the forceps held by the left hand weakens at the time of use of a monopolar treatment tool or the like, and heat diffuses to a surrounding tissue of a tissue to be treated by the monopolar treatment tool.

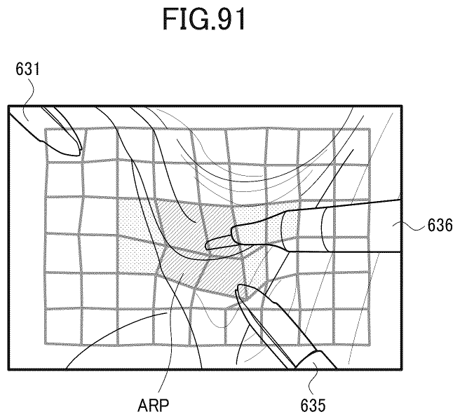

To address this, the processor 110 presents a range of the tissue deformed as a result of the pulling with the forceps held by the left hand of the operator using color information. FIG. 91 illustrates an example. The assistant's forceps 631, the operator's forceps 635, and an energy treatment tool 636 are seen in an endoscope image. The processor 110 adds a color to cells indicating the deformation of the tissue due to the pulling with the operator's forceps 635 based on the analysis of the deformation of the evaluation mesh 610. In FIG. 91, the cells to which the color is added are indicated as a hatched region ARP. The operator easily notices whether the pulling by the forceps 635 held by the left hand is appropriate by seeing the color information regarding the region ARP. In accordance with the present embodiment, the frequency of execution of the treatment on the tissue in a state where pulling of the tissue is weak decreases, and it is possible to inhibit heat diffusion due to insufficient pulling.

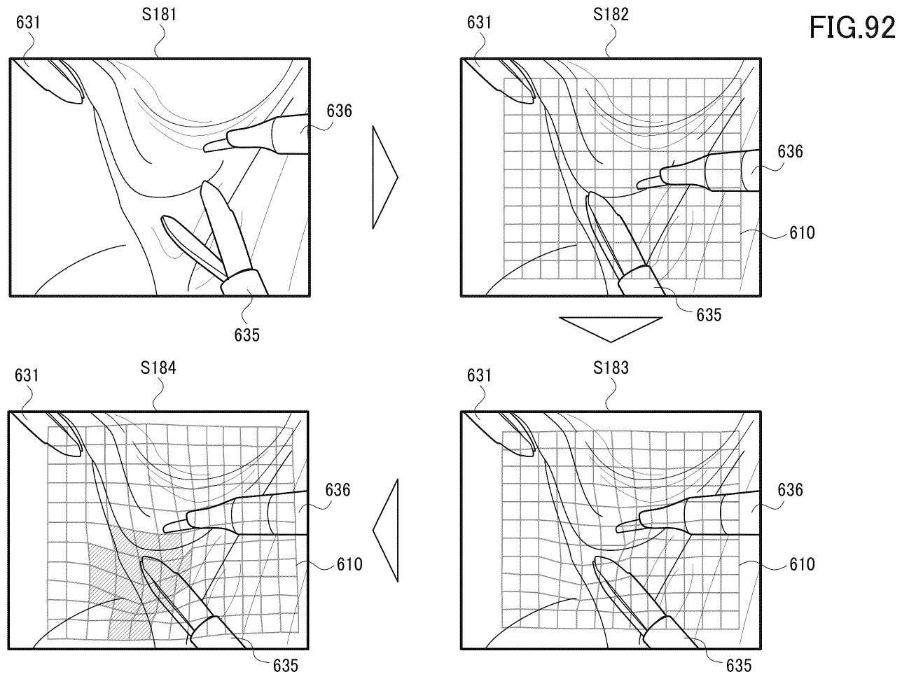

FIG. 92 illustrates a first flow of assistance for the operator's pulling. In step S181, the function of presenting the pulling state is OFF. This is a state before the operator grips the tissue with the forceps 635. In step S182, when detecting that the operator has completed the gripping of the tissue with the forceps 635, the processor 110 presents the evaluation mesh 610 indicating the deformation of the tissue. The evaluation mesh 610 herein is in a criterion state for a regular shape. In step S183, the processor 110 causes each analysis point of the evaluation mesh 610 to follow the deformation of the tissue to deform the evaluation mesh 610. The processor 110 presents a region with a higher deformation quantity of cells in a darker color. In the present example, a state where the deformation quantity of the cells is low in step S183 and almost no color is added is taken for example. As illustrated in step S184, as the operator further pulls the tissue with the forceps 635 and the deformation of the tissue becomes larger, a region to which the color is presented extends.

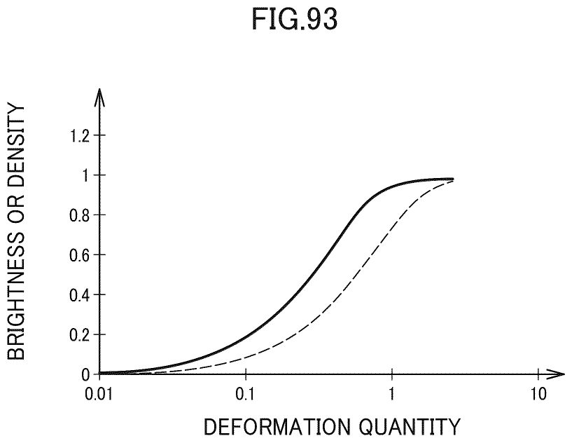

A second flow of assistance for the operator's pulling is now described. As a flowchart, FIG. 92 similar to the first flow is used. In step S181, the function of presenting the pulling state is OFF. In step S182, when detecting that the operator has completed the gripping of the issue with the forceps 635, the processor 110 disposes the evaluation mesh 610 indicating the deformation of the tissue. The evaluation mesh 610 may be or may not be displayed, and is used for calculation for tracking of the deformation of the tissue. In step S183, the processor 110 causes each analysis point of the evaluation mesh 610 to follow the deformation of the tissue to deform the evaluation mesh 610. The processor 110 calculates the deformation quantity of each cell, and presents a region with a higher deformation quantity of cells in a darker color. FIG. 93 illustrates an example of a relationship between the deformation quantity and the brightness or density of a color. In the present example, a sigmoid function is used. When the deformation quantity is x, the sigmoid function is expressed by f(x)=1/(1+e^{-ax}), where a is a freely selected positive coefficient. FIG. 93 illustrates two sigmoid functions with different values of a with a solid line and a dotted line. In step S183 in FIG. 92, a state where the deformation quantity of the cells is low and almost no color is added is taken for example. As illustrated in step S184, as the operator further pulls the tissue with the forceps 635 and the deformation of the tissue becomes larger, a region in which the color is presented extends and the presented color becomes darker.

Although the embodiments to which the present disclosure is applied and the modifications thereof have been described in detail above, the present disclosure is not limited to the embodiments and the modifications thereof, and various modifications in components may be made in an implementation phase without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

1. An image processing system comprising:
one or more processors comprising hardware configured to:
sequentially acquire time-series images captured by an endoscope;
dispose an evaluation mesh including a plurality of analysis points in a freely-selected timing image out of the time-series images;
deform the evaluation mesh in each image of the time-series images so that each analysis point in each image of the time-series images tracks a characteristic point of an object located on each analysis point in the freely-selected timing image in which the evaluation mesh is disposed;
calculate a deformation quantity of each cell of the evaluation mesh based on magnitude and a direction of a movement quantity of each analysis point in each image; and
present information regarding deformation of the evaluation mesh based on the calculated deformation quantity.

2. The image processing system as defined in claim 1, wherein the one or more processors superimpose a display in a mode depending on the deformation quantity in each image of the time-series images on each image.

3. The image processing system as defined in claim 2, wherein the one or more processors superimpose the display in which each cell is colored depending on the deformation quantity of each cell, on each image.

4. The image processing system as defined in claim 1, wherein
the one or more processors being configured to:
control a first monitor to display the time-series images, and
control a second monitor to display the time-series images and the information regarding deformation of the evaluation mesh.

5. The image processing system as defined in claim 1, wherein the one or more processors being configured to determine an evaluation region, which is a region on which the evaluation mesh is disposed or a region to be reflected on an image display out of the evaluation mesh, based on at least one of a movement quantity of the object between the time-series images, an image characteristic quantity of the object, or depth information of the object.

6. The image processing system as defined in claim 5, wherein
the one or more processors being configured to:
calculate, at each point of the object in the time-series images, a cumulative movement quantity obtained by accumulation of a quantity of movement between frames or at predetermined intervals since a criterion timing until elapse of a predetermined period of time, and
exclude a region in which the cumulative movement quantity is a threshold or less to set the evaluation region.

7. The image processing system as defined in claim 5, wherein
the one or more processors being configured to:
calculate a quantity of movement of each point of the object in the time-series images,
aggregate the movement quantity of each point in images to calculate a total value,
accumulates the total value since a criterion timing to calculate a total movement quantity,
when the total movement quantity exceeds a first threshold, calculates a cumulative movement quantity obtained by accumulation of the movement quantity of each point between frames or at predetermined intervals since the criterion timing until the total movement quantity exceeds the first threshold, and
exclude a region in which the cumulative movement quantity is a second threshold or less to set the evaluation region.

8. The image processing system as defined in claim 5, wherein
the one or more processors being configured to:
input the time-series images to a trained model that performs segmentation to estimate the evaluation region from the endoscope images, and
set the evaluation region from a result of estimation from the trained model.

9. The image processing system as defined in claim 5, wherein
the one or more processors being configured to:
input the time-series images to a trained model that performs segmentation to divide an image into regions, and
set the evaluation region in, out of a plurality of regions divided by the trained model, a region with maximum overlap with a predetermined region in the image.

10. The image processing system as defined in claim 5, wherein
the one or more processors being configured to:
perform edge detection processing on the time-series images, and
set a closed region closed with a detected edge as the evaluation region.

11. The image processing system as defined in claim 5, wherein
the one or more processors being configured to:
detect a pulling direction of a treatment tool that pulls the object from the time-series images, and
set the evaluation region on an opposite side of the pulling direction with respect to a predetermined position on the treatment tool.

12. The image processing system as defined in claim 5, wherein
the one or more processors being configured to:
acquire distribution of depths from the endoscope to the object, and
set the evaluation region in a region on a smaller depth side with a line on which a depth significantly changes in the distribution of depths as a boundary.

13. The image processing system as defined in claim 1,
further comprising a memory that stores a trained model that distinguishes a non-attention region from an image,
wherein the one or more processors being configured to:
input the time-series images to the trained model,
acquire a result of distinguishing the non-attention region from the trained model, and
calculate the deformation quantity of each cell of the evaluation mesh based on the magnitude and the direction of the movement quantity of an analysis point not overlapping the non-attention region.

14. The image processing system as defined in claim 13, wherein the non-attention region is a region of a treatment tool in the time-series images.

15. The image processing system as defined in claim 13, wherein the one or more processors being configured to estimate the magnitude and the direction of the movement quantity of an analysis point overlapping the non-attention region from the magnitude and the direction of the movement quantity of the analysis point not overlapping the non-attention region in a surrounding of the analysis point.

16. The image processing system as defined in claim 1, wherein
the one or more processors being configured to: determine whether or not a scene is a pulling scene in which the object is pulled with a treatment tool based on the time-series images or a user's input, and
when determining that the scene is the pulling scene, perform the deformation analysis processing on the evaluation mesh to analyze deformation of the object due to pulling.

17. The image processing system as defined in claim 1, wherein
the one or more processors configured to:
determine a region of the object in which tension is applied by the pulling based on the deformation quantity of each cell in each image of the time-series images, and
superimpose a display indicating the determined region on each image.

18. The image processing system as defined in claim 5, wherein the one or more processors being configured to determine to exclude part of the evaluation region or maintain the evaluation region based on at least one of the movement quantity, the image characteristic quantity, or the depth information at every given update timing to update the evaluation region.

19. An image processing method comprising:
sequentially acquiring time-series images captured by an endoscope;
disposing an evaluation mesh including a plurality of analysis points in a freely-selected timing image out of the time-series images;
deforming the evaluation mesh in each image of the time-series images so that each analysis point in each image of the time-series images tracks a characteristic point of an object located on each analysis point in the freely-selected timing image in which the evaluation mesh is disposed;
calculating a deformation quantity of each cell of the evaluation mesh based on magnitude and a direction of a movement quantity of each analysis point in each image; and
presenting information regarding deformation of the evaluation mesh based on the calculated deformation quantity.

20. A non-transitory information storage medium storing a program that causes a computer to execute:
sequentially acquiring time-series images captured by an endoscope;
disposing an evaluation mesh including a plurality of analysis points in a freely-selected timing image out of the time-series images;
deforming the evaluation mesh in each image of the time-series images so that each analysis point in each image of the time-series images tracks a characteristic point of an object located on each analysis point in the freely-selected timing image in which the evaluation mesh is disposed;
calculating a deformation quantity of each cell of the evaluation mesh based on magnitude and a direction of a movement quantity of each analysis point in each image; and
presenting information regarding deformation of the evaluation mesh based on the calculated deformation quantity.
